(19) United States
(12) Patent Application Publication
(10) Pub. No.: US 2011/0112169 A1
(43) Pub. Date: May 12, 2011

(54) RNAi-BASED THERAPEUTICS FOR ALLERGIC RHINITIS AND ASTHMA

(75) Inventors: Jianzhu Chen, Lexington, MA (US); Herman N. Eisen, Waban, MA (US); Qing Ge, Cambridge, MA (US)

(73) Assignee: Massachusetts Institute of Technology, Cambridge, MA (US)

(21) Appl. No.: 12/891,626

(22) Filed: Sep. 27, 2010

Related U.S. Application Data
(63) Continuation of application No. 11/069,611, filed on Mar. 1, 2005, now abandoned.
(60) Provisional application No. 60/549,070, filed on Mar. 1, 2004.

(51) Int. Cl. A61K 31/713; C07H 21/02; A61K 31/7105; A61P 11/00; A61P 11/06; C12N 15/63

(52) U.S. Cl. 514/44 A; 536/24.5; 435/320.1

(57) ABSTRACT

The present invention provides compositions comprising one or more RNAi agents (e.g., siRNAs, shRNAs, or RNAi vectors) for the treatment of conditions and diseases mediated by (e.g., featuring IgE-mediated hypersensitivity), as well as systems for identifying RNAi agents effective for this purpose. The compositions are suitable for the treatment of allergic rhinitis and/or asthma. In certain embodiments of the invention the RNAi agent is targeted to a transcript that encodes a protein selected from the group consisting of the FCεRIα chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2. In addition, the invention provides RNAi agent/delivery agent compositions and methods of use. In certain embodiments of the invention compositions comprising an RNAi agent are delivered by the respiratory route.

### CROSS-REFERENCE TO RELATED APPLICATION

This application is a continuation of and claims the benefit of priority under 35 U.S.C. §120 to U.S. patent application Ser. No. 11/069,611, filed Mar. 1, 2005, and published on Mar. 16, 2006, as U.S. Patent Publication Number 2006/0058255 (“the '611 application”).

The '611 application is a non-provisional of and claims the benefit of priority under 35 U.S.C. §119(e) to U.S. Provisional Patent Application 60/549,070, filed Mar. 1, 2004 (“the '070 application”). The entire contents of the '611 application and the '070 application are hereby incorporated herein by reference.

### GOVERNMENT SUPPORT

The United States Government has provided grant support utilized in the development of the present invention. In particular, National Institutes of Health grant numbers 5-RO1-A144477, 5-RO1-A144478, 5—ROI-CA60686, 1-RO1-A150631, and RO1-AI40146 have supported development of this invention. The United States Government may have certain rights in the invention.

### BACKGROUND OF THE INVENTION

Allergic diseases, such as allergic rhinitis and asthma, are widely believed to be caused at least in part by hypersensitivity reactions to otherwise generally innocuous foreign substances (allergens). Allergic rhinitis and asthma differ in the primary locations where the allergic reactions take place: the nasal mucosa for allergic rhinitis and the lower respiratory tract for asthma. Approximately 10% of the U.S. population is afflicted by allergic rhinitis. More patients visit doctors for allergic diseases than for any other medical problem.

Allergies are also the largest cause of time lost from work and school, and their impact on personal lives and direct and indirect costs to the medical system and economy are enormous. It is estimated that in industrialized countries one in five to ten individuals is affected by asthma, and the incidence has risen dramatically over the past two decades (Umetsu, D., et al., Nature Immunology, 3(8): 715-720, 2002).

Both allergic rhinitis and asthma involve the release of pharmacologically active mediators from mast cells and basophils. The mediators cause smooth-muscle contraction and the increased vascular permeability and vasodilation that result in typical allergic symptoms such as runny nose and watery eyes. These mediators are also directly or indirectly involved in development of asthmatic symptoms such as airway hyperresponsiveness, mucus secretion, and airway obstruction. Asthma is typically characterized by both acute and chronic inflammation, and it has been suggested that acute inflammation is primarily responsible for episodic bronchoconstriction while chronic inflammation and airway wall remodeling contribute to airway hyperreactivity and fixed airflow obstruction that frequently occurs in chronic asthmatics (Bousquet, J., et al., Am. J. Crit. Care Med., 161:1720-1745, 2000).

Many medications, such as antihistamines, corticosteroids, and decongestants are available for temporary relief of allergic rhinitis symptoms. Allergen immunotherapy can be curative, particularly in children, but is not effective for about half of treated patients and typically requires a large number of injections for successful completion. Asthma can also be treated using a variety of agents including bronchodilators and corticosteroids. However, none of these therapies is fully effective, and many of them have unwanted side effects. Therefore there remains a need in the art for effective therapy and prophylaxis for allergic rhinitis and asthma.

### SUMMARY OF THE INVENTION

The present invention provides novel therapeutic agents for the treatment of a variety of diseases and conditions in which IgE, and/or cells that produce IgE, plays a major role. In particular, the invention provides novel therapeutics for diseases or conditions associated with IgE-mediated hypersensitivity (also referred to as Type I hypersensitivity), e.g., allergic rhinitis and asthma and the amelioration of their manifestations (e.g., symptomatic relief). The therapeutic agents are based on RNAi, a phenomenon in which double-stranded RNA containing a portion that is complementary to a target RNA leads to inhibition of the target RNA when present in a cell. The mechanism of RNAi generally involves cleavage of the target RNA or inhibition of its translation. The RNAi agents of the invention inhibit expression of cellular transcripts and thus prevent synthesis of proteins that contribute directly or indirectly to IgE-mediated diseases Inhibition of target gene expression using RNAi represents a fundamentally new therapeutic approach.

The inventors have selected a specific set of target genes for inhibition that they have identified as likely to be involved in the pathogenesis of IgE-mediated diseases, from among the multitude of genes that are expressed in cells of the immune system. One aspect of the invention is the recognition that inhibiting expression of one or more genes in this set, preferably at the level of RNA transcription, will be of significant benefit. The inventors have also designed novel RNAi agents based on the sequences of preferred target genes. In addition, the inventors have discovered that RNAi agents can effectively inhibit gene expression in the respiratory system of a subject when delivered either directly to the respiratory system or when delivered intravenously. The inventors have further discovered that certain delivery agents markedly and unexpectedly enhance the efficacy of RNAi agents in animal models when used to deliver the agents either directly to the respiratory system or intravenously.

The invention provides RNAi agents targeted to any of a variety of transcripts that encode molecules implicated in the development, pathogenesis, and/or symptomatology of asthma and/or allergic rhinitis. In various embodiments, the invention provides compositions containing short interfering RNA (siRNA) and/or short hairpin RNA (shRNA) targeted to one or more target transcripts involved either directly or indirectly in mast cell or basophil activity and/or in the production of IgE by B cells. In certain embodiments of the invention the siRNA comprises two RNA strands having a region of complementarity approximately 19 nucleotides in length, but ranging in length between 17 and 29 nucleotides, and optionally further comprises one or two single-stranded overhangs. In certain embodiments of the invention the shRNA comprises a single RNA molecule having a region of self-complementarity. The single RNA strand forms a hairpin structure comprising a stem and loop and, optionally, one or more unpaired portions at the 5′ and/or 3′ end of the RNA. Such RNA species are said to self-hybridize.

In addition, the invention provides vectors whose presence within a cell results in transcription of one or more RNAs that self-hybridize or hybridize to each other to form an shRNA or siRNA that inhibits expression of at least one target transcript involved in mast cell or basophil activity and/or in the production of IgE by B cells.

The invention further provides compositions, e.g., pharmaceutical compositions, comprising the inventive RNAi agents (siRNAs, shRNAs, and/or vectors, and methods of delivery of such compositions. For example, the invention provides a vector comprising a nucleic acid operably linked to expression signals (e.g., a promoter or promoter/enhancer) active in a cell so that, when the construct is introduced into the cell, an siRNA or shRNA is produced inside the host cell that is targeted to a target transcript, which transcript is involved in involved either directly or indirectly in mast cell or basophil activity and/or in the production of IgE by B cells. In general, the vector may be a DNA or RNA plasmid or a virus vector such as a retrovirus (e.g., a lentivirus), adenovirus, adeno-associated virus, herpes virus, vaccinia virus, etc. whose presence within a cell results in transcription of one or more ribonucleic acids (RNAs) that self-hybridize or hybridize to each other to form a short hairpin RNA (shRNA) or short interfering RNA (siRNA) that inhibits expression of at least one target transcript in the cell, which transcript is involved either directly or indirectly in mast cell or basophil activity and/or in the production of IgE by B cells. In certain embodiments of the invention the vector comprises a nucleic acid segment operably linked to a promoter, so that transcription results in synthesis of an RNA comprising complementary regions that hybridize to form an shRNA targeted to the target transcript. In certain embodiments of the invention the vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. The invention further provides compositions comprising the vector.

Any of the inventive compositions may comprise, in addition to the siRNAs, shRNAs, and/or vectors described herein, one or more substances, referred to as delivery agents, that facilitate delivery and/or uptake of the siRNA, shRNA, or vector. These substances include cationic polymers; peptide molecular transporters including arginine-rich peptoids or peptides and histidine-rich peptides; cationic and neutral lipids; liposomes; certain non-cationic polymers; carbohydrates; and surfactant materials. Suitable delivery agents are described in co-pending U.S. patent application Ser. Nos. 10/674,159 (published as US2004242518) and 10/674,087, published as US2005008617) and PCT applications published as WO2004028471 and WO2004029213, all of which are incorporated herein by reference. The compositions may be administered by a variety of routes including intravenous, inhalation, intranasally, as an aerosol, intraperitoneally, intramuscularly, intradermally, orally, etc. Methods of delivery that target the respiratory system, e.g., intranasal, inhalational, etc., are particularly of interest.

The present invention further provides methods of treating or preventing diseases or conditions associated with IgE-mediated hypersensitivity, e.g., allergic rhinitis and/or asthma, or of providing symptomatic relief by administering compositions containing one or more inventive RNAi agents to a subject at risk of or suffering from these conditions within an appropriate time window prior to, during, or after exposure to a triggering stimulus such as an antigen. The siRNAs and/or shRNAs may be chemically synthesized, produced using in vitro transcription, produced intracellularly, etc. The compositions may be administered by a variety of routes including intravenous, inhalation, intranasally, as an aerosol, intraperitoneally, intramuscularly, intradermally, orally, etc. In certain preferred embodiments of the invention the compositions comprise one or more siRNAs.

The present invention also provides a system for identifying RNAi agents having sequences that are useful for the treatment or prevention of allergic rhinitis and/or asthma and for IgE-mediated disorders generally. For purposes of description, it will be assumed herein that the inventive compositions are to be used for the treatment of allergic rhinitis and/or asthma. However, it is noted that their use is not limited to these conditions. The inventive compositions may be used in the treatment and/or prophylaxis of any of a variety of conditions in which involvement of IgE is implicated, including food allergies, anaphylactic reactions to insect stings or food allergens, parasitic infections, etc. In addition, they may be used for a variety of other purposes in which it is desired to inhibit expression of the target genes, e.g., for research purposes such as to study the genes themselves, to test candidate pharmaceutical agents, etc.

The present invention further provides a system and reagents for analysis and characterization of the pathophysiology of allergic rhinitis, asthma, and other IgE-mediated conditions and of the role of different cell types and molecules in these conditions as well as for studying various biological processes involving mast cells, basophils, dendritic cells, T cells, and B cells.

This application refers to various patents, patent applications, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Goldsby, R. A, et al., Kuby Immunology, 4^{th} ed., W.H. Freeman and Co., New York, 2000; Goodman and Gilman's The Pharmacological Basis of Therapeutics, 10^{th} ed., McGraw-Hill, 2001 and Physician's Desk Reference, 56^{th} ed., ISBN: 1563634112 Medical Economics, 2002.

Unless otherwise defined, all technical and scientific terms used herein have the same meaning as commonly understood by one of ordinary skill in the art to which this invention belongs. Where ranges are given, the endpoints are included in the range. Where various embodiments of the invention are set forth in Markush group language, or in the alternative, it is to be understood that all subsets and individual members of Markush groups and lists are also implicitly set forth even if not explicitly recited herein. Although methods and materials similar or equivalent to those described herein can be used in the practice or testing of the present invention, suitable methods and materials are described below. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWING

FIG. 1 shows the structure of siRNAs observed in the Drosophila system.

FIG. 2 presents a schematic representation of the steps involved in RNA interference in Drosophila.

FIG. 3 shows structures of a variety of exemplary RNAi agents useful in accordance with the present invention.

FIG. 4 presents a representation of an alternative inhibitory pathway (microRNA translational repression pathway), in which the DICER enzyme cleaves a substrate having a base mismatch in the stem to generate an inhibitory product that binds to the 3′ UTR of a target transcript and inhibits translation.

FIG. 5 presents one example of a construct that may be used to transcribe of both strands of an inventive siRNA.

FIG. 6 depicts one example of a construct that may be used to transcribe a single RNA molecule that hybridizes to form an shRNA in accordance with the present invention.

FIG. 7A shows schematic diagrams of HFcεRα-338 and GFP-949 siRNA and their hairpin derivatives/precursors.

FIG. 7B shows tandem arrays of HFcεRα-338H and GFP-949H in two different orders.

FIG. 7C shows pSLOOP III expression vectors. Hairpin precursors of siRNA (i.e., shRNA sequences) are cloned in pSLOOP III vector alone (top), in tandem arrays (middle), or simultaneously with independent promoter and termination sequence (bottom).

### DEFINITIONS

As used herein, the terms approximately or about in reference to a number are generally taken to include numbers that fall within a range of 5% in either direction (greater than or less than) of the number unless otherwise stated or otherwise evident from the context.

The term complementary is used herein in accordance with its art-accepted meaning to refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation).

A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity. For example, AAAAAAAA and TTTGTTAT are 75% complementary since there are 12 nucleotides in complementary base pairs out of a total of 16. Nucleic acids that are at least 70% complementary over a window of evaluation are considered substantially complementary over that window. Specifically, if the window of evaluation is 15-16 nucleotides long, substantially complementary nucleic acids may have 0-3 mismatches within the window; if the window is 17 nucleotides long, substantially complementary nucleic acids may have 0-4 mismatches within the window; if the window is 18 nucleotides long, substantially complementary nucleic acids may have may contain 0-5 mismatches within the window; if the window is 19 nucleotides long, substantially complementary nucleic acids may contain 0-6 mismatches within the window. The number of permissible mismatches increases by one nucleotide for each additional nucleotide present in the window. In certain embodiments the mismatches are not at continuous positions. In certain embodiments the window contains no stretch of mismatches longer than two nucleotides in length. In preferred embodiments a window of evaluation of 15-19 nucleotides contains 0-1 mismatch (preferably 0), and a window of evaluation of 20-29 nucleotides contains 0-2 mismatches (preferably 0-1, more preferably 0).

Gene, as used herein, has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode structural or functional RNA molecules. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

A gene product or expression product is, in general, an RNA transcribed from the gene or a polypeptide encoded by an RNA transcribed from the gene.

The term hybridize, as used herein, refers to the interaction between two complementary nucleic acid sequences. The phrase hybridizes under high stringency conditions describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.; high stringency hybridization (e.g., 6×SSC at about 45° C., followed by one or more washes in 0.2×SSC, 0.1% SDS at 65° C.; and very high stringency hybridization conditions (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based upon various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

Identity refers to the extent to which the sequence of two or more nucleic acids is the same. A degree of identity between two nucleic acids over a window of evaluation may be computed by aligning the nucleic acids in parallel orientation and determining the percentage of positions within the window of evaluation that are occupied by the same nucleotide in each strand, allowing the introduction of gaps in either strand. Typically a degree of identity is determined over a window of evaluation at least 15 nucleotides in length, e.g., 19 nucleotides.

Inappropriate or excessive, as used herein in reference to the expression of a transcript or in reference to the functional activity of a polypeptide or cell refers to expression or activity that either (i) occurs at a level higher than occurs normally in a wild type cell or healthy subject under typical environmental conditions, typically a level that contributes to or causes a detectable result such as a symptom or sign of disease; and/or (ii) occurs in a temporal or spatial pattern that differs from that which occurs normally in a wild type cell or healthy subject under typical environmental conditions, typically in a nammer that contributes to or causes a detectable result such as a symptom or sign of disease. Inappropriate or excessive expression or activity includes expression or activity in a cell type that does not normally exhibit such expression or activity. Whether or not a cell or subject exhibits inappropriate or excessive expression of a transcript or inappropriate or excessive activity of a polypeptide or functional activity may be determined, for example, by comparing the expression or activity either with normal (e.g., wild type) subjects, with historical controls, with previous values in that subject, etc. However, in certain embodiments of the invention expression or activity is considered inappropriate or excessive in a subject even if it falls within the range that is considered normal.

By associated with, characterized by, or featuring excessive or inappropriate expression of a transcript or polypeptide is generally meant that excessive or inappropriate expression of the transcript or polypeptide frequently (e.g., in a majority of instances), typically, or consistently occurs in the presence of the disease or condition. It is not necessary that excessive or inappropriate expression invariably occurs in the presence of the disease or condition, and in fact excessive or inappropriate expression may only occur in a small subset (e.g., less than 5%) of the subjects suffering from the disease or condition). In general, the excessive or inappropriate expression of the transcript or polypeptide, either directly or indirectly causes or contributes to the disease or condition or a symptom thereof. It is noted that whether or not expression or activity is excessive or inappropriate may depend on context. For example, expression of a receptor for a ligand may have no effect in the absence of the ligand while in the presence of the ligand such expression may be deemed excessive or inappropriate if it results in a disease or symptom. In the therapeutic context, the phrases associated with, characterized by, or featuring generally mean that at least one symptom of the condition or disease to be treated is caused, exacerbated, or contributed to by the transcript or encoded polypeptide, such that a reduction in the expression of the transcript or polypeptide will alleviate, reduce, or prevent one or more features or symptoms of the disease or condition.

Isolated, as used herein, means 1) separated from at least some of the components with which it is usually associated in nature; 2) prepared or purified by a process that involves the hand of man; and/or 3) not occurring in nature.

Operably linked, as used herein, refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Purified, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The term regulatory sequence is used herein to describe a region of nucleic acid sequence that directs, enhances, or inhibits the expression (particularly transcription, but in some cases other events such as splicing or other processing) of sequence(s) with which it is operatively linked. The term includes expression signals such as promoters, enhancers, etc., and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptor subunit genes (see, e.g., Winoto et al., EMBO J. 8:729, 1989) and immunoglobulin genes (see, for example, Banerji et al., Cell 33:729, 1983; Queen et al., Cell 33:741, 1983); and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention the regulatory sequence may comprise a promoter and/or enhancer that is active in epithelial cells in the nasal passages, respiratory tract and/or the lungs. For example, a promoter for a gene that encodes a surfactant protein can be used

As used herein, the term RNAi agent encompasses RNA molecules and vectors (other than naturally occurring molecules not modified by the hand of man) whose presence within a cell results in RNAi and leads to reduced expression of a transcript to which the RNAi agent is targeted. The term specifically includes siRNA, shRNA, and RNAi vectors. The term is used synonymously with the term “RNAi-inducing entity” as used in U.S. Ser. No. 60/549,070.

As used herein, an RNAi vector is a vector whose presence within a cell results in transcription of one or more RNAs that self-hybridize or hybridize to each other to form an shRNA or siRNA. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an shRNA or siRNA. In general, the vector comprises a nucleic acid operably linked to expression signal(s) so that one or more RNA molecules that hybridize or self-hybridize to form an siRNA or shRNA are transcribed when the vector is present within a cell. Thus the vector comprises a template for intracellular synthesis of the RNA or RNAs, or precursors thereof. For purposes of mediating RNAi, presence of a viral genome in a cell (e.g., following fusion of the viral envelope with the cell membrane) is considered sufficient to constitute presence of the virus within the cell. In addition, for purposes of mediating RNAi, a vector is considered to be present within a cell if it is introduced into the cell, enters the cell, or is inherited from an ancestral cell, regardless of whether it is subsequently modified or processed within the cell or within an ancestral cell. An RNAi vector is considered to be targeted to a transcript if presence of the vector within a cell results in production of one or more RNAs that hybridize to each other or self-hybridize to form an siRNA or shRNA that is targeted to the transcript, i.e., if presence of the vector within a cell results in production of one or more siRNAs or shRNAs targeted to the transcript. An RNAi vector can be used to mediate RNAi in a cell that expresses a transcript to which it is targeted, and/or for producing siRNA or shRNA molecules in cells that either do or do not express the transcript. The siRNA or shRNA can be purified from cells that produce it and used for any of the purposes described herein. The term “RNAi vector” is used synonymously with the term “RNAi-inducing vector” as used in U.S. Ser. No. 60/549,070.

A short, interfering RNA (siRNA) comprises an RNA duplex portion that is approximately 15-29 basepairs long and optionally further comprises one or two single-stranded overhangs, e.g., a 3′ overhang on one or both strands. For example, the duplex portion may be 17-19 nucleotides in length or any other subrange or specific value within the interval between 15 and 29, e.g., 19, 21-23, 19-23, 24-27, 27-29. An siRNA may be formed from two RNA molecules that hybridize together, or may alternatively be generated from a single RNA molecule that includes a self-hybridizing portion, as described further below. According to certain embodiments of the invention free 5′ ends of siRNA molecules have phosphate groups, and/or free 3′ ends have hydroxyl groups while according to other embodiments free 5′ ends lack phosphate groups and/or free 3′ ends lack hydroxyl groups. It is generally preferred that free 5′ ends of siRNA molecules have phosphate groups and free 3′ ends have hydroxyl groups. The duplex portion of an siRNA may, but typically does not, contain one or more bulges consisting of one or more unpaired nucleotides. The bulge can be, for example, (i) a mismatch (which occurs when two strands are aligned with each other for maximum complementarity within a window of evaluation and two nucleotides opposite each other in the aligned strands are noncomplementary), or (ii) an area in which one strand contains an “extra” nucleotide with respect to the other strand when the two strands are aligned for maximum complementarity within a window of evaluation; or (iii) a combination of the foregoing.

One strand of an siRNA (which may be referred to as an “antisense strand” or “guide strand” includes a portion that hybridizes with a target transcript. In certain preferred embodiments of the invention, the antisense strand of the siRNA is precisely complementary with a region of the target transcript (100% complementary), meaning that the siRNA hybridizes to the target transcript without a single mismatch or other bulge. In other embodiments of the invention one or more mismatches between the siRNA and the targeted portion of the target transcript may exist. In certain embodiments of the invention in which 100% complementarity is not achieved, it is generally preferred that any mismatches or bulges be located at or near the siRNA termini.

The term short hairpin RNA refers to an RNA molecule comprising at least two complementary portions hybridized or capable of hybridizing to form a double-stranded (duplex) structure sufficiently long to mediate RNAi and at least one single-stranded portion, typically between approximately 1 and 10 nucleotides in length that forms a loop. The duplex region may be 17-19 nucleotides in length or any other subrange or specific value within the interval between 15 and 29, e.g., 19, 21-23, 19-23, 24-27, 27-29. The duplex portion may, but need not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

The term subject, as used herein, refers to any individual susceptible to or suffering from a disease or condition to which IgE is at least in part a causative or contributing factor. The term includes animals, e.g., domesticated animals (such as chickens, swine, horse, dogs, cats, etc.), and wild animals, non-human primates, and humans.

An RNAi agent is considered to be targeted to a target transcript for the purposes described herein if 1) the stability of the target transcript is reduced in the presence of the RNAi agent as compared with its absence; and/or 2) the sequence of the RNAi agent shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) a portion of the agent (e.g., one strand of an siRNA or one of the self-complementary portions of an shRNA) hybridizes to the target transcript under stringent conditions for hybridization of small (<50 nucleotide) RNA molecules in vitro and/or under conditions typically found within the cytoplasm or nucleus of mammalian cells. An RNAi vector whose presence within a cell results in production of an siRNA or shRNA that is targeted to a transcript is also considered to be targeted to the target transcript. Since the effect of targeting a transcript is to reduce or inhibit expression of the gene that comprises a template for synthesis of the transcript, an RNAi agent targeted to a transcript is also considered to target that gene. Thus as used herein, an RNAi agent that targets a transcript is understood to target the gene that provides a template for synthesis of the transcript.

As used herein, treating can generally include one or more of the following: reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur.

In general, the term vector refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules although RNA plasmids are also known), cosmids, and viral vectors. As is well known in the art, the term viral vector may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

### DETAILED DESCRIPTION OF CERTAIN PREFERRED EMBODIMENTS OF THE INVENTION

### I. Overview

The present invention provides compositions comprising RNAi agents such as siRNA(s), shRNA(s), and/or RNAi vectors targeted to transcripts transcribed from one or more gene(s) involved in an IgE-mediated disease or condition, or that encode expression products important for the survival, proliferation, and/or at least one biological activity of cell(s) that are involved in the secretion and/or response to IgE. Any of these transcripts are appropriate targets for RNAi-mediated inhibition in accordance with the present invention. The biological activity can be any activity of the cell including, but not limited to, survival; proliferation; synthesis, secretion, degranulation (e.g., of an inflammatory mediator); migration; cell-cell interaction; etc.

IgE-mediated degranulation of mast cells plays a role in both allergic rhinitis and asthma. Exposure to an allergen activates B cells to form IgE secreting plasma cells. The secreted IgE molecules bind to IgE-specific Fc receptors on basophils in the blood and on mast cells. During the response to subsequent exposure to the allergen, mast cell-associated IgE molecules bind to the allergen, causing crosslinking of the bound IgE and the receptor to which the IgE molecule is bound, which triggers mast cell degranulation. Degranulation involves release of mediators such as histamine that are the proximal cause of the smooth muscle and vascular changes that underlie allergic symptoms. Binding of IgE to mast cells, with subsequent degranulation and release of mediators, is also responsible for many of the symptoms of asthma. See Goldsby, R., Kubyy Immunology, 4^{th} Ed., W.H. Freeman, 2000; Umetsu, D., et al., Nature Immunology, 3(8): 715-720, 2002, for discussion of the pathogenesis of IgE-mediated allergy and asthma.

According to certain embodiments of the present invention RNAi agents are used to inhibit at least one biological activity of mast cells, or to reduce their number or eliminate them (e.g., in the respiratory tract) and/or to inhibit the production of IgE by B cells. The biological activity can be any activity of the cell including, but not limited to, survival, proliferation, synthesis, secretion, migration, cell-cell interaction, etc. In certain preferred embodiments inhibition of the biological activity “inactivates” mast cells, so that they do not exert a pathogenic effect. The invention provides RNAi agents targeted to transcripts encoding a number of different proteins and methods of using them in the treatment of IgE-mediated diseases, including allergy and asthma. Certain preferred embodiments of the invention are described in detail below. It is noted that although the IgE-mediated conditions discussed herein may be referred to as “IgE-mediated hypersensitivity” or “hypersensitivity reactions”, the response of the subject to IgE need not be heightened. In general, the terms may refer to any undesirable or inappropriate inflammatory response to an allergen.

In general, a transcript described as an “X transcript” (e.g., a “CD40 transcript”) means a transcript that is transcribed from the “X gene” (e.g., the CD40 gene), i.e., the gene from which an mRNA that encodes the CD40 protein is transcribed in the appropriate cell type(s). It is noted that although a transcript may be referred to as “encoding” a particular protein, the region of the transcript that is targeted by the RNAi agent need not consist entirely or even in part of a coding portion of the transcript. The targeted region may be, for example, a 5′ or 3′ untranslated region or intron in various embodiments of the invention.

### II. RNAi and Design of RNAi Agents

Whatever gene target is selected, the design of RNAi agents such as siRNAs and shRNAs of the present invention will preferably follow certain guidelines. In general, it is desirable to target sequences that are specific to the transcript whose inhibition is desired. Also, in many cases, the agent that is delivered to a cell or subject according to the present invention may undergo one or more processing steps before becoming an active suppressing agent (see below for further discussion); in such cases, those of ordinary skill in the art will appreciate that the relevant agent will preferably be designed to include sequences that may be necessary for its processing.

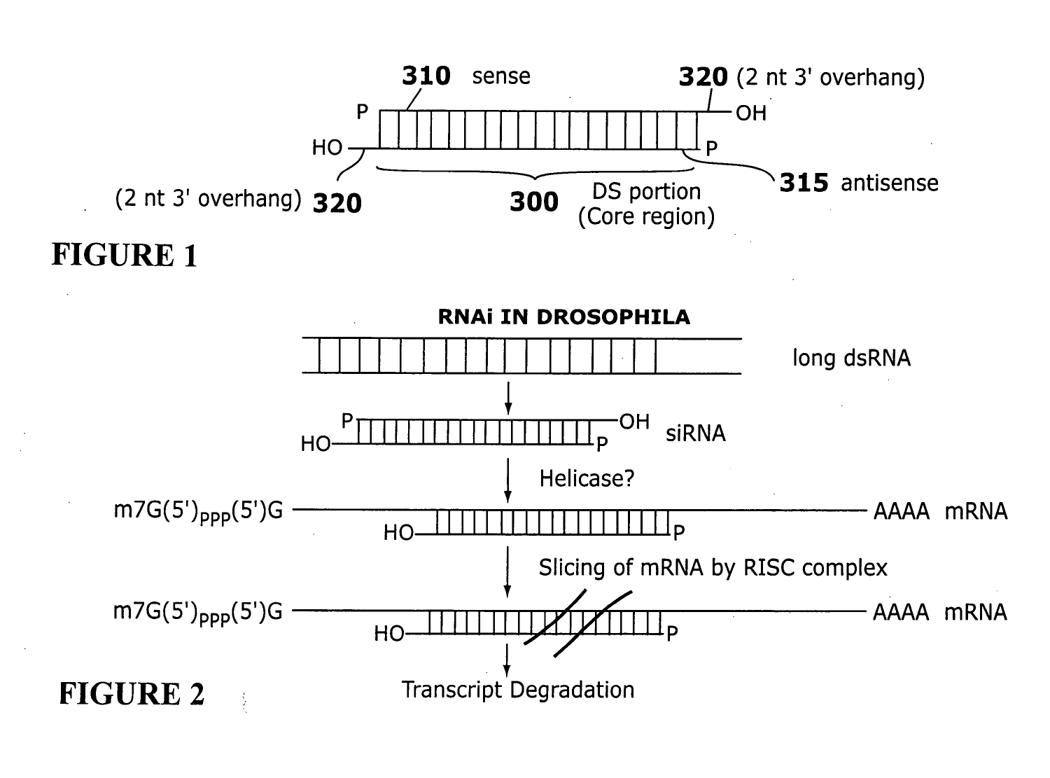

Small inhibitory RNAs were first discovered in studies of the phenomenon of RNA interference (RNAi) in Drosophila, as described in WO 01/75164. In particular, it was found that, in Drosophila, long double-stranded RNAs are processed by an RNase III-like enzyme called DICER (Bernstein et al., Nature 409:363, 2001) into smaller dsRNAs comprised of two 21 nucleotide (nt) strands, each of which has a 5′ phosphate group and a 3′ hydroxyl, and includes a 19 nt region precisely complementary with the other strand, so that there is a 19 nt duplex region flanked by 2 nt-3′ overhangs. FIG. 1 shows a schematic of siRNAs found in Drosophila. The structure includes a 19 nucleotide double-stranded (DS) portion 300, comprising a sense strand 310 and an antisense strand 315. Each strand has a 2 nt 3′ overhang 320.

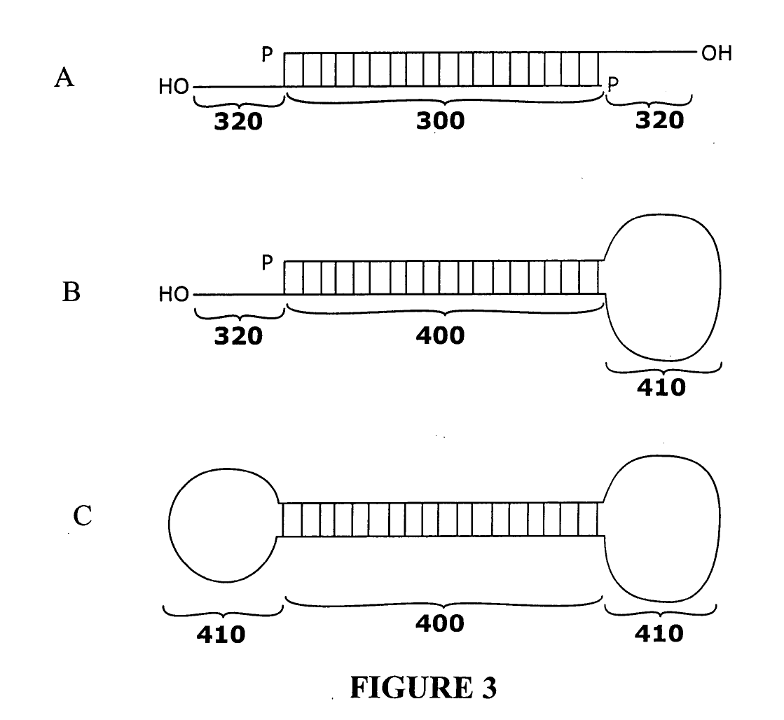

These small dsRNAs (siRNAs) act to silence expression of any gene that includes a region complementary to one of the dsRNA strands, presumably because a helicase activity unwinds the 19 by duplex in the siRNA, allowing an alternative duplex to form between one strand of the siRNA and the target transcript. This new duplex then guides an endonuclease complex, RISC, to the target RNA, which it cleaves (“slices”) at a single location, producing unprotected RNA ends that are promptly degraded by cellular machinery (FIG. 2). As mentioned below, additional mechanisms of silencing mediated by short RNA species (microRNAs) are also known (see, e.g., Ruvkun, G., Science, 294, 797-799, 2001; Zeng, Y., et al., Molecular Cell, 9, 1-20, 2002). The discussion of mechanisms and the figures depicting them are not intended to suggest any limitations on the mechanism of action of the present invention. Further discussion of RNAi is found in Dykxhoorn, D., et al., Nature Reviews Molecular Cell Biology, 4:457-467.

The discovery that homologs of the DICER enzyme occur in diverse species ranging from E. coli to humans (Sharp, Genes Dev. 15; 485, 2001; Zamore, Nat. Struct. Biol. 8:746, 2001), raised the possibility that an RNAi-like mechanism might be able to silence gene expression in a variety of different cell types including mammalian, or even human, cells. Unfortunately, however, long dsRNAs (e.g., dsRNAs having a double-stranded region longer than about 30 nucleotides) are known to activate the interferon response in mammalian cells. Thus, rather than achieving specific gene silencing, introduction of long dsRNAs into mammalian cells would be expected to lead to interferon-mediated non-specific suppression of translation, potentially resulting in cell death. Long dsRNAs are therefore not thought to be useful for specifically inhibiting expression of particular genes in mammalian cells.

However, it has been found that siRNAs, when introduced into mammalian cells, can effectively reduce the expression of target genes. As described in copending patent applications U.S. Ser. Nos. 10/674,159 and 10/674,087, the inventors have shown that siRNAs and/or shRNAs targeted to a variety of transcripts, including both endogenous transcripts such as CD8α and also viral transcripts, greatly reduced the level of the target transcript in mammalian cells. The inventors have also shown that various RNAi agents can inhibit expression of influenza viral transcripts both in mammalian cells in tissue culture, in chick embryos, and in intact animals (mice). Thus treatment with RNAi agents is an effective strategy for reducing or inhibiting the expression of target transcripts. In particular, the inventors have demonstrated that expression of target transcripts can be inhibited in the respiratory passages (e.g., lungs) of intact living animals using various delivery agents and methods for delivery of RNAi agents, thereby establishing the feasibility of using RNAi to treat diseases and conditions that affect the respiratory passages, such as asthma. It is noted that effective inhibition of target transcripts was achieved without the use of hydrodynamic transfection.

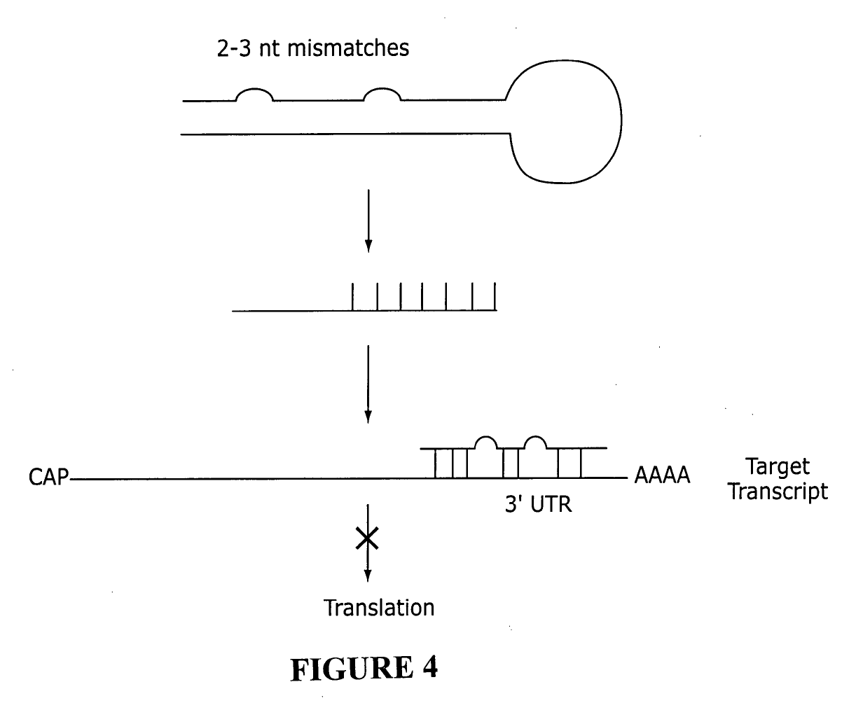

Preferred siRNAs and shRNAs for use in accordance with the present invention include a base-paired region (referred to as a duplex portion or duplex region) between 15-29 nucleotides in length, e.g., approximately 19 nucleotides in length, and may optionally have free or looped ends. For example, FIG. 3 presents various structures that can be utilized in the present invention. FIG. 3A shows the structure found to be active in the Drosophila system described above, which also represents a species that is active in mammalian cells. The present invention encompasses administration of an siRNA having the structure depicted in FIG. 3A to mammalian cells in order to treat or prevent IgE-mediated diseases and conditions including, but not limited to, allergic rhinitis and asthma. However, it is not required that the administered agent have this structure. For example, the administered composition may include any structure capable of being processed in vivo to the structure of FIG. 3A, so long as the administered agent does not lead to negative events such as induction of the interferon response. (Note that the term in vivo, as used herein with respect to the synthesis, processing, or activity of siRNA or shRNA, generally refers to events that occur within a cell as opposed to in a cell-free system. In general, the cell can be maintained in tissue culture or can be part of an intact organism.) The invention may also comprise administration of agents that are not processed to precisely the structure depicted in FIG. 3A, so long as administration of such agents reduces target transcript levels sufficiently as discussed herein. FIGS. 3B and 3C present two alternative structures for use as RNAi agents in the present invention.

FIGS. 3B and 3C represent additional structures that may be used to mediate RNA interference. These hairpin (stem-loop) structures may be processed intracellularly to yield an siRNA structure such as that depicted in FIG. 3A. FIG. 3B shows an agent (shRNA) comprising an RNA molecule containing two complementary portions that hybridize to one another to form a duplex region represented as stem 400, a loop 410, and an overhang 320. Preferably, the stem is between 15-29 nucleotides in length, e.g., approximately 19 nt long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. One of ordinary skill in the art will appreciate that loops of 4 nucleotides or greater are less likely subject to steric constraints than are shorter loops and therefore may be preferred. In some embodiments, the overhang includes a 5′ phosphate or 3′ hydroxyl. As discussed below, an agent having the structure depicted in FIG. 3B can readily be generated by in vivo or in vitro transcription; in several preferred embodiments, the transcript tail will be included in the overhang, so that often the overhang will comprise a plurality of U residues, e.g., between 1 and 5 U residues. The loop may be located at either the 5′ or 3′ end of the portion that is complementary to the target transcript whose inhibition is desired (i.e., the antisense portion of the shRNA).

FIG. 3C shows an agent comprising an RNA circle that includes complementary elements sufficient to form a stem 400 approximately 19 by long. Such an agent may show improved stability as compared with various other RNAi agents described herein.

In describing siRNAs it is often convenient to refer to sense and antisense strands of the siRNA. In general, the sequence of the duplex portion of the sense strand of the siRNA is substantially identical to the targeted portion of the target transcript, while the antisense strand of the siRNA is substantially complementary to the target transcript in this region as discussed further below. Although shRNAs contain a single RNA molecule that self-hybridizes, it will be appreciated that the resulting duplex structure may be considered to comprise sense and antisense strands or portions. It will therefore be convenient herein to refer to sense and antisense strands, or sense and antisense portions, of an shRNA, where the antisense strand or portion is that segment of the molecule that forms or is capable of forming a duplex and is substantially complementary to the targeted portion of the target transcript, and the sense strand or portion is that segment of the molecule that forms or is capable of forming a duplex and is substantially identical in sequence to the targeted portion of the target transcript.

For purposes of description, the discussion below may refer to siRNA rather than to siRNA or shRNA. However, as will be evident to one of ordinary skill in the art, teachings relevant to the sense and antisense strand of an siRNA are generally applicable to the sense and antisense portions of the stem portion of a corresponding shRNA. Thus in general the considerations below apply also to the design, selection, and delivery of inventive shRNAs.

It will be appreciated by those of ordinary skill in the art that agents having any of the structures depicted in FIG. 3, or any other effective structure as described herein, may be comprised entirely of natural RNA nucleotides, or may instead include one or more nucleotide analogs. A wide variety of such analogs are known in the art; the most commonly-employed in studies of therapeutic nucleic acids being the phosphorothioate (for some discussion of considerations involved when utilizing phosphorothioates, see, for example, Agarwal, Biochim. Biophys. Acta 1489:53, 1999). In particular, in certain embodiments of the invention it may be desirable to stabilize the siRNA structure, for example by including nucleotide analogs at one or more free strand ends in order to reduce digestion, e.g., by exonucleases. The inclusion of deoxynucleotides, e.g., pyrimidines such as deoxythymidines at one or more free ends may serve this purpose. Alternatively or additionally, it may be desirable to include one or more nucleotide analogs in order to increase or reduce stability of the stem, in particular as compared with any hybrid that will be formed by interaction of one strand of the siRNA (or one strand of the stem portion of the shRNA) with a target transcript.

According to certain embodiments of the invention various nucleotide modifications are used selectively in either the sense or antisense strand. For example, it may be preferable to utilize unmodified ribonucleotides in the antisense strand while employing modified ribonucleotides and/or modified or unmodified deoxyribonucleotides at some or all positions in the sense strand. According to certain embodiments of the invention only unmodified ribonucleotides are used in the duplex portion of the antisense and/or the sense strand of the siRNA while the overhang(s) of the antisense and/or sense strand may include modified ribonucleotides and/or deoxyribonucleotides. In particular, according to certain embodiments of the invention the sense strand contains a modification that reduces or eliminates silencing of transcripts complementary to the sense strand while not preventing silencing of transcripts complementary to the antisense strand, as described in co-pending U.S. patent application Ser. No. 10/674,159.

Numerous nucleotide analogs and nucleotide modifications are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) “Antisense Drug Technology: Principles, Strategies, and Applications” (1^{st} ed), Marcel Dekker; ISBN: 0824705661; 1st edition (2001) and references therein. As will be appreciated by one of ordinary skill in the art, analogs and modifications may be tested using, e.g., the assays described herein or other appropriate assays, in order to select those that effectively reduce expression of viral genes.

In certain embodiments of the invention the analog or modification results in an siRNA with increased absorbability (e.g., increased absorbability across a mucus layer, increased absorption, etc.), increased stability in the blood stream or within cells, increased ability to cross cell membranes, etc. As will be appreciated by one of ordinary skill in the art, analogs or modifications may result in altered Tm, which may result in increased tolerance of mismatches between the siRNA sequence and the target while still resulting in effective suppression.

It will further be appreciated by those of ordinary skill in the art that effective siRNA agents for use in accordance with the present invention may comprise one or more moieties that is/are not nucleotides or nucleotide analogs.

In general, inventive siRNAs and shRNAs will preferably include a region (the “inhibitory region” or “duplex region”) that contains a strand (the “guide” or “antisense” strand) that is substantially complementary to a portion of the target transcript (target portion), so that a precise hybrid can form in vivo between this strand and the target transcript. In certain preferred embodiments of the invention, the antisense strand of the siRNA or shRNA is perfectly (100%) complementary to the target transcript; in other embodiments, one or more non-complementary residues are located within the duplex formed by the siRNA or shRNA antisense strand and the target transcript. It may be preferable to avoid mismatches in the central portion of this duplex (see, for example, Elbashir et al., EMBO J. 20:6877, 2001, incorporated herein by reference). In general, the antisense strand of the siRNA is substantially complementary to the targeted portion of the target transcript, while the sequence of the sense strand of the siRNA or shRNA is substantially complementary to the antisense strand. Typically, therefore, the sense strand contains a portion that is substantially identical to the targeted portion of the target transcript. However, one of ordinary skill in the art will appreciate that the percent complementarity exhibited by the duplex formed between the antisense strand and the target portion need not be the same as the percent complementarity exhibited by the duplex formed between the sense and antisense strands of the siRNA or shRNA (the inhibitory region).

In certain preferred embodiments of the invention, the siRNA or shRNA antisense strand hybridizes with a target portion that includes exonic sequences in the target transcript. Hybridization with intronic sequences is not excluded, but generally appears not to be preferred in mammalian cells. In certain preferred embodiments of the invention, the siRNA or shRNA antisense strand hybridizes exclusively with exonic sequences. In some embodiments of the invention, the siRNA or shRNA antisense strand hybridizes with a target portion that includes only sequences within a single exon; in other embodiments the target portion is created by splicing or other modification of a primary transcript. Any target region that is available for hybridization with an siRNA or shRNA strand, resulting in slicing and degradation of the transcript, may be utilized in accordance with the present invention. Nonetheless, those of ordinary skill in the art will appreciate that, in some instances, it may be desirable to select particular regions of target gene transcript as siRNA or shRNA hybridization targets. For example, it may be desirable to avoid sections of target gene transcript that may be shared with other transcripts whose degradation is not desired. Coding regions and regions closer to the 3′ end of the transcript than to the 5′ end are preferred in certain embodiments of the invention.

siRNA and shRNA sequences may be selected according to a variety of approaches. As mentioned above, siRNAs and shRNAs preferably include a region (the “duplex region”) comprising an antisense strand that is substantially complementary or, preferably perfectly complementary, to a portion of the target transcript (the “target portion”), so that a hybrid can form in vivo between this strand and the target transcript, and a sense strand comprising a portion that is substantially or perfectly complementary to the antisense strand. The duplex region, also referred to as the “core region” is understood not to include 3′ overhangs, although overhangs, if present, may also be complementary to the target transcript or its complement (e.g., the 3′ overhang of the antisense siRNA (or shRNA) strand may be complementary to the target transcript and the 3′ overhang of the sense siRNA (or shRNA) strand may be identical to the corresponding nucleotides in the target transcript, i.e., those nucleotides immediately 3′ of the target site). While it is generally preferred that the siRNA or shRNA antisense strand is perfectly complementary to the target portion and that the siRNA and shRNA antisense strands are perfectly complementary to one another within the portions that participate in formation of the duplex region, less than perfect complementarity is acceptable and in certain embodiments of the invention is desirable. siRNAs and shRNAs comprising an antisense strand that is less than 100% complementary to the target transcript can mediate RNAi. In addition, siRNA and shRNA comprising antisense and sense strands that are less than perfectly complementary to one another within the core region can also mediate RNAi.

For purposes of description herein, the length of an siRNA or shRNA core region will be assumed to be 19 nucleotides, and a 19 nucleotide sequence is referred to as N19. However, the core region may range in length from 15 to 29 nucleotides. Typically the length of each of the two strands is approximately between 21 and 25 nucleotides although other lengths are also acceptable. Typically the overhangs, if present, are 2 nucleotides in length, although they may be 1 nucleotide or longer than 2 nucleotides. In addition, it is assumed that the siRNA N19 inhibitory region will be chosen so that the portion of the antisense strand that is complementary to the target transcript is perfectly complementary to the target transcript, though as mentioned above one or more mismatches may be tolerated.

In general it is desirable to avoid mismatches in the duplex region if an siRNA having maximal ability to reduce expression of the target transcript via the transcriptional inhibition pathway (transcript cleavage pathway) is desired. However, as described below, it may be desirable to select an siRNA or shRNA that exhibits less than maximal ability to reduce expression of the target transcript, or it may be desirable to employ an siRNA that acts via an alternative pathway involving translational repression. In such situations it may be desirable to incorporate one or more mismatches in the duplex portion of the siRNA or shRNA. In certain embodiments of the invention preferably fewer than four residues or alternatively less than about 15% of residues in the inhibitory region are mismatched. In certain embodiments of the invention preferably fewer than four residues or alternatively less than about 15% of residues in the portion of the antisense strand that is within the inhibitory region are mismatched.

In some cases the siRNA or shRNA sequence is selected such that the entire antisense strand (including the 3′ overhang if present) is perfectly complementary to the target transcript. In cases where the overhang is UU, TT, or dTdT, this requires that the 19 bp target region of the targeted transcript is preceded by AA (i.e., that the two nucleotides immediately 5′ of the target region are AA). Similarly, the siRNA or shRNA sequence may be selected such that the entire sense strand (including the 3′ overhang) is perfectly identical to the target transcript. In cases where the overhang is UU, TT, or dTdT, this requires that the 19 by target region of the targeted transcript is followed by UU (i.e., that the two nucleotides immediately 3′ of the target region of the target transcript are UU). However, it is not necessary that overhang(s) are either complementary or identical to the target transcript. Any desired sequence (e.g., UU) may simply be appended to the 3′ ends of antisense and/or sense 19 by core regions of an siRNA or shRNA to generate 3′ overhang(s). In general, overhangs containing one or more pyrimidines, usually U, T, or dT, are employed. When chemically synthesizing siRNAs or shRNAs it may be more convenient to use T rather than U, while use of dT rather than T may confer increased stability. As indicated above, the presence of overhangs is optional and, where present, they need not have any relationship to the target sequence itself.

For example, siRNAs and shRNAs may be selected by (i) identifying 23 nt regions in the target transcript consisting of 19 nt regions (target portions) flanked by two AA residues at the 5′ end and two UU residues at the 3′ end and then (ii) selecting siRNAs and shRNAs having an antisense strand perfectly complementary to nucleotides 1-21 of the 23 nt region and a sense strand perfectly identical to nt 3-23 of the 23 nt region. It will be appreciated that where the target transcript is an mRNA, siRNA and shRNA sequences may be selected with reference to the corresponding cDNA sequence rather than to the mRNA sequence itself, since the sense strand of the cDNA is identical to the mRNA except that the cDNA contains T rather than U.

Not all siRNAs and shRNAs are equally effective in reducing or inhibiting expression of any particular target gene. (See, e.g., Holen, T., et al., Nucleic Acids Res., 30(8):1757-1766, reporting variability in the efficacy of different siRNAs), and a variety of considerations may be employed to increase the likelihood that a selected siRNA may be effective. For example, it may be preferable to select target portions within exons rather than introns. In general, target portions near the 3′ end of a target transcript may be preferred to target portions near the 5′ end or middle of a target transcript. siRNAs may generally be designed in accordance with principles described in RNAi Technical Reference & Application Guide, available from Dharmacon Research, Inc., Lafayette, Colo. 80026, a commercial supplier of RNA reagents, or in or in Dharmacon Technical Bulletin # 003-Revision B, “siRNA Oligonucleotides for RNAi Applications”. The RNAi Technical Reference & Application Guide contains a variety of information relevant to siRNA and shRNA design parameters, synthesis, etc., and is incorporated herein by reference.

Generally it is preferable to select siRNAs and shRNAs with a GC content between 30% and 60% and to avoid strings of three or more identical nucleotides, e.g., GGG, CCC, etc. In order to achieve specific inhibition of the target transcript while avoiding inhibition of other transcripts, it is desirable to select sequences that are unique or lack significant homology to other sequences present in the cell or organism to which the siRNA or shRNA is delivered, to the extent possible. This may be achieved by searching publicly available databases, e.g., Genbank, draft human genome sequence, etc., to identify any sequences that are homologous to either strand of a proposed siRNA or shRNA sequence and avoiding the use of siRNAs or shRNAs for which one or more substantially identical sequences is found. It may be preferable to select siRNAs or shRNAs that target a portion of the transcript that is identical to or highly conserved (e.g., differing by 3, more preferably 2, or still more preferably 1 nucleotides per 19 nucleotides, and most preferably identical) in the corresponding mouse and human genes. This allows testing of an RNAi agent in mouse cell lines and in mouse models of disease and increases the likelihood that a sequence identified as effective at inhibiting a target gene in mice will also prove effective in inhibiting the corresponding target gene in humans.

Tables 1-26 list sequences of preferred target portions of transcripts encoding FCεR α chain, FCεR β chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2, respectively.

According to certain embodiments of the invention, to design an siRNA based on any of the sequences listed in the tables, nucleotides 1-19 of the sequence is selected as the sequence of the core (duplex) region of the siRNA sense strand, i.e., the portion that will participate in duplex formation. A sequence complementary to this sequence is selected for the antisense strand. A two nt 3′ overhang is added to both the sense and antisense strands. In certain embodiments of the invention the overhang is dTdT although other overhangs may also be used, as described above. In certain embodiments of the invention the 3′ overhang for the sense strand consists of the two nucleotides in the gene immediately 3′ to the 19 nucleotide sequence listed in the table, so that the sense strand is identical to a 21 nt portion of the cDNA sequence (optionally with the replacement of T by U). In certain embodiments of the invention the 3′ overhang for the antisense strand consists of the two nucleotides in the gene complementary to the two nucleotides immediately 5′ to the 19 nucleotide sense strand, so that the antisense strand is complementary to a 21 nt portion of the cDNA sequence. A sequence complementary to nucleotides 1-21 of each sequence is selected as the corresponding antisense strand. For example, to design an siRNA based on the cDNA sequence FCεRα-268 (TTGGTCATTGTGAGTGCCA=SEQ ID NO: 316), the sequence 5′-UUGGUCAUUGUGAGUGCCA-3′ (SEQ ID NO: 1) is selected as the core region of the sense strand, and a complementary sequence, 5′-UGGCACUCACAAUGACCAA-3′ (SEQ ID NO: 317), is selected as the core region of the antisense strand. A two nt 3′ overhang consisting of dTdT is added to each strand, resulting in the sequences 5′-UUGGUCAUUGUGAGUGCCAdTdT-3′ (SEQ ID NO: 318) (sense strand) and 5′-UGGCACUCACAAUGACCAAdTdT-3′ (SEQ ID NO: 319) (antisense strand).

Hybridization of the sense and antisense strands results in an siRNA having a 19 base pair core duplex region, with each strand having a 2 nucleotide 3′ OH overhang. Sense and antisense siRNA sequences may be similarly obtained from each sequence listed in the tables. It will be appreciated that the 19 nt core regions may be used to design a variety of siRNA molecules having different 3′ overhangs in either or both the sense and antisense strands. In general, the invention encompasses siRNAs in which the sense strand includes a highly conserved core region while the 3′ overhangs may vary. The 3′ overhang in the sense strand need not correspond to nucleotides present immediately 3′ of the core region in the cDNA sequence. The 3′ overhang in the antisense strand need not be complementary to the nucleotides immediately 5′ of the core region in the cDNA sequence.

In accordance with the description presented above, the sequences presented in the tables may be used to design a variety of siRNAs that do not have a structure consisting of a 19 nt duplex core region with identical 3′ overhangs on each strand. For example, the sequence of the overhangs may be varied, and the presence of one or both of the overhangs may not be essential for effective siRNA mediated inhibition of gene expression. In addition, although the preferred length of the duplex portion of an siRNA may be 19 nucleotides, shorter or longer duplex portions may be effective. Thus siRNAs designed in accordance with the sequences presented in the tables may include only a subset of the listed nucleotides in the sense strand.

The invention therefore provides siRNAs having sense strands with sequences that include all or a portion of the 19 nucleotides in the sequences listed in the tables. Generally, the sequence of the sense strand of an siRNA designed in accordance with a sequence presented in the tables will include at least 15 consecutive nucleotides, more preferably at least 17 consecutive nucleotides, and yet more preferably 19 consecutive nucleotides of the listed sequence. Generally the sequence of the antisense strand of an siRNA designed in accordance with a sequence presented in the tables will include at least 15 consecutive nucleotides, more preferably at least 17 consecutive nucleotides, and yet more preferably 19 consecutive nucleotides that are perfectly complementary to a portion of the listed sequence.

In certain embodiments of the invention the sequence of the sense strand of an siRNA designed in accordance with a sequence presented in the tables will include at least 15 consecutive nucleotides, more preferably at least 17 consecutive nucleotides, and yet more preferably 19 consecutive nucleotides of the listed sequence, with one nucleotide difference from the listed sequence. In certain embodiments of the invention the sequence of the antisense strand of an siRNA designed in accordance with a sequence presented in the tables will include at least 15 consecutive nucleotides, more preferably at least 17 consecutive nucleotides, and yet more preferably 19 consecutive nucleotides that are perfectly complementary to a portion of the listed sequence except that one nucleotide may differ.

In certain embodiments of the invention the sequence of the sense strand of an siRNA designed in accordance with a sequence presented in the tables will include at least 15 consecutive nucleotides, more preferably at least 17 consecutive nucleotides, and yet more preferably 19 consecutive nucleotides of the listed sequence, with up to two nucleotides different from the listed sequence. In certain embodiments of the invention the sequence of the antisense strand of an siRNA designed in accordance with a sequence presented in the tables will include at least 15 consecutive nucleotides, more preferably at least 17 consecutive nucleotides, and yet more preferably 19 consecutive nucleotides that are perfectly complementary to a portion of the listed sequence except that two nucleotides may differ.

In certain embodiments of the invention the sequence of the sense strand of an siRNA designed in accordance with a sequence presented in the tables will include at least 15 consecutive nucleotides, more preferably at least 17 consecutive nucleotides, and yet more preferably 19 consecutive nucleotides of the listed sequence, with up to three nucleotides different from the listed sequence. In certain embodiments of the invention the sequence of the antisense strand of an siRNA designed in accordance with a sequence presented in the tables will include at least 15 consecutive nucleotides, more preferably at least 17 consecutive nucleotides, and yet more preferably 19 consecutive nucleotides that are perfectly complementary to a portion of the listed sequence except that three nucleotides may differ. In any of these embodiments, differences can be an insertion, deletion, or substitution of one nucleotide or, in certain embodiments of the invention, more than one nucleotide, with respect to the original sequence. The invention provides shRNAs having sense and antisense strands as described above for siRNA. siRNA or shRNA antisense and sense strands sequences having differences as described above are considered substantially complementary or substantially identical to the listed sequences, respectively.

One of ordinary skill in the art will appreciate that siRNAs may exhibit a range of melting temperatures (Tm) and dissociation temperatures (Td) in accordance with the foregoing principles. The Tm is defined as the temperature at which 50% of a nucleic acid and its perfect complement are in duplex in solution while the Td, defined as the temperature at a particular salt concentration, and total strand concentration at which 50% of an oligonucleotide and its perfect filter-bound complement are in duplex, relates to situations in which one molecule is immobilized on a filter. Representative examples of acceptable Tms may readily be determined using methods well known in the art, either experimentally or using appropriate empirically or theoretically derived equations, based on the siRNA sequences disclosed in the Examples herein.

One common way to determine the actual Tm is to use a thermostatted cell in a UV spectrophotometer. If temperature is plotted vs. absorbance, an S-shaped curve with two plateaus will be observed. The absorbance reading halfway between the plateaus corresponds to Tm. The simplest equation for Td is the Wallace rule: Td=2(A+T)+4(G+C) Wallace, R. B.; Shaffer, J.; Murphy, R. F.; Bonner, J.; Hirose, T.; Itakura, K., Nucleic Acids Res. 6, 3543 (1979). The nature of the immobilized target strand provides a net decrease in the Tm observed relative to the value when both target and probe are free in solution. The magnitude of the decrease is approximately 7-8° C. Another useful equation for DNA which is valid for sequences longer than 50 nucleotides from pH 5 to 9 within appropriate values for concentration of monovalent cations, is: Tm=81.5+16.6 log M+41(XG+XC)−500/L−0.62F, where M is the molar concentration of monovalent cations, XG and XC are the mole fractions of G and C in the sequence, L is the length of the shortest strand in the duplex, and F is the molar concentration of formamide (Howley, P. M; Israel, M. F.; Law, M-F.; Martin, M. A., J. Biol. Chem. 254, 4876). Similar equations for RNA are: Tm=79.8+18.5 log M+58.4 (XG+XC)+11.8(XG+XC)2−820/L−0.35F and for DNA-RNA hybrids: Tm=79.8+18.5 log M+58.4 (XG+XC)+11.8(XG+XC)2−820/L−0.50F.

These equations are derived for immobilized target hybrids. Several studies have derived accurate equations for Tm using thermodynamic basis sets for nearest neighbor interactions. The equation for DNA and RNA is: Tm=(1000ΔH)/A+ΔS+R1n(Ct/4)−273.15+16.6 ln[Na⁺], where ΔH (Kcal/mol) is the sum of the nearest neighbor enthalpy changes for hybrids, A (eu) is a constant containing corrections for helix initiation, ΔS (eu) is the sum of the nearest neighbor entropy changes, R is the Gas Constant (1.987 cal deg⁻¹ mol⁻¹) and Ct is the total molar concentration of strands. If the strand is self complementary, Ct/4 is replaced by Ct. Values for thermodynamic parameters are available in the literature. For DNA see Breslauer, et al., Proc. Natl. Acad. Sci. USA 83, 3746-3750, 1986. For RNA:DNA duplexes see Sugimoto, N., et al, Biochemistry, 34(35): 11211-6, 1995. For RNA see Freier, S. M., et al., Proc. Natl. Acad. Sci. 83, 9373-9377, 1986. Rychlik, W., et al., Nucl. Acids Res. 18(21), 6409-6412, 1990. Various computer programs for calculating Tm are widely available. See, e.g., the Web site having URL www.basic.nwu.edu/biotools/oligocalc.html. According to certain embodiments of the invention, preferred siRNAs are selected in accordance with the design criteria described in Semizarov, D., et al., Proc. Natl. Acad. Sci., 100(11), pp. 6347-6352.

In some embodiments of the invention, the siRNA or shRNA antisense strand hybridizes to a target site that includes one or more 3′ UTR sequences or is completely within the 3′ UTR. Such embodiments of the invention may tolerate a larger number of mismatches in the siRNA/template duplex, and particularly may tolerate mismatches within the central region of the duplex. In fact, some mismatches may be desirable as siRNA/template duplex formation in the 3′ UTR may inhibit expression of a protein encoded by the template transcript by a mechanism related to but distinct from classic RNA inhibition. In particular, there is evidence to suggest that siRNAs that bind to the 3′ UTR of a template transcript may reduce translation of the transcript rather than decreasing its stability. For example, when hybridized with the target transcript such siRNAs frequently include two stretches of perfect complementarity separated by a region of mismatch. A variety of structures are possible. For example, the siRNA or shRNA, and/or the duplex formed by the siRNA or shRNA antisense strand and the target transcript, may include one or multiple areas of less than perfect complementarity (e.g., mismatched nucleotides, bulges, etc.). Typically the stretches of perfect complementarity are at least 5 nucleotides in length, e.g., 6, 7, or more nucleotides in length, while the regions of mismatch may be, for example, 1, 2, 3, or 4 nucleotides in length.

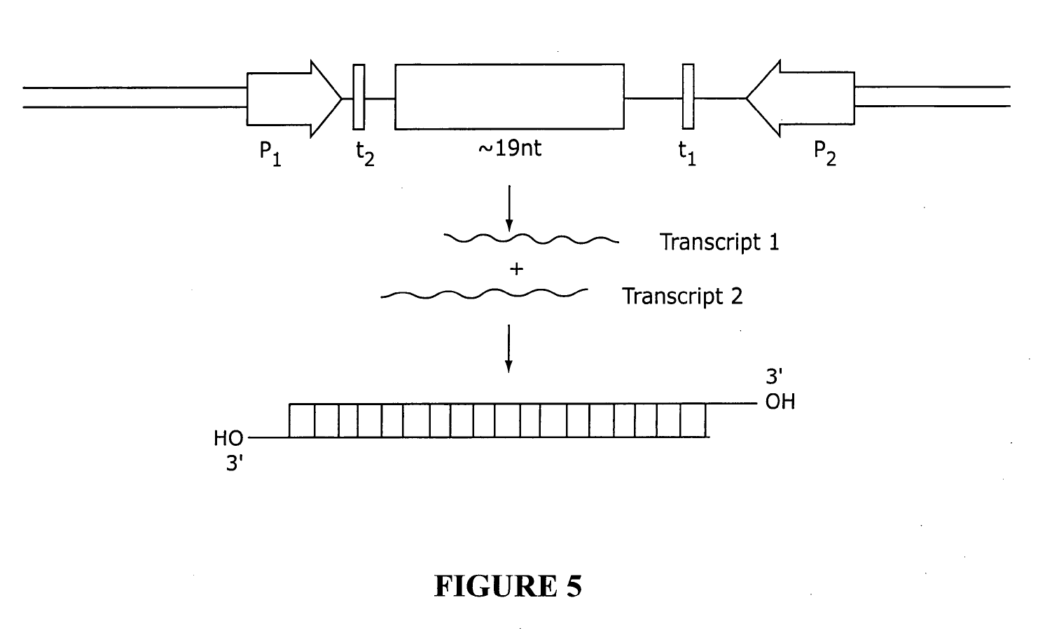

Short double-stranded RNAs comprising an antisense strand that displays less than perfect complementarity to a target transcript may silence gene expression by translational repression in addition to, or instead of, by leading to cleavage of the target transcript. For example, as shown in FIG. 4, the DICER enzyme that generates siRNAs in the Drosophila system discussed above and also in a variety of organisms, is known to also be able to process a small, temporal RNA (stRNA) substrate into an inhibitory agent that, when bound within the 3′ UTR of a target transcript, blocks translation of the transcript (see FIG. 4; Grishok, A., et al., Cell 106, 23-24, 2001; Hutvagner, G., et al., Science, 293, 834-838, 2001; Ketting, R., et al., Genes Dev., 15, 2654-2659. Similar ˜22 nucleotide RNAs, generally referred to as microRNAs (miRNAs) have been identified in a number of organisms including mammals, suggesting that this mechanism of post-transcriptional gene silencing may be widespread (Lagos-Quintana, M. et al., Science, 294, 853-858, 2001; Pasquinelli, A., Trends in Genetics, 18(4), 171-173, 2002, and references in the foregoing two articles). MicroRNAs are transcribed as ˜70 nt precursor hairpin RNAs containing an ˜4-15 nt loop and, typically, one or more areas of mismatch or bulges in the stem. MicroRNAs processed from such precursors have been shown to block translation of target transcripts containing target sites in mammalian cells (Zeng, Y., et al., Molecular Cell, 9, 1-20, 2002) although they can also recognize their targets and direct RNA cleavage (Hutvagner, G. and Zamore, P. D., Science, 297: 2056-2060, 2002; Zeng, Y., et al., Mol. Cell. 9: 1327-1333, 2002). Ambros, V., et al. have proposed a uniform system for microRNA annotation and for distinguishing between endogenous siRNAs and miRNAs (Ambros, V., et al., RNA, 9:277-279, 2003.) See also, Bartel, D., Cell, 116(2):281-97, 2004.

Hairpin structures designed to mimic siRNA and/or miRNA precursors are processed intracellularly into molecules capable of reducing or inhibiting expression of target transcripts (McManus, M. T., et al., RNA, 8:842-850, 2002). These hairpin structures, which were based on classical siRNAs consisting of two RNA strands forming a 100% complementary duplex structure were classified as class I or class II hairpins. Class I hairpins incorporated a loop at the 5′ or 3′ end of the antisense siRNA strand (i.e., the strand complementary to the target transcript whose inhibition is desired) but were otherwise identical to classical siRNAs. Class II hairpins resembled miRNA precursors in that they included a 19 nt duplex region and a loop at either the 3′ or 5′ end of the antisense strand of the duplex in addition to one or more nucleotide mismatches in the stem. These molecules were processed intracellularly into small RNA duplex structures capable of mediating silencing. They appeared to exert their effects through degradation of the target mRNA rather than through translational repression as is thought to be the case for naturally occurring miRNAs. siRNAs having perfectly complementary duplex structures but whose antisense strand formed a less than perfectly complementary duplex with a target (i.e., the duplex contained a bulge), appeared to silence gene expression by inhibiting translation (Doench, J., et al., Genes & Dev., 17:438-442, 2003).

Thus it is evident that RNA molecules containing duplex structures, one portion of which is substantially or perfectly complementary to a target transcript, mediate silencing through at least two different mechanisms. For the purposes of the present invention, any such RNA, one portion of which binds to a target transcript and reduces its expression, whether by triggering degradation, by inhibiting translation, or by other means, is considered to be an RNAi agent and is useful in the practice of the present invention. Any composition or method described herein may be specifically limited to certain RNA structures.

Those of ordinary skill in the art will readily appreciate that inventive RNAi agents may be prepared according to any available technique including, but not limited to chemical synthesis, enzymatic or chemical cleavage in vivo or in vitro, or template transcription in vivo or in vitro. As noted above, inventive RNAi agents may be delivered as a single RNA strand including self-complementary portions (shRNA), or as two (or possibly more) strands hybridized to one another (siRNA). For instance, two separate 21 nt RNA strands may be generated, each of which contains a 19 nt region complementary to the other, and the individual strands may be hybridized together to generate a structure such as that depicted in FIG. 3A.

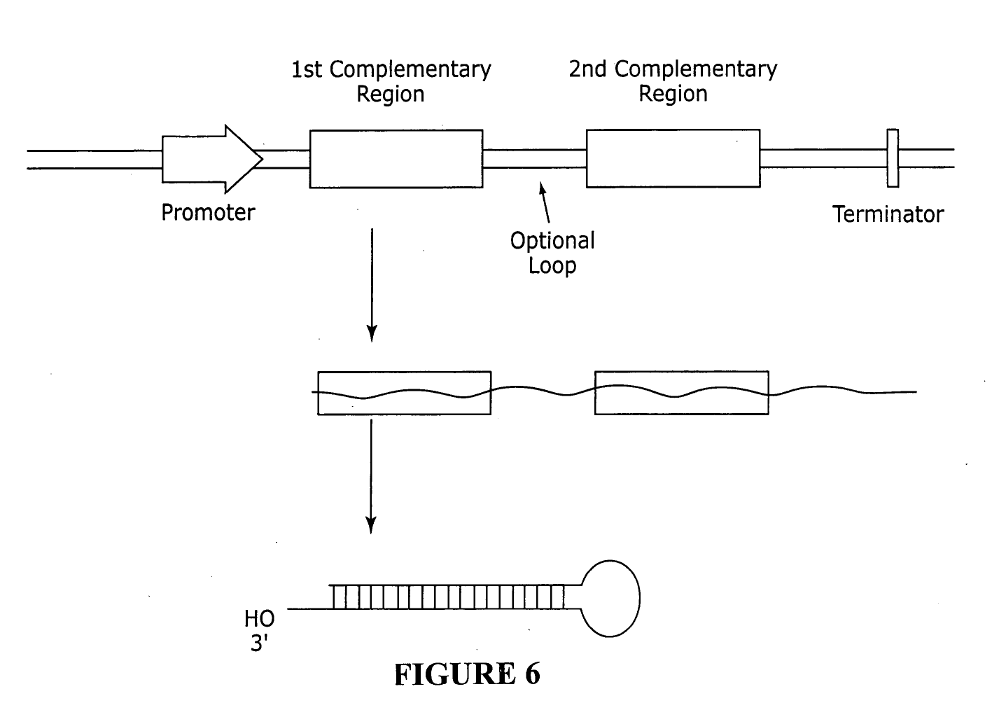

Alternatively, each strand may be generated by transcription from a promoter, either in vitro or in vivo. For instance, a construct may be provided containing two separate transcribable regions, each of which generates a 21 nt transcript containing a 19 nt region complementary with the other. Alternatively, a single construct may be utilized that contains opposing promoters P1 and P2 and terminators t1 and t2 positioned so that two different transcripts, each of which is at least partly complementary to the other, are generated as indicated in FIG. 5.

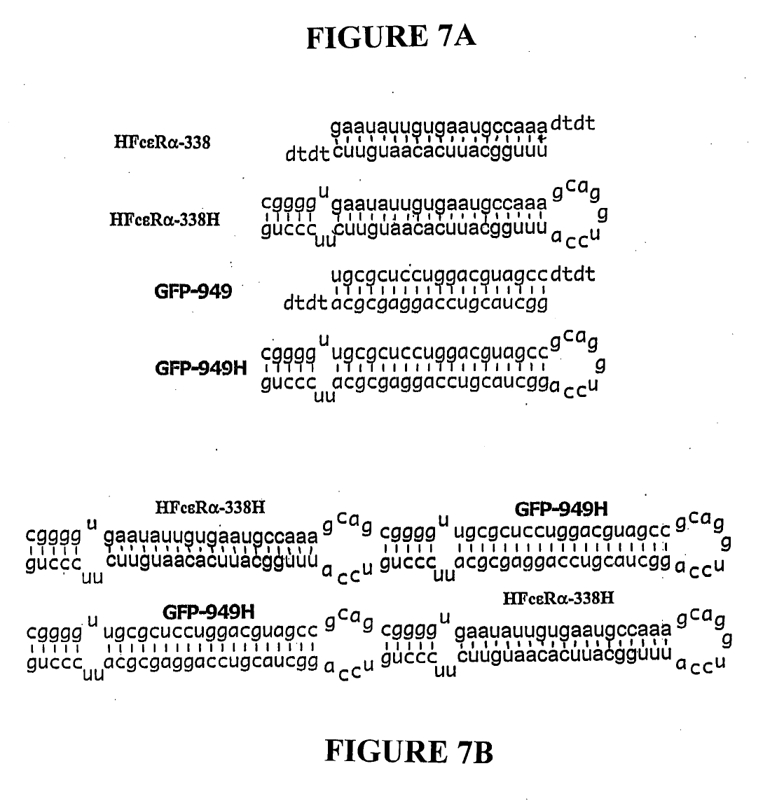

In another embodiment, an inventive RNAi agent (e.g., an shRNA) is generated as a single transcript, for example by transcription of a single transcription unit comprising self complementary regions. FIG. 6 depicts one such embodiment of the present invention. As indicated, a template is employed that includes first and second complementary regions, and optionally includes a loop region. Such a template may be utilized for in vitro or in vivo transcription, with appropriate selection of promoter (and optionally other regulatory elements). The present invention encompasses gene constructs capable of serving as templates for transcription of one or more siRNA or shRNA strands.

In vitro transcription may be performed using a variety of available systems including the T7, SP6, and T3 promoter/polymerase systems (e.g., those available commercially from Promega, Clontech, New England Biolabs, etc.). As will be appreciated by one of ordinary skill in the art, use of the T7 or T3 promoters typically requires an siRNA sequence having two G residues at the 5′ end while use of the SP6 promoter typically requires an siRNA sequence having a GA sequence at its 5′ end. Vectors including the T7, SP6, or T3 promoter are well known in the art and can readily be modified to direct transcription of siRNAs. When siRNAs are synthesized in vitro they may be allowed to hybridize before transfection or delivery to a subject. It is to be understood that inventive siRNA compositions need not consist entirely of double-stranded (hybridized) molecules. For example, siRNA compositions may include a small proportion of single-stranded RNA. This may occur, for example, as a result of the equilibrium between hybridized and unhybridized molecules, because of unequal ratios of sense and antisense RNA strands, because of transcriptional termination prior to synthesis of both portions of a self-complementary RNA, etc. Generally, preferred compositions comprise at least approximately 80% double-stranded RNA, at least approximately 90% double-stranded RNA, at least approximately 95% double-stranded RNA, or even at least approximately 99-100% double-stranded RNA. However, the siRNA compositions may contain less than 80% hybidized RNA provided that they contain sufficient double-stranded RNA to be effective.

Those of ordinary skill in the art will appreciate that, where inventive siRNA agents are to be generated in vivo, it is generally preferable that they be produced via transcription of one or more transcription units. The primary transcript may optionally be processed (e.g., by one or more cellular enzymes) in order to generate the final agent that accomplishes gene inhibition. It will further be appreciated that appropriate promoter and/or regulatory elements can readily be selected to allow expression of the relevant transcription units in mammalian cells. In some embodiments of the invention, it may be desirable to utilize a regulatable promoter; in other embodiments, constitutive expression may be desired.

In certain preferred embodiments of the invention, the promoter utilized to direct in vivo expression of one or more siRNA or shRNA transcription units is a promoter for RNA polymerase III (Pol III). Pol III directs synthesis of small transcripts that terminate within a stretch of 4-5 T residues. Certain Pol III promoters such as the U6 or H1 promoters do not require cis-acting regulatory elements (other than the first transcribed nucleotide) within the transcribed region and thus are preferred according to certain embodiments of the invention since they readily permit the selection of desired siRNA sequences. In the case of naturally occurring U6 promoters the first transcribed nucleotide is guanosine, while in the case of naturally occurring H1 promoters the first transcribed nucleotide is adenine. (See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Thus in certain embodiments of the invention, e.g., where transcription is driven by a U6 promoter, the 5′-nucleotide of preferred shRNA sequences is G. In certain other embodiments of the invention, e.g., where transcription is driven by an H1 promoter, the 5′ nucleotide may be A.

According to certain embodiments of the invention promoters for RNA polymerase II (Pol II) may also be used as described, for example, in Xia, H., et al., Nat. Biotechnol., 20, pp. 1006-1010, 2002. As described therein, constructs in which a hairpin sequence is juxtaposed within close proximity to a transcription start site and followed by a polyA cassette, resulting in minimal to no overhangs in the transcribed hairpin, may be employed. In certain embodiments of the invention tissue-specific, cell-specific, or inducible Pol II promoters may be used, provided the foregoing requirements are met. For example, it may be desirable to use mast cell specific, T cell specific, or B cell specific promoters. Certain of the target genes described herein comprise such promoters, and others are known in the art.

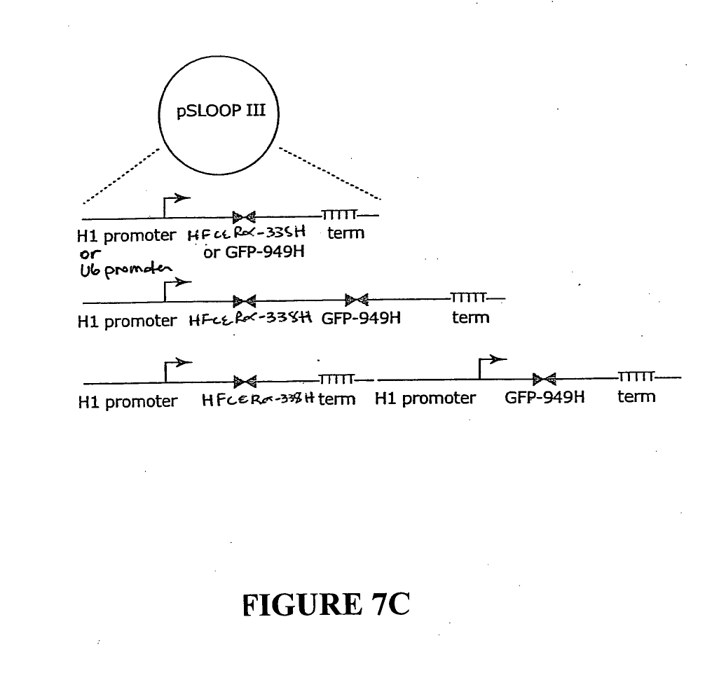

It will be appreciated that in vivo expression of constructs such as those depicted in FIGS. 7 and 8 can desirably be accomplished by introducing the constructs into a vector and introducing the vector into mammalian cells, e.g., in a subject. Any of a variety of vectors may be selected, though in certain embodiments it may be desirable to select a vector that can deliver the construct(s) to one or more cells in the respiratory passages.

Either viral or non-viral vectors (e.g., plasmids) can be used. The present invention encompasses vectors containing siRNA and/or shRNA transcription units, as well as cells containing such vectors or otherwise engineered to contain expressable transcription units capable of serving as templates for transcription of one or more siRNA or shRNA strands. In certain preferred embodiments of the invention, inventive vectors are plasmids or gene therapy vectors appropriate for the delivery of an siRNA or shRNA expressing construct to mammalian cells. Such vectors may be administered to a subject before or after exposure to a stimulus suspected of causing an exacerbation of asthmatic or allergic symptoms, to provide prophylaxis or treatment for these conditions. The RNAi vectors of the invention may be delivered in a composition comprising any of a variety of delivery agents as described further below.

The invention therefore provides a variety of viral and nonviral vectors whose presence within a cell results in transcription of one or more RNAs that self-hybridize or hybridize to each other to form an shRNA or siRNA that inhibits expression of at least one transcript encoding a protein among those mentioned above in the cell. In certain embodiments of the invention two separate, complementary siRNA strands are transcribed using a single vector containing two promoters, each of which directs transcription of a single siRNA strand, i.e., is operably linked to a template for the siRNA so that transcription occurs. The two promoters may be in the same orientation, in which case each is operably linked to a template for one of the siRNA strands. Alternately, the promoters may be in opposite orientation flanking a single template so that transcription from the promoters results in synthesis of two complementary RNA strands.

In other embodiments of the invention a vector containing a promoter that drives transcription of a single RNA molecule comprising two complementary regions (e.g., an shRNA) is employed. In certain embodiments of the invention a vector containing multiple promoters, each of which drives transcription of a single RNA molecule comprising two complementary regions is used. Alternately, multiple different shRNAs may be transcribed, either from a single promoter or from multiple promoters. A variety of configurations are possible. For example, a single promoter may direct synthesis of a single RNA transcript containing multiple self-complementary regions, each of which may hybridize to generate a plurality of stem-loop structures. These structures may be cleaved in vivo, e.g., by DICER, to generate multiple different shRNAs. It will be appreciated that such transcripts preferably contain a termination signal at the 3′ end of the transcript but not between the individual shRNA units. It will also be appreciated that single RNAs from which multiple siRNAs can be generated need not be produced in vivo but may instead be chemically synthesized or produced using in vitro transcription and provided exogenously.

In another embodiment of the invention, the vector includes multiple promoters, each of which directs synthesis of a self-complementary RNA molecule that hybridizes to form an shRNA. The multiple shRNAs may all target the same transcript, or they may target different transcripts. Any combination of transcripts may be targeted. See, e.g., FIG. 7B. In general, according to certain embodiments of the invention the siRNAs and/or shRNAs expressed in the cell comprise a base-paired (duplex) region 15-29 nucleotides in length, e.g., approximately 19 nucleotides long.

Those of ordinary skill in the art will further appreciate that in vivo expression of siRNAs or shRNAs according to the present invention may allow the production of cells that produce the siRNA or shRNA over long periods of time (e.g., greater than a few days, preferably at least several weeks to months, more preferably at least a year or longer, possibly a lifetime).

Preferred viral vectors for use in the compositions to provide intracellular expression of siRNAs and shRNAs include, for example, retroviral vectors and lentiviral vectors. See, e.g., Kobinger, G. P., et al., Nat Biotechnol 19(3):225-30, 2001, describing a vector based on a Filovirus envelope protein-pseudotyped HIV vector, which efficiently transduces intact airway epithelium from the apical surface. See also Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

In certain embodiments of the invention the vector is a lentiviral vector whose presence within a cell results in transcription of one or more RNAs that self-hybridize or hybridize to each other to form an shRNA or siRNA that inhibits expression of at least one transcript in the cell. For purposes of description it will be assumed that the vector is a lentiviral vector. Suitable lentiviral vectors are described, for example, in Rubinson, D., et al, Nature Genetics, Vol. 33, pp. 401-406, 2003. However, it is to be understood that other retroviral or lentiviral vectors may also be used. According to various embodiments of the invention the lentiviral vector may be either a lentiviral transfer plasmid or a lentiviral particle, e.g., a lentivirus capable of infecting cells. In certain embodiments of the invention the lentiviral vector comprises a nucleic acid segment operably linked to a promoter, so that transcription results in synthesis of an RNA comprising complementary regions that hybridize to form an shRNA targeted to the target transcript. According to certain embodiments of the invention the shRNA comprises a base-paired region approximately 19 nucleotides long. According to certain embodiments of the invention the RNA may comprise more than 2 complementary regions, so that self-hybridization results in multiple base-paired regions, separated by loops or single-stranded regions. The base-paired regions may have identical or different sequences and thus may be targeted to the same or different regions of a single transcript or to different transcripts.

In certain embodiments of the invention the lentiviral vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. According to certain embodiments of the invention the siRNA comprises a base-paired region approximately 19 nucleotides long. In certain embodiments of the invention the lentiviral vector comprises at least two promoters and at least two nucleic acid segments, wherein each promoter is operably linked to a nucleic acid segment, so that transcription from the promoters results in synthesis of two complementary RNAs that hybridize with each other to form an siRNA targeted to the target transcript.

As mentioned above, the lentiviral vectors may be lentiviral transfer plasmids or infectious lentiviral particles (e.g., a lentivirus or pseudotyped lentivirus). See, e.g., U.S. Pat. No. 6,013,516 and references 113-117 for further discussion of lentiviral transfer plasmids, lentiviral particles, and lentiviral expression systems. As is well known in the art, lentiviruses have an RNA genome. Therefore, where the lentiviral vector is a lentiviral particle, e.g., an infectious lentivirus, the viral genome must undergo reverse transcription and second strand synthesis to produce DNA comprising a template for RNA transcription. In addition, where reference is made herein to elements such as promoters, regulatory elements, etc., it is to be understood that the sequences of these elements are present in RNA form in the lentiviral particles of the invention and are present in DNA form in the lentiviral transfer plasmids of the invention. Furthermore, where a template for synthesis of an RNA is “provided by” RNA present in a lentiviral particle, it is understood that the RNA must undergo reverse transcription and second strand synthesis to produce DNA that can serve as a template for synthesis of RNA (transcription). Vectors that provide templates for synthesis of siRNA or shRNA are considered to provide the siRNA or shRNA when introduced into cells in which such synthesis occurs.

Inventive siRNAs or shRNAs may be introduced into cells by any available method. For instance, siRNAs, shRNAs, or vectors encoding them can be introduced into cells via conventional transformation or transfection techniques. As used herein, the terms “transformation” and “transfection” are intended to refer to a variety of art-recognized techniques for introducing foreign nucleic acid (e.g., DNA or RNA) into a cell, including calcium phosphate or calcium chloride co-precipitation, DEAE-dextran-mediated transfection, lipofection, etc. Injection or electroporation can also be used. As described below, one aspect of the invention includes the use of a variety of delivery agents for introducing siRNAs, shRNAs, and or vectors (either DNA vectors or viral vectors) that comprise a template for synthesis of an siRNA or shRNA into cells including, but not limited to, cationic polymers; various peptide molecular transporters including arginine-rich peptides, histidine-rich peptides, and cationic and neutral lipids; various non-cationic polymers; liposomes; carbohydrates; and surfactant materials. The invention also encompasses the use of delivery agents that have been modified in any of a variety of ways, e.g., by addition of a delivery-enhancing moiety to the delivery agent, as described further below.

The present invention encompasses any cell manipulated to contain an inventive RNAi agent. Preferably, the cell is a mammalian cell, particularly human. In some embodiments of the invention, the cells are non-human cells within a non-human organism. For example, the present invention encompasses transgenic non-human animals engineered to contain or express inventive RNAi agents. Such animals are useful for studying the function and/or activity of inventive RNAi agents, and/or of the mechanisms involved in IgE production, IgE-mediated hypersensitivity, mast cell or basophil degranulation, etc. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can serve as a template for and direct the expression of an RNAi agent in one or more cell types or tissues of the transgenic animal. According to certain embodiments of the invention the transgenic animal is of a variety used as an animal model (e.g., rodents, sheep, or non-humane primates) for testing potential therapeutics for IgE-mediated diseases and conditions such as allergic rhinitis and asthma.

### III. RNAi Agents to Inhibit or Inactivate Mast Cells and Methods of Use

(A) RNAi Agents Targeted to Transcripts Encoding FcεRI Subunits

Mast cells bind to IgE through the high affinity IgE receptor, FcεRI. (See, e.g., Goldsby, R., et al., Kuby Immunology, 4^{th} Ed., W.H. Freeman, 2000, and references therein, for discussion of mast cells, the molecular mechanism of 1g-E mediated mast cell degranulation, and the role of mast cells in IgE-mediated conditions.) FcεRI consists of α, β, and γ chains. Although the γ chain is expressed by T cells and other cell types, the α and β chains are primarily expressed by mast cells and basophils in both mice and humans.

In accordance with one embodiment of the invention, FcεRI expression is inhibited by delivery of an RNAi agent (e.g., an siRNA, shRNA, or RNAi vector) targeted to a transcript encoding the α chain or the β chain of FcεR1. Such inhibition effectively disarms mast cells since without this receptor the cells cannot bind IgE and therefore cannot be activated by allergen. In certain embodiments of the invention RNAi agents targeted to transcripts encoding the FcεRI α and β chains are delivered individually or in combination. The invention provides RNAi agents, e.g., shRNAs, shRNAs, and RNAi vectors (e.g., plasmids, virus vectors, gene therapy vectors), targeted to transcripts encoding the FcεRI α or β chains and compositions (e.g., pharmaceutical compositions) comprising one or more of the inventive RNAi agents.

The invention provides a method of inhibiting expression of the FcεRI α chain comprising: administering to a cell or organism an RNAi agent targeted to a transcript encoding the FcεRI α chain. In particular, the invention provides a method of inhibiting expression of the FcεRI α chain comprising (i) administering to a cell or organism an siRNA or shRNA targeted to a transcript encoding the FcεRI α chain or (ii) administering to a cell or organism a nucleic acid that comprises a template for transcription of one or more RNA molecules that hybridize or self-hybridize to form an siRNA or shRNA targeted to a transcript encoding the FcεRI α chain.

The invention further provides a method of inhibiting expression of the FcεRI β chain comprising administering to a cell or organism an RNAi agent targeted to a transcript encoding the FcεRI β chain. In particular, the invention provides a method of inhibiting expression of the FcεRI β chain comprising (i) administering to a cell or organism an siRNA or shRNA targeted to a transcript encoding the FcεRI β chain or (ii) administering to a cell or organism a nucleic acid that comprises a template for transcription of one or more RNA molecules that hybridize or self-hybridize to form an siRNA or shRNA targeted to a transcript encoding the FcεRI β chain. The methods are useful for the prevention and treatment of diseases or conditions characterized by IgE-mediated hypersensitivity.

Sequences of some suitable target portions of the transcripts that encode the FcεRI α and β chains are listed in Tables 1 (α chain) and 2 (β chain). The sense strand of certain preferred inventive siRNAs comprises a portion having a sequence listed in Table 1 or Table 2. Certain preferred siRNAs comprise an antisense strand comprising a portion that is 100% complementary to a target portion listed in Table 1 or Table 2. shRNAs having a first portion whose sequence comprises a portion that is 100% complementary to a sequence listed in Table 1 or 2 and a second portion whose sequence comprises the stem-forming complement of that sequence (separated from the first portion by an unrelated sequence that forms a loop) may readily be designed as described elsewhere herein.

(B) RNAi Agents Targeted to Transcripts Encoding c-Kit

Mast cell development and survival requires the expression of the cell surface receptor c-Kit. (See, e.g., Ashman, L. K., Int J Biochem Cell Biol. 1999 October; 31(10):1037-51, for a discussion of c-Kit and its role in the hematopoietic system). In accordance with one embodiment of the invention, c-Kit expression is inhibited by delivery of siRNAs targeted to transcripts encoding c-Kit. The invention provides RNAi agents, e.g., shRNAs, shRNAs, and RNAi vectors (e.g., plasmids, virus vectors, gene therapy vectors), targeted to a transcript that encodes c-Kit and compositions (e.g., pharmaceutical compositions) comprising one or more of the inventive RNAi agents.

The invention further provides methods of inhibiting expression of c-Kit comprising administering to a cell or organism an RNAi agent targeted to a transcript that encodes c-Kit. In particular, the invention provides a method of inhibiting expression of c-Kit by administering to a cell or organism an siRNA or shRNA targeted to a transcript encoding c-Kit or (ii) administering to a cell or organism a nucleic acid that comprises a template for transcription of one or more RNA molecules that hybridize or self-hybridize to form an siRNA or shRNA targeted to a transcript encoding c-Kit. The methods are useful for the prevention and treatment of diseases or conditions characterized by IgE-mediated hypersensitivity.

Sequences of some suitable target portions of the transcript that encode c-Kit are listed in Table 3. The sense strand of certain preferred inventive siRNAs comprises a portion having a sequence listed in Table 3. Certain preferred siRNAs comprise an antisense strand comprising a portion that is 100% complementary to a target portion listed in Table 3. shRNAs having a first portion whose sequence comprises a portion that is 100% complementary to a sequence listed in Table 3 and a second portion whose sequence comprises the stem-forming complement of that sequence (separated from the first portion by an unrelated sequence that forms a loop) may readily be designed as described elsewhere herein.

(C) RNAi Agents Targeted to Transcripts Encoding Lyn or Syk

As mentioned above, crosslinking of FcεRI on mast cells activates intracellular signaling pathways, which ultimately results in degranulation. Two of the important signaling molecules are the protein tyrosine kinases Lyn and Syk. Studies have shown that mice deficient in Lyn and Syk are deficient in mast cell function. (See, e.g., Costello, P, et al., Oncogene, 13(12): 2595, 1996; Zhang, S., et al., Mol. Cell. Biol., 22(23): 8144-8154, 2002.) The inventors have recognized that inhibiting expression of Lyn or Syk at the level of transcription or translation using RNAi is a powerful method for controlling mast cell function and thus reducing the role of mast cells in disease. In accordance with one embodiment of the invention, Lyn and/or Syk expression is inhibited by delivery of one or more RNAi agents targeted to transcripts encoding these proteins.

The invention provides RNAi agents, such as siRNAs, shRNAs, or RNAi vectors targeted to Lyn or Syk transcripts, compositions (e.g., pharmaceutical compositions) comprising the inventive RNAi agents and vectors (including plasmids, virus vectors, and gene therapy vectors) for producing inventive siRNAs or shRNAs either as individual sense and antisense RNA strands (siRNAs) or as a single, self-complementary siRNA strand (shRNAs).

The invention further provides methods of inhibiting expression of Lyn comprising administering to a cell or organism an RNAi agent targeted to a transcript that encodes Lyn. In particular, the invention provides a method of inhibiting expression of Lyn comprising administering to a cell or organism an siRNA or shRNA targeted to a transcript encoding Lyn to a cell or organism or (ii) administering to a cell or organism a nucleic acid that comprises a template transcription of one or more RNA molecules that hybridize or self-hybridize to form an siRNA or shRNA targeted to a transcript encoding Lyn. The methods are useful for the prevention and treatment of diseases or conditions characterized by IgE-mediated hypersensitivity.

Sequences of some suitable target portions of the transcript that encodes Lyn are listed in Table 4. The sense strand of certain preferred inventive siRNAs comprises a portion having a sequence listed in Table 4. Certain preferred siRNAs comprise an antisense strand comprising a portion that is 100% complementary to a target portion listed in Table 4. shRNAs having a first portion whose sequence comprises a portion that is 100% complementary to a sequence listed in Table 4 and a second portion whose sequence comprises the stem-forming complement of that sequence (separated from the first portion by an unrelated sequence that forms a loop) may readily be designed as described elsewhere herein.

The invention further provides methods of inhibiting expression of Syk comprising administering to a cell or organism an RNAi agent targeted to a transcript that encodes Syk. In particular, the invention provides a method of inhibiting expression of Syk comprising (i) administering to a cell or organism an siRNA or shRNA, targeted to a transcript encoding Syk or (ii) administering to a cell or organism a nucleic acid that comprises a template for transcription of one or more RNA molecules that hybridize or self-hybridize to form an siRNA or shRNA targeted to a transcript encoding Syk. The methods are useful for the prevention and treatment of diseases or conditions characterized by IgE-mediated hypersensitivity.

Sequences of some suitable target portions of the transcript that encodes Syk are listed in Table 5. The sense strand of certain preferred inventive siRNAs comprises a portion having a sequence listed in Table 5. Certain preferred siRNAs comprise an antisense strand comprising a portion that is 100% complementary to a target portion listed in Table 5. shRNAs having a first portion whose sequence comprises a portion that is 100% complementary to a sequence listed in Table 5 and a second portion whose sequence comprises the stem-forming complement of that sequence (separated from the first portion by an unrelated sequence that forms a loop) may readily be designed as described elsewhere herein.

### IV. RNAi Agents to Inhibit IgE Production and Methods of Use Thereof

As mentioned above, serum IgE plays a major role in mast cell-mediated allergic rhinitis and asthma. The B cell antibody response, leading to IgE secretion, generally requires T cell help. T cell activation typically involves presentation of allergen to T cells by antigen presenting cells (APC), which include dendritic cells (DC), macrophages, and B cells Immature dendritic cells normally reside underneath the epithelium in various sites within the body (e.g., skin and mucosae). When these cells encounter allergens, they engulf them and migrate to the draining lymph nodes, where they present peptide fragments derived from processed allergens to T cells. A co-stimulatory molecule, termed ICOS (which stands for “inducible costimulator”), on dendritic cells may play a major role in the induction of T cells that will promote B cells to produce IgE. (See, e.g., Tafuri, A, et al., Nature, 409:105-9, 2001; Gonzalo, J. A., et al., Nat. Immunol., 2(7):597-604, 2001.) Thus in the absence of ICOS expression allergen stimulation does not lead to the secretion of IgE by B cells. In accordance with one embodiment of the invention, ICOS expression is inhibited by delivery of RNAi agents, such as siRNAs or shRNAs or RNAi vectors, targeted to transcripts encoding ICOS.

Accordingly, the invention provides RNAi agents targeted to ICOS transcripts, compositions (e.g., pharmaceutical compositions) comprising the inventive RNAi agents, and vectors (e.g., plasmids, virus vectors, gene therapy vectors) for producing the siRNAs and/or shRNAs either as individual sense and antisense RNA strands (siRNAs) or as a single, self-complementary RNA molecule (shRNAs). The invention provides a method of inhibiting expression of ICOS comprising administering to a cell or organism an RNAi agent targeted to a transcript that encodes ICOS. In particular, the invention provides a method of inhibiting expression of ICOS comprising (i) administering to a cell or organism an RNAi agent, such as an siRNA or shRNA, targeted to a transcript encoding ICOS or (ii) administering to a cell or organism an RNAi vector comprising a nucleic acid that comprises a template for transcription of one or more RNA molecules that hybridize or self-hybridize to form an siRNA or shRNA targeted to a transcript encoding ICOS. The methods are useful for the prevention and treatment of diseases or conditions characterized by IgE-mediated hypersensitivity.

Sequences of some suitable target portions of transcripts that encode ICOS the are listed in Table 6. The sense strand of certain preferred inventive siRNAs comprises a portion having a sequence listed in Table 6. Certain preferred siRNAs comprise an antisense strand comprising a portion that is 100% complementary to a target portion listed in Table 6. shRNAs having a first portion whose sequence comprises a portion that is 100% complementary to a sequence listed in Table 6 and a second portion whose sequence comprises the stem-forming complement of that sequence (separated from the first portion by an unrelated sequence that forms a loop) may readily be designed as described elsewhere herein.

### V. RNAi Inhibition of Genes in Dendritic Cells and Macrophages

The B cell antibody response that leads to IgE secretion generally requires T cell help, typically from type 2 T helper cells (Th2). While not wishing to be bound by any theory, it is likely that allergen specific memory Th2 cells are involved in stimulating the B cell antibody response following their own activation. Activation of Th2 cells typically requires presentation of allergen to these cells by APC. Among these, dendritic cells are particularly important. When DC encounter antigens they engulf them and migrate to draining lymph nodes, where they present peptide fragments from allergens to T cells. Depending on the activation status and expression of various genes, dendritic cells can either activate or inactivate the T cells that recognize the DC-presented allergens (as peptide-MHC complexes). See Banchereau, J. and Steinman, R., Nature, 392: 245-252, 1998, for a review of DCs and their functions.

The present invention encompasses the recognition that altering the interaction between T cells and antigen presenting cells (e.g., dendritic cells and macrophages) using RNAi provides an approach to inactivating, and/or completely or partly eliminang T cells that are involved in the allergic rhinitis and asthma, thereby achieving a therapeutic effect.

The invention provides RNAi agents, e.g., siRNAs, shRNAs, and RNAi vectors targeted to transcripts encoding a variety of proteins that play a role in antigen presentation and T cell activation by APC. These proteins and RNAi agents are described in further detail below.

(A) RNAi Agents Targeted to Transcripts Encoding FcεRI α Chain

As mentioned above, FcεRI, consisting of α, β, and γ chains, is the high affinity receptor for IgE. In addition to mast cells, DC from patients with atopic rhinitis and asthma also express the receptor, in contrast to the typical situation in individuals not suffering from these conditions. (See, e.g., Novak, N., et al., J. Clin. Invest., 111(7):1047, 2003, and references therein.) The FcεRI receptor expressed by DC is a trimeric form lacking the β chain. While not wishing to be bound by any theory, FcεRI on DC may function as an antigen-focusing molecule for efficient uptake, processing, and presentation to T cells. Because the α chain binds IgE, in accordance with the invention RNAi agents targeted to transcripts encoding the α chain are used to inhibit FcεRI expression by DC and/or macrophages. RNAi agents targeted to transcripts that encode FcεR1 are discussed above.

(B) RNAi Agents Targeted to Transcripts Encoding OX40 Ligand (OX40L)

OX40 and OX40L are a receptor-ligand pair important for T cell co-stimulation, i.e., stimulation of T cells by other cells (Gramaglia, I., et al., J Immunol., 161(12):6510-7, 1998). OX40 is expressed in activated T cells, whereas OX40L is expressed in DC, B cells, microglial cells, and endothelial cells. In a mouse model of allergic asthma, OX40L has been strongly implicated in the Th2 response in allergic inflammation (Hoshino, A., et al., Eur. J. Immunol., 33(4):861-9, 2003). In accordance with the invention RNAi agents targeted to transcripts encoding OX40L are used to inhibit OX40L expression by DC and/or macrophage, thereby interfering with Th2 cell response to these APC. According to the invention, reduced Th2 response, resulting from inhibiting synthesis of OX40L, leads to reduced production of IgE, thus decreasing degranulation of mast cells and resulting in a therapeutic effect.

The invention provides RNAi agents, e.g., siRNAs, shRNAs, and RNAi vectors targeted to transcripts encoding OX40L, compositions (e.g., pharmaceutical compositions) comprising the inventive RNAi agents, and vectors (including plasmids and gene therapy vectors) for producing the RNAi agents either as individual sense and antisense RNA strands (siRNAs) or as a single, self-complementary RNA molecule (shRNAs). The invention provides a method of inhibiting expression of OX40L comprising administering an RNAi agent targeted to a transcript that encodes OX40L. In particular, the invention provides methods of inhibiting expression of OX40L comprising (i) administering to a cell or organism an RNAi agent, such as an siRNA or shRNA, targeted to a transcript encoding OX40L or (ii) administering to a cell or organism an RNAi vector comprising a nucleic acid that comprises a template for transcription of one or more RNA molecules that hybridize or self-hybridize to form an siRNA or shRNA targeted to a transcript encoding OX40L. The methods are useful for the prevention and treatment of diseases or conditions characterized by IgE-mediated hypersensitivity.

Sequences of some suitable target portions of transcripts that encode OX40L are listed in Table 7. The sense strand of certain preferred inventive siRNAs comprises a portion having a sequence listed in Table 7. Certain preferred siRNAs comprise an antisense strand comprising a portion that is 100% complementary to a target portion listed in Table 7. shRNAs having a first portion whose sequence comprises a portion that is 100% complementary to a sequence listed in Table land a second portion whose sequence comprises the stem-forming complement of that sequence (separated from the first portion by an unrelated sequence that forms a loop) may readily be designed as described elsewhere herein.

(C) RNAi Agents Targeted to CD40

CD40 expression is induced upon activation of APC. Interaction of CD40 with CD40L (CD 154) on activated T cells plays a highly important role in the T cell response. (See Curr Opin Hematol. 2003 July; 10(4):272-8, 2003 for a review on CD40 and its role immunity and tolerance). The lack of CD40 expression by APC is sufficient for induction of tolerance (e.g., lack of or significant decrease in the response to antigen in a subject relative to a usual or previous response). In accordance with the invention one or more RNAi agents targeted to CD40 transcripts are used to inhibit CD40 expression by DC and/or macrophages, thereby interfering with Th2 cell response to these APC. Reduced Th2 response leads to reduced production of IgE, thus decreasing degranulation of mast cells and resulting in a therapeutic effect.

The invention provides RNAi agents, including siRNAs, shRNAs, and RNAi vectors targeted to transcripts encoding CD40, pharmaceutical compositions comprising the inventive siRNAs, shRNAs, and vectors (including plasmids, virus vectors, and gene therapy vectors) for producing the siRNAs as individual sense and antisense RNA strands or producing shRNAs as a single, self-complementary RNA molecule. The invention provides a method of inhibiting expression of CD40 comprising administering to a cell or organism an RNAi agent targeted to a transcript that encodes CD40. In particular, the invention provides methods of inhibiting expression of CD40 comprising (i) administering to a cell or organism an siRNA or shRNA targeted to a transcript encoding CD40 or (ii) administering to a cell or organism a nucleic acid that comprises a template for transcription of one or more RNA molecules that hybridize or self-hybridize to form an siRNA or shRNA targeted to a CD40 transcript. The methods are useful for the prevention and treatment of diseases or conditions characterized by IgE-mediated hypersensitivity.

Sequences of some suitable target portions of the genes that encode CD40 are listed in Table 8. The sense strand of certain preferred inventive siRNAs comprises a portion having a sequence listed in Table 8. Certain preferred siRNAs comprise an antisense strand comprising a portion that is 100% complementary to a target portion listed in Table 8. shRNAs having a first portion whose sequence comprises a portion that is 100% complementary to a sequence listed in Table 8 and a second portion whose sequence comprises the stem-forming complement of that sequence (separated from the first portion by an unrelated sequence that forms a loop) may readily be designed as described elsewhere herein.

(D) RNAi Agents Targeted to CD80 and CD86

CD80 and CD86 are co-stimulatory molecules that play an extremely important role in T cell activation. They interact with CD28 and CTLA4 expressed on T cells. In accordance with the invention RNAi agents targeted to transcripts that encode CD80 or CD86 are used to inhibit expression of CD80 and/or CD86 by DC and/or macrophage, thereby interfering with Th2 cell responses to these APC. Reduced Th2 response leads to reduced production of IgE, thus decreasing degranulation of mast cells and resulting in a therapeutic effect.

The invention provides RNAi agents, e.g., siRNAs, shRNAs, and RNAi vectors targeted to transcripts encoding CD80 or CD86, compositions (e.g., pharmaceutical compositions) comprising the inventive RNAi agents, and vectors (including plasmids, virus vectors, gene therapy vectors) for producing RNAi agents either as individual sense and antisense RNA strands (siRNAs) or as a single, self-complementary RNA molecule (shRNAs). The invention further provides methods of inhibiting expression of CD80 comprising administering to a cell or organism and RNAi agent targeted to CD80. In particular, the invention provides a method of inhibiting expression of CD80 comprising (i) administering to a cell or organism an siRNA or shRNA targeted to a transcript encoding CD80 or (ii) administering to a cell or organism a nucleic acid that comprises a template for transcription of one or more RNA molecules that hybridize or self-hybridize to form an siRNA or shRNA targeted to a transcript encoding CD80. The methods are useful for the prevention and treatment of diseases or conditions characterized by IgE-mediated hypersensitivity. The invention additionally provides methods of inhibiting expression of CD86 comprising administering to a cell or organism an RNAi agent targeted to CD86. In particular, the invention provides a method of inhibiting expression of CD86 comprising (i) administering to a cell or organism an siRNA or shRNA targeted to a transcript encoding CD86 or (ii) administering to a cell or organism a nucleic acid that comprises a template for transcription of one or more RNA molecules that hybridize or self-hybridize to form an siRNA or shRNA targeted to a transcript encoding CD86. The methods are useful for the prevention and treatment of diseases or conditions characterized by IgE-mediated hypersensitivity. According to certain embodiments of the invention RNAi agents targeted to CD80 and CD86 are administered either individually or in combination.

Sequences of some suitable target portions of transcripts that encode CD80 or CD86 are listed in Tables 9 and 10. The sense strand of certain preferred inventive siRNAs comprises a portion having a sequence listed in Table 9 or Table 10. Certain preferred siRNAs comprise an antisense strand comprising a portion that is 100% complementary to a target portion listed in Table 9 or Table 10. shRNAs having a first portion whose sequence comprises a portion that is 100% complementary to a sequence listed in Table 9 or 10 and a second portion whose sequence comprises the stem-forming complement of that sequence (separated from the first portion by an unrelated sequence that forms a loop) may readily be designed as described elsewhere herein.

(E) RNAi Agents Targeted to Re1 Family Members

The NF-κB family of transcription factors is induced in activated DC and macrophages (Granelli-Piperno, A., et al., Proc. Natl. Acad. Sci. USA, 92(24): 10944, 1995).

NF-κB consists of five family members: p50, p52, Re1A (p65), c-Re1, and Re1B. Re1B/p50 heterodimer is associated with increased APC function and up-regulation of CD40. Deficiency of Re1B in DC may suppress the autoimmune response. (Valero, R. et al., J. Immunol., 169(1):185-92, 2002.) In accordance with the invention siRNAs targeted to transcripts encoding Re1B are used to inhibit Re1B expression by DC and/or macrophage, thereby interfering with Th2 cell response to these APC. Reduced Th2 response leads to reduced production of IgE, thus decreasing degranulation of mast cells and resulting in a therapeutic effect.

The invention provides RNAi agents, including siRNAs, shRNAs, and RNAi vectors targeted to transcripts encoding Re1A or Re1B, compositions, e.g., pharmaceutical compositions, comprising the inventive RNAi agents, and vectors (including plasmids, virus vectors, and gene therapy vectors) for producing them either as individual sense and antisense RNA strands (siRNAs) or as a single, self-complementary RNA molecule (shRNAs). The invention additionally provides methods of inhibiting expression of Re1B comprising (i) administering to a cell or organism an RNAi agent (e.g., an siRNA or shRNA) targeted to a transcript encoding Re1A or Re1B or (ii) administering to a cell or organism a nucleic acid that comprises a template for transcription of one or more RNA molecules that hybridize or self-hybridize to form an siRNA or shRNA targeted to a transcript encoding Re1A or Re1B. The methods are useful for the prevention and treatment of diseases or conditions characterized by IgE-mediated hypersensitivity.

Sequences of some suitable target portions of the genes that encode Re1A or Re1B are listed in Tables 11 and 12. The sense strand of certain preferred inventive siRNAs comprises a portion having a sequence listed in Table 11 or Table 12. Certain preferred siRNAs comprise an antisense strand comprising a portion that is 100% complementary to a target portion listed in Table 11 or Table 12. shRNAs having a first portion whose sequence comprises a portion that is 100% complementary to a sequence listed in Table 11 or 12 and a second portion whose sequence comprises the stem-forming complement of that sequence (separated from the first portion by an unrelated sequence that forms a loop) may readily be designed as described elsewhere herein.

(F) RNAi Agents Targeted to 4-1 BB Ligand

4-1BB and 4-1BB ligand are another receptor-ligand pair for T cell co-stimulation. 4-1BB is expressed in activated T cells, whereas 4-1BB ligand is expressed in DC upon encounter with pathogens. (See, Croft, M., Cytokine Growth Factor Rev. 2003 Jun.-Aug.; 14(3-4):265-73, 2003 and references therein for a review of the role of 4-1BB and other co-stimulatory molecules. See also Kwon, B., et al., Trends Immunol., 23(8):378-80, 2002.) In accordance with the invention RNAi agents targeted to transcripts encoding 4-1 BB ligand are used to inhibit 4-1 BB ligand expression by DC and/or macrophage, thereby interfering with Th2 cell response to these APC. Reduced Th2 response leads to reduced production of IgE, thus decreasing degranulation of mast cells and resulting in a therapeutic effect.

The invention provides RNAi agents, such as siRNAs, shRNAs, and RNAi vectors targeted to transcripts encoding 4-1 BB ligand, compositions, e.g., pharmaceutical compositions comprising the inventive RNAi agents, and vectors (including plasmids, virus vectors, gene therapy vectors) for producing them either as individual sense and antisense RNA strands (siRNAs) or as a single, self-complementary RNA molecule (shRNAs). The invention additionally provides methods of inhibiting expression of 4-1BB ligand comprising (i) administering to a cell or organism an RNAi agent (e.g., an siRNA or shRNA) targeted to a transcript encoding 4-1BB ligand or (ii) administering to a cell or organism a nucleic acid that comprises a template for transcription of one or more RNA molecules that hybridize or self-hybridize to form an siRNA or shRNA targeted to a transcript encoding 4-1BB ligand. The methods are useful for the prevention and treatment of diseases or conditions characterized by IgE-mediated hypersensitivity.

Sequences of some suitable target portions of the genes that encode the 4-1BB ligand are listed in Table 13. The sense strand of certain preferred inventive siRNAs comprises a portion having a sequence listed in Table 13 Certain preferred siRNAs comprise an antisense strand comprising a portion that is 100% complementary to a target portion listed in Table 13. shRNAs having a first portion whose sequence comprises a portion that is 100% complementary to a sequence listed in Table 13 and a second portion whose sequence comprises the stem-forming complement of that sequence (separated from the first portion by an unrelated sequence that forms a loop) may readily be designed as described elsewhere herein.

(G) RNAi Agents Targeted to Toll-like Receptors

Toll-like receptors are pattern recognition receptors that play a role in initiation of the immune response. See Dabbagh K, and Lewis D B., Curr Opin Infect Dis., 16(3):199-204, 2003, and Lien, E., Ann Allergy Asthma Immunol., 88(6):543-7, 2002, and references therein, for reviews. In accordance with the invention RNAi agents targeted to transcripts encoding Toll-like receptors are used to inhibit expression of these receptors by DC and/or macrophage, thereby interfering with Th2 cell response to these APC. Reduced Th2 response leads to reduced production of IgE, thus decreasing degranulation of mast cells and resulting in a therapeutic effect.

The invention provides RNAi agents, such as siRNAs and shRNAs, targeted to transcripts encoding Toll-like receptors, compositions, e.g., pharmaceutical compositions, comprising the RNAi agents, and vectors (including plasmids, virus vectors, gene therapy vectors) for producing them either as individual sense and antisense RNA strands (siRNAs) or as a single, self-complementary RNA molecule (shRNAs). The invention additionally provides methods of inhibiting expression of a Toll-like receptor comprising (i) administering to a cell or organism an RNAi agent such as an siRNA or shRNA targeted to a transcript encoding a Toll-like receptor or (ii) administering to a cell or organism a nucleic acid that comprises a template for transcription of one or more RNA molecules that hybridize or self-hybridize to form an siRNA or shRNA targeted to a transcript encoding a Toll-like receptor. The methods are useful for the prevention and treatment of diseases or conditions characterized by IgE-mediated hypersensitivity.

Sequences of some suitable target portions of the genes that encode the FcεRI α and β chains are listed in Tables 14-22. The sense strand of certain preferred inventive siRNAs comprises a portion having a sequence listed in Tables 14-22. Certain preferred siRNAs comprise an antisense strand comprising a portion that is 100% complementary to a target portion listed in Table 14-22. shRNAs having a first portion whose sequence comprises a portion that is 100% complementary to a sequence listed in Tables 14-22 and a second portion whose sequence comprises the stem-forming complement of that sequence (separated from the first portion by an unrelated sequence that forms a loop) may readily be designed as described elsewhere herein.

The invention also encompasses the use of the inventive RNAi agents for treatment of various other conditions in which activity of pathways involving activation of Toll-like receptors occurs. Such conditions include sepsis, shock, and burn-related injuries.

It has been shown that mice expressing either a mutant form of or no Toll-like receptor 4

(TLR4), a critical element of the mammalian endotoxin receptor, were resistant to postburn myocardial contractile dysfunction (Thomas J A, et al., Am J Physiol Heart Circ Physiol., 283(4):H1645-55, 2002). See Cristofaro P and Opal S M, Expert Opin Ther Targets, 7(5):603-12, 2003, and references therein for a review that discusses the role of Toll-like receptors in septic shock. The invention provides a method of treating sepsis, shock, or a burn-related injury comprising steps of: (i) providing a subject in need of treatment for sepsis, shock, or a burn-related injury; and (ii) administering to the subject a composition comprising an RNAi agent targeted to a Toll-like receptor. In certain embodiments of the invention the Toll-like receptor is TLR4. In certain embodiments of the invention the burn-related injury is myocardial injury, e.g., ischemia/reperfusion injury, cardiac myocyte apoptosis, etc. The inventive RNAi agents may be delivered using any of the methods described herein and/or using a catheter, e.g., for direct delivery to the heart.

(H) RNAi Agents Targeted to CD83

CD83 is strongly up-regulated with co-stimulatory molecules such as CD80 and CD86 during DC maturation. See Lechmann M., et al., Trends Immunol. 23(6):273-5, 2002 for a review of CD80 and its functions. DC-mediated T cell proliferation is completely inhibited by a soluble CD83 (Lechmann M., et al., J Exp Med., 194(12):1813-21, 2001). In accordance with the invention RNAi agents targeted to transcripts encoding CD83 are used to inhibit expression of CD83 by DC, thereby interfering with Th2 cell response to these APC. Reduced Th2 response leads to reduced production of IgE, thus decreasing degranulation of mast cells and resulting in a therapeutic effect.

The invention provides RNAi agents, such as siRNAs, shRNAs, and RNAi vectors targeted to transcripts encoding CD83, compositions, e.g., pharmaceutical compositions, comprising the inventive RNAi agents, and vectors (including plasmids, virus vectors, gene therapy vectors) for producing them either as individual sense and antisense RNA strands (siRNAs) or as a single, self-complementary RNA molecule (shRNAs). The invention additionally provides methods of inhibiting expression of CD83 comprising (i) administering to a cell or organism an RNAi agent such as an siRNA or shRNA targeted to a transcript encoding CD83 or (ii) administering to a cell or organism a nucleic acid that comprises a template for transcription of one or more RNA molecules that hybridize or self-hybridize to form an siRNA or shRNA targeted to a transcript encoding CD83. In certain embodiments of the invention an RNAi agent targeted to transcripts encoding CD83 is delivered together with an RNAi agent targeted to transcripts encoding CD80 and/or CD86.

Sequences of some suitable target portions of transcripts that encode CD83 are listed in Table 23. The sense strand of certain preferred inventive siRNAs comprises a portion having a sequence listed in Table 23. Certain preferred siRNAs comprise an antisense strand comprising a portion that is 100% complementary to a target portion listed in Table 23. shRNAs having a first portion whose sequence comprises a portion that is 100% complementary to a sequence listed in Table 23 and a second portion whose sequence comprises the stem-forming complement of that sequence (separated from the first portion by an unrelated sequence that forms a loop) may readily be designed as described elsewhere herein.

(I) RNAi Agents Targeted to SLAM

Signaling lymphocyte activation molecule (SLAM) is expressed on activated DC and directly augments production of inflammatory cytokines by T cells. See Veillette A. and Latour S., Curr Opin Immunol., 15(3):277-85, 2003, for a review of SLAM and its role in the immune system. In accordance with the invention siRNAs targeted to transcripts encoding SLAM are used to inhibit expression of SLAM by DC and/or macrophages, thereby interfering with Th2 cell response to these APC. Reduced Th2 response leads to reduced production of IgE, thus decreasing degranulation of mast cells and resulting in a therapeutic effect.

The invention provides RNAi agents, including siRNAs, shRNAs, and RNAi vectors targeted to transcripts encoding SLAM, compositions, e.g., pharmaceutical compositions, comprising the inventive RNAi agents, and vectors (including plasmids, virus vectors, and gene therapy vectors) for producing them either as individual sense and antisense RNA strands (siRNAs) or as a single, self-complementary siRNA strand (shRNAs). The invention additionally provides methods of inhibiting expression of SLAM comprising (i) administering to a cell or organism an RNAi agent such as an siRNA or shRNA targeted to a transcript encoding SLAM or (ii) administering to a cell or organism a nucleic acid that comprises a template for transcription of one or more RNA molecules that hybridize or self-hybridize to form an siRNA or shRNA targeted to a transcript encoding SLAM.

Sequences of some suitable target portions of transcripts that encode SLAM are listed in Table 24. The sense strand of certain preferred inventive siRNAs comprises a portion having a sequence listed in Table 24. Certain preferred siRNAs comprise an antisense strand comprising a portion that is 100% complementary to a target portion listed in Table 24. shRNAs having a first portion whose sequence comprises a portion that is 100% complementary to a sequence listed in Table 24 and a second portion whose sequence comprises the stem-forming complement of that sequence (separated from the first portion by an unrelated sequence that forms a loop) may readily be designed as described elsewhere herein.

(J) RNAi Agents Targeted to Common γ Chain

Activation of DC results in induction of IL-2R, IL-4R, IL-7R, and IL-15R. Expression of these receptors may be important for DC survival and function. These receptors all comprise a common γ chain. In accordance with the invention, inhibition of expression of these receptors simultaneously is achieved by delivery of siRNAs targeted to the common γ chain (γc), thereby interfering with DC survival and function. Reduced Th2 response leads to reduced production of IgE, thus decreasing degranulation of mast cells and resulting in a therapeutic effect.

The invention provides RNAi agents, including siRNAs, shRNAs, and RNAi vectors targeted to transcripts encoding the common γ chain, compositions, e.g., pharmaceutical compositions, comprising the inventive RNAi agents, and vectors (including plasmids, virus vectors, gene therapy vectors) for producing the RNAi agents either as individual sense and antisense RNA strands (siRNAs) or as a single, self-complementary RNA molecule (shRNAs). The invention additionally provides methods of inhibiting expression of the common γ chain comprising (i) administering to a cell or organism an RNAi agent such as an siRNA or shRNA targeted to a transcript encoding the common γ chain or (ii) administering to a cell or organism a nucleic acid that comprises a template for transcription of one or more RNA molecules that hybridize or self-hybridize to form an siRNA or shRNA targeted to a transcript encoding the common γ chain.

Sequences of some suitable target portions of the genes that encode the common γ chain are listed in Table 25. The sense strand of certain preferred inventive siRNAs comprises a portion having a sequence listed in Table 25. Certain preferred siRNAs comprise an antisense strand comprising a portion that is 100% complementary to a target portion listed in Table 25. shRNAs having a first portion whose sequence comprises a portion that is 100% complementary to a sequence listed in Table 25 and a second portion whose sequence comprises the stem-forming complement of that sequence (separated from the first portion by an unrelated sequence that forms a loop) may readily be designed as described elsewhere herein.

(K) RNAi Agents Targeted to Cyclooxygenase-2

Cyclooxygenase-2, also known as prostaglandin H synthase (PGHS), is the rate-limiting enzyme for the conversion of arachidonic acid to prostanoids. The induction and regulation of COX-2 may be key elements in the pathophysiological process of a number of inflammatory disorders and may play an important role in the pathogenesis of asthma. COX-2-deficient mice are thought to exhibit decreased allergic lung responses. COX-2 is induced in activated DC. In accordance with the invention an RNAi agent targeted to transcripts encoding COX-2 is used to inhibit expression of COX-2 by DC and/or macrophages, thereby interfering with Th2 cell response to these APC. Reduced Th2 response leads to reduced production of IgE, thus decreasing degranulation of mast cells and resulting in a therapeutic effect.

The invention provides RNAi agents, including siRNAs, shRNAs, and RNAi vectors targeted to transcripts encoding COX-2, compositions, e.g., pharmaceutical compositions, comprising the inventive RNAi agents, and vectors (including plasmids, virus vectors, and gene therapy vectors) for producing the RNAi agents either as individual sense and antisense RNA strands (siRNAs) or as a single, self-complementary RNA molecule (shRNAs). The invention additionally provides methods of inhibiting expression of the common γ chain comprising (i) administering to a cell or organism an RNAi agent such as an siRNA or shRNA targeted to a transcript encoding COX-2 or (ii) administering to a cell or organism a nucleic acid that comprises a template for transcription of one or more RNA molecules that hybridize or self-hybridize to form an siRNA or shRNA targeted to a transcript encoding COX-2.

Sequences of some suitable target portions of transcripts that encode COX-2 are listed in Table 26. The sense strand of certain preferred inventive siRNAs comprises a portion having a sequence listed in Table 26. Certain preferred siRNAs comprise an antisense strand comprising a portion that is 100% complementary to a target portion listed in Table 26. shRNAs having a first portion whose sequence comprises a portion that is 100% complementary to a sequence listed in Table 26 and a second portion whose sequence comprises the stem-forming complement of that sequence (separated from the first portion by an unrelated sequence that forms a loop) may readily be designed as described elsewhere herein.

### VI. Sequences

Tables 1-26 list sequences of preferred target portions of transcripts encoding

FCεR α chain, FCεR β chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2, respectively. Preferred siRNAs and shRNAs comprise an antisense strand comprising a portion that is substantially or 100% complementary to a sequence listed in Tables 1-26. The sequences of the sense strands of certain preferred siRNAs and shRNAs comprise a portion that is identical to a sequence listed in Tables 1-26. The sense strand sequences are listed in 5′ to 3′ direction according to the sequence present in the genome (genomic sequences contain T rather than U).

Each table contains sequences suitable for inhibiting expression of the human gene and sequences suitable for inhibiting expression of the corresponding mouse genes. In many cases the sequences are the same or very similar. The letter “H” preceding the name of the sequence indicates that it is targeted to the human gene, while the other sequences are targeted to the mouse gene. For example, FCεRα-268 denotes a sequence extending from position 268 to position 286 in the mouse mRNA that encodes FCεRα, including both positions 268 and 286. HFCεRα-338 denotes a sequence extending from position 338 to position 356 in the human gene, including both nucleotides 338 and 356. The tables include Genbank accession numbers of the human and mouse mRNAs.

TABLE 1
FcεRα Target Portions and RNAi
Agent Sense Strand Sequences

| Mouse | | |
|---|---|---|
| sequences | (Genbank accession number: NM_010184.1) | |
| FcεRα-268 | UUGGUCAUUGUGAGUGCCA | (SEQ ID NO: 1) |
| Fcεα-290 | CAAGACAGUGGAAAAUACA | (SEQ ID NO: 2) |
| FcεRα-310 | AUGUCAGAAGCAAGGAUUG | (SEQ ID NO: 3) |
| FcεRα-413 | UCCUUUGACAUCAGAUGCC | (SEQ ID NO: 4) |
| FcεRα-456 | GCAAGGUGAUCUACUACAG | (SEQ ID NO: 5) |
| FcεRα-673 | GAUUCUGUUUGCUGUGGAC | (SEQ ID NO: 6) |
| FcεRα-738 | GAGAUUCAGAAGACUGGAA | (SEQ ID NO: 7) |
| FcεRα-914 | CAGGAAUUGCAUAAAUGCU | (SEQ ID NO: 8) |
| Human | | |
| sequences | (Genbank accession number: NM_002001.1) | |
| HFcεRα-338 | GAAUAUUGUGAAUGCCAAA | (SEQ ID NO: 9) |
| HFcεRα-360 | GAAGACAGUGGAGAAUACA | (SEQ ID NO: 10) |
| HFcεRα-380 | AUGUCAGCACCAACAAGUU | (SEQ ID NO: 11) |
| HFcεRα-487 | UCUUCCUCAGGUGCCAUGG | (SEQ ID NO: 12) |
| HFcεRα-515 | CUGGGAUGUGUACAAGGUG | (SEQ ID NO: 13) |
| HFcεRα-743 | GAUUCUGUUUGCUGUGGAC | (SEQ ID NO: 14) |
| HFcεRα-818 | AACCAGGAAAGGCUUCAGA | (SEQ ID NO: 15) |
| HFcεRα-974 | CGUCUGUGCUCAAGGAUUU | (SEQ ID NO: 16) |

TABLE 2
FcεRβ Target Portions and RNAi
Agent Sense Strand Sequences

| Mouse | | |
|---|---|---|
| sequences | (Genbank accession number: J05019.1) | |
| FcεRβ-206 | GAUAUGCCUUUGUUUUGGA | (SEQ ID NO: 17) |
| FcεRβ-677 | UUACAGUGAGUUGGAAGAC | (SEQ ID NO: 18) |
| Human | | |
| sequences | (Genbank accession number: NM_000139.2) | |
| HFεcRβ-310 | GAUAUGCCUUUGUUUUGGA | (SEQ ID NO: 19) |
| HFεcRβ-784 | UUACAGUGAGUUGGAAGAC | (SEQ ID NO: 20) |

TABLE 3
c-Kit Target Portions and RNAi
Agent Sense Strand Sequences

| Mouse | | |
|---|---|---|
| sequences | (Genbank accession number: Y00864.1) | |
| c-Kit-356 | GUUUGUUAGAGAUCCUGCC | (SEQ ID NO: 21) |
| c-Kit-885 | GAUUCUGGAGUGUUCAUGU | (SEQ ID NO: 22) |
| c-Kit-924 | GGAUCAGCAAAUGUCACAA | (SEQ ID NO: 23) |
| c-Kit-1267 | CAAAACCAGAAAUCCUGAC | (SEQ ID NO: 24) |
| c-Kit-1517 | CAACGAUGUGGGCAAGAGU | (SEQ ID NO: 25) |
| c-Kit-1704 | CGAGGAGAUAAAUGGAAAC | (SEQ ID NO: 26) |
| c-Kit-1746 | CAACUUCCUUAUGAUCACA | (SEQ ID NO: 27) |
| c-Kit-1767 | UGGGAGUUUCCCAGAAACA | (SEQ ID NO: 28) |
| c-Kit-2020 | CCCUGGUCAUUACAGAAUA | (SEQ ID NO: 29) |
| c-Kit-2041 | GUUGCUAUGGUGAUCUUUU | (SEQ ID NO: 30) |
| c-Kit-2366 | UCCUCGCCUCCAAGAAUUG | (SEQ ID NO: 31) |
| c-Kit-2388 | UUCACAGAGACUUGGCAGC | (SEQ ID NO: 32) |
| c-Kit-2430 | CGGAUCACAAAGAUUUGCG | (SEQ ID NO: 33) |
| c-Kit-2457 | CUAGCCAGAGACAUCAGGA | (SEQ ID NO: 34) |
| c-Kit-2517 | GUGAAGUGGAUGGCACCAG | (SEQ ID NO: 35) |
| c-Kit-2574 | GUCUGGUCCUAUGGGAUUU | (SEQ ID NO: 36) |
| c-Kit-2669 | UCAAGGAAGGCUUCCGGAU | (SEQ ID NO: 37) |
| c-Kit-2727 | UCAUGAAGACUUGCUGGGA | (SEQ ID NO: 38) |
| c-Kit-4518 | UUCAGGUAUGUUGCCUUUA | (SEQ ID NO: 39) |
| c-Kit-5075 | ACUGUUGACAGUUCUGAAG | (SEQ ID NO: 40) |
| Human | | |
| sequences | (Genbank accession number: X06182.1) | |
| Hc-kit-346 | GUUUGUUAGAGAUCCUGCC | (SEQ ID NO: 41) |
| Hc-kit-812 | GGUGACUUCAAUUAUGAAC | (SEQ ID NO: 42) |
| He-kit-869 | GAUUCUGGAGUGUUCAUGU | (SEQ ID NO: 43) |
| Hc-kit-908 | GGAUCAGCAAAUGUCACAA | (SEQ ID NO: 44) |
| Hc-kit-1251 | CAAAACCAGAAAUCCUGAC | (SEQ ID NO: 45) |
| Hc-kit-1501 | UACAACGAUGUGGGCAAGA | (SEQ ID NO: 46) |
| Hc-kit-1700 | GAGGAGAUAAAUGGAAACA | (SEQ ID NO: 47) |
| Hc-kit-1742 | CAACUUCCUUAUGAUCACA | (SEQ ID NO: 48) |
| Hc-kit-1763 | AAUGGGAGUUUCCCAGAAA | (SEQ ID NO: 49) |
| Hc-kit-2016 | CCCUGGUCAUUACAGAAUA | (SEQ ID NO: 50) |
| Hc-kit-2037 | UUGUUGCUAUGGUGAUCUU | (SEQ ID NO: 51) |
| Hc-kit-2365 | UUCCUCGCCUCCAAGAAUU | (SEQ ID NO: 52) |
| Hc-kit-2387 | UUCACAGAGACUUGGCAGC | (SEQ ID NO: 53) |
| Hc-kit-2429 | CGGAUCACAAAGAUUUGUG | (SEQ ID NO: 54) |
| Hc-kit-2456 | CUAGCCAGAGACAUCAAGA | (SEQ ID NO: 55) |
| Hc-kit-2516 | UGUGAAGUGGAUGGCACCU | (SEQ ID NO: 56) |
| Hc-kit-2573 | GUCUGGUCCUAUGGGAUUU | (SEQ ID NO: 57) |
| Hc-kit-2668 | AUCAAGGAAGGCUUCCGGA | (SEQ ID NO: 58) |
| Hc-kit-2726 | UAAUGAAGACUUGCUGGGA | (SEQ ID NO: 59) |
| Hc-kit-4512 | GAUUCAGGUAUGUUGCCUU | (SEQ ID NO: 60) |
| Hc-kit-5061 | UGUUGACAGUUCUGAAGAA | (SEQ ID NO: 61) |

TABLE 4
Lyn Target Portions and RNAi
Agent Sense Strand Sequences

| Mouse | | |
|---|---|---|
| sequences | (Genbank accession number: M57696.1) | |
| Lyn-140 | GAAGACUCAACCAGUACGU | (SEQ ID NO: 62) |
| Lyn-172 | CUAUUUAUGUGAGAGAUCC | (SEQ ID NO: 63) |
| Lyn-196 | GUCCAAUAAACAGCAAAGG | (SEQ ID NO: 64) |
| Lyn-259 | AAGAUCCAGAGGAACAAGG | (SEQ ID NO: 65) |
| Lyn-626 | GCACUACAAAAUUAGAAGU | (SEQ ID NO: 66) |
| Lyn-777 | CAGAAGCCAUGGGAUAAAG | (SEQ ID NO: 67) |
| Lyn-864 | GUCUGGAUGGGUUACUAUA | (SEQ ID NO: 68) |
| Lyn-954 | GCCAACCUCAUGAAGACCU | (SEQ ID NO: 69) |
| Lyn-1058 | UAGUUUGCUGGAUUUCCUC | (SEQ ID NO: 70) |
| Lyn-1154 | UACAUCGAGCGGAAGAACU | (SEQ ID NO: 71) |
| Lyn-1271 | GUACACAGCAAGGGAAGGU | (SEQ ID NO: 72) |
| Lyn-1388 | GAUUGUCACCUAUGGGAAG | (SEQ ID NO: 73) |
| Lyn-1408 | UUCCCUACCCAGGGAGAAC | (SEQ ID NO: 74) |
| Lyn-1477 | UGGAGAACUGCCCAGAUGA | (SEQ ID NO: 75) |
| Human | | |
| sequences | (Genbank accession number: M16038.1) | |
| HLyn-352 | GAAGACUCAACCAGUACGU | (SEQ ID NO: 76) |
| HLyn-384 | CUAUUUAUGUGAGAGAUCC | (SEQ ID NO: 77) |
| HLyn-408 | UCCAAUAAACAGCAAAGGC | (SEQ ID NO: 78) |
| HLyn-470 | AAGAUCCAGAGGAACAAGG | (SEQ ID NO: 79) |
| HLyn-838 | GCACUACAAAAUUAGAAGU | (SEQ ID NO: 80) |
| HLyn-989 | CAGAAGCCAUGGGAUAAAG | (SEQ ID NO: 81) |
| HLyn-1076 | GUCUGGAUGGGUUACUAUA | (SEQ ID NO: 82) |
| HLyn-1166 | AAGCCAACCUCAUGAAGAC | (SEQ ID NO: 83) |
| HLyn-1270 | CAGUUUGCUGGAUUUCCUG | (SEQ ID NO: 84) |
| HLyn-1366 | UACAUCGAGCGGAAGAACU | (SEQ ID NO: 85) |
| HLyn-1483 | GUACACAGCAAGGGAAGGU | (SEQ ID NO: 86) |
| HLyn-1600 | AAUUGUCACCUAUGGGAAA | (SEQ ID NO: 87) |
| HLyn-1620 | AAUUCCCUACCCAGGGAGA | (SEQ ID NO: 88) |
| HLyn-1689 | UGGAGAACUGCCCAGAUGA | (SEQ ID NO: 89) |

TABLE 5
Syk Target Portions and RNAi
Agent Sense Strand Sequences

| Mouse | | |
|---|---|---|
| sequences | (Genbank accession number: U25685) | |
| Syk-172 | AGGAAGGCACACCACUACA | (SEQ ID NO: 90) |
| Syk-307 | AAGAAGCCCUUCAACCGGC | (SEQ ID NO: 91) |
| Syk-373 | AACCUCAUCAGGGAAUAUG | (SEQ ID NO: 92) |
| Syk-1009 | AUGGACACAGAGGUGUACG | (SEQ ID NO: 93) |
| Syk-1169 | UGAAAACCGUGGCUGUGAA | (SEQ ID NO: 94) |
| Syk-1450 | UUUGUGCACAGAGAUCUGG | (SEQ ID NO: 95) |
| Syk-1537 | CUGCGUGCUGAUGAAAACU | (SEQ ID NO: 96) |
| Syk-1606 | GAAUGCAUCAACUACUACA | (SEQ ID NO: 97) |
| Human | | |
| sequences | (Genbank accession number: L28824) | |
| HSyk-322 | AGGAAGGCACACCACUACA | (SEQ ID NO: 98) |
| HSyk-457 | AAGAAGCCCUUCAACCGGC | (SEQ ID NO: 99) |
| HSyk-523 | AACCUCAUCAGGGAAUAUG | (SEQ ID NO: 100) |
| HSyk-1174 | AUGGACACAGAGGUGUACG | (SEQ ID NO: 101) |
| HSyk-1334 | UGAAAACCGUGGCUGUGAA | (SEQ ID NO: 102) |
| HSyk-1615 | UUUGUGCACAGAGAUCUGG | (SEQ ID NO: 103) |
| HSyk-1702 | CUGCGUGCUGAUGAAAACU | (SEQ ID NO: 104) |
| HSyk-1771 | GAAUGCAUCAACUACUACA | (SEQ ID NO: 105) |

TABLE 6
ICOS Target Portions and RNAi
Agent Sense Strand Sequences

| Mouse | | |
|---|---|---|
| sequences | (Genbank accession number: NM_017480.1) | |
| ICOS-96 | UAACAGGAGAAAUCAAUGG | (SEQ ID NO: 107) |
| ICOS-578 | AGUGAAUACAUGUUCAUGG | (SEQ ID NO: 108) |
| ICOS-765 | AUUCUGCUGGUGUUUUGUU | (SEQ ID NO: 109) |
| ICOS-1665 | UAUUUAGCCUGAAAGCUGC | (SEQ ID NO: 110) |
| Human | | |
| sequences | (Genbank accession number: NM_012092.1) | |
| HICOS-76 | UAACAGGAGAAAUCAAUGG | (SEQ ID NO: 111) |
| HICOS-555 | GGUGAAUACAUGUUCAUGA | (SEQ ID NO: 112) |
| HICOS-735 | CUUCUGCUGGUGUUUUGUU | (SEQ ID NO: 113) |
| HICOS-1668 | CAUUUAGCCUGAAAGCUGC | (SEQ ID NO: 114) |

TABLE 7
OX40L Target Portions and RNAi
Agent Sense Strand Sequences

| Mouse | | |
|---|---|---|
| sequences | (Genbank accession number: U12763.1) | |
| OX40L-175 | GAUGAGAAUCUGGAAAACG | (SEQ ID NO: 115) |
| OX40L-211 | AAGUGGAAGAAGACGCUAA | (SEQ ID NO: 116) |
| OX40L-1279 | CUUCCUUCAAAGAACUACC | (SEQ ID NO: 117) |
| OX40L-1336 | UGCAAAGAAAACCAGGAGA | (SEQ ID NO: 118) |
| Human | | |
| sequences | (Genbank accession number: X79929.1) | |
| HOX40L-188 | AAGAUUCGAGAGGAACAAG | (SEQ ID NO: 119) |
| HOX40L-302 | GUAUCCUCGAAUUCAAAGU | (SEQ ID NO: 120) |
| HOX40L-668 | UGGUGAAUUCUGUGUCCUU | (SEQ ID NO: 121) |
| HOX40L-943 | UACUAGGCACCUUUGUGAG | (SEQ ID NO: 122) |

TABLE 8
CD40 Target Portions and RNAi
Agent Sense Strand Sequences

| Mouse | | |
|---|---|---|
| sequences | (Genbank accession number: M83312.1) | |
| CD40-299 | AGAAUCAGACACUGUCUGU | (SEQ ID NO: 123) |
| CD40-1242 | AACAGGUAGUGGAAUGAUG | (SEQ ID NO: 124) |
| CD40-1287 | AUUCCAAGGCAGGUAAGAU | (SEQ ID NO: 125) |
| CD40-1403 | UUGUCAUUUGACCUCCAUG | (SEQ ID NO: 126) |
| CD40-1422 | UGUGCUCUGUGGUAAUGUA | (SEQ ID NO: 127) |
| CD40-1452 | CACAUGUGCACAUAUCCUA | (SEQ ID NO: 128) |
| Human | | |
| sequences | (Genbank accession number: Z15017.1) | |
| HCD40-148 | GCUGUGUAUCUUCAUAGAA | (SEQ ID NO: 129) |
| HCD40-223 | ACGAUACAGAGAUGCAACA | (SEQ ID NO: 130) |
| HCD40-635 | UACUCAGAGCUGCAAAUAC | (SEQ ID NO: 131) |
| HCD40-707 | UUGAAUUGCAACCAGGUGC | (SEQ ID NO: 132) |
| HCD40-734 | UUGUCAAUGUGACUGAUCC | (SEQ ID NO: 133) |

TABLE 9
CD80 Target Portions and RNAi
Agent Sense Strand Sequences

| Mouse | | |
|---|---|---|
| sequences | (Genbank accession number: XM_148237.1) | |
| CD80-145 | CUACAUCUCUGUUUCUCGA | (SEQ ID NO: 134) |
| CD80-233 | CAAAGCAUCUGAAGCUAUG | (SEQ ID NO: 135) |
| CD80-1358 | CUUGAUGACUGAAGUGGAA | (SEQ ID NO: 136) |
| CD80-148 | GCAACUUGAUAUGUCAUGU | (SEQ ID NO: 137) |
| Human | | |
| sequences | (Genbank accession number: NM_005191.1) | |
| HCD80-257 | UCUUCUACGUGAGCAAUUG | (SEQ ID NO: 138) |
| HCD80-411 | CAAGUGUCCAUACCUCAAU | (SEQ ID NO: 139) |
| HCD80-472 | UCAGGUGUUAUCCACGUGA | (SEQ ID NO: 140) |
| HCD80-767 | UGGCUGAAGUGACGUUAUC | (SEQ ID NO: 141) |
| HCD80-1201 | AGGAAUGAGAGAUUGAGAA | (SEQ ID NO: 142) |
| HCD80-1271 | AAGAUCUGAAGGUAGCCUC | (SEQ ID NO: 143) |
| HCD80-1482 | AUGUUUCCAUUCUGCCAUC | (SEQ ID NO: 144) |

TABLE 10
CD86 Target Portions and RNAi
Agent Sense Strand Sequences

| Mouse | | |
|---|---|---|
| sequences | (Genbank accession number: NM_019388.1) | |
| CD86-194 | AGUAUUUUGGCAGGACCAG | (SEQ ID NO: 145) |
| CD86-488 | CAUAAAUUUGACCUGCACG | (SEQ ID NO: 146) |
| CD86-594 | CAAGAUAAUGUCACAGAAC | (SEQ ID NO: 147) |
| Human | | |
| sequences | (Genbank accession number: NM_006889.1) | |
| HCD86-291 | AGUAUUUUGGCAGGACCAG | (SEQ ID NO: 148) |
| HCD86-585 | CAUAAAUUUGACCUGCUCA | (SEQ ID NO: 149) |
| HCD86-697 | CAAGAUAAUGUCACAGAAC | (SEQ ID NO: 150) |

TABLE 11
RelA Target Portions and RNAi
Agent Sense Strand Sequences

| Mouse | | |
|---|---|---|
| sequences | (Genbank accession number: BC003818) | |
| RelA-255 | AGCACAGAUACCACCAAGA | (SEQ ID NO: 151) |
| RelA-282 | ACCAUCAAGAUCAAUGGCU | (SEQ ID NO: 152) |
| RelA-672 | AACACUGCCGAGCUCAAGA | (SEQ ID NO: 153) |
| RelA-682 | AGCUCAAGAUCUGCCGAGU | (SEQ ID NO: 154) |
| RelA-1707 | AUUGCGGACAUGGACUUCU | (SEQ ID NO: 155) |
| RelA-1735 | UGAGUCAGAUCAGCUCCUA | (SEQ ID NO: 156) |
| Human | | |
| sequences | (Genbank accession number: BC011603) | |
| HRelA-214 | AGCACAGAUACCACCAAGA | (SEQ ID NO: 157) |
| HRelA-241 | ACCAUCAAGAUCAAUGGCU | (SEQ ID NO: 158) |
| HRelA-631 | AACACUGCCGAGCUCAAGA | (SEQ ID NO: 159) |
| HRelA-641 | AGCUCAAGAUCUGCCGAGU | (SEQ ID NO: 160) |
| HRelA-1181 | AUUGCGGACAUGGACUUCU | (SEQ ID NO: 161) |
| HRelA-1209 | UGAGUCAGAUCAGCUCCUA | (SEQ ID NO: 162) |

TABLE 12
Re1B Target Portions and RNAi Agent Sense
Strand Sequences

| Mouse | | |
|---|---|---|
| sequences | (Genbank accession number: BC019765) | |
| RelB-794 | UUUAACAACCUGGGCAUCC | (SEQ ID NO: 163) |
| RelB-840 | CUGCCAUUGAGCGGAAGAU | (SEQ ID NO: 164) |
| RelB-1583 | CUCCUGGACGAUGGCUUUG | (SEQ ID NO: 165) |
| RelB-1788 | UUGUGGGCAGCAACAUGUU | (SEQ ID NO: 166) |
| Human | | |
| sequences | (Genbank accession number: BCO28013) | |
| HRelB-747 | UUUAACAACCUGGGCAUCC | (SEQ ID NO: 167) |
| HRelB-793 | CUGCCAUUGAGCGGAAGAU | (SEQ ID NO: 168) |
| HRelB-1533 | CUCCUGGACGAUGGCUUUG | (SEQ ID NO: 169) |
| HRelB-1738 | UUGUGGGCAGCAACAUGUU | (SEQ ID NO: 170) |

TABLE 13
4-1BBL Target Portions and RNAi Agent Sense
Strand Sequences

| Mouse | | |
|---|---|---|
| sequences | (Genbank accession number: L15435.1) | |
| 4-1BBL-309 | UAGUCGCUUUGGUUUUGCU | (SEQ ID NO: 171) |
| 4-1BBL-418 | AGAGAAUAAUGCAGACCAG | (SEQ ID NO: 172) |
| 4-1BBL-987 | UAUCCUUCUUGUGACUCCU | (SEQ ID NO: 173) |
| 4-1BBL-1016 | UCCUCAAGCUGCUAUGUUU | (SEQ ID NO: 174) |
| Human | | |
| sequences | (Genbank accession number: U03398.1) | |
| H4-1BBL-298 | AAUGUUCUGCUGAUCGAUG | (SEQ ID NO: 175) |
| H4-1BBL-374 | UGAGCUACAAAGAGGACAC | (SEQ ID NO: 176) |
| H4-1BBL-1019 | AGGAUCCUGAGUUUGUGAA | (SEQ ID NO: 177) |
| H4-1BBL-1207 | CUGUAAUGUGCCAGCAUUG | (SEQ ID NO: 178) |
| H4-1BBL-1240 | GGCUAUAGAAACAUCUAGA | (SEQ ID NO: 179) |
| H4-1BBL-1283 | UAUGGUAAUACGUGAGGAA | (SEQ ID NO: 180) |

TABLE 14
TLR1 Target Portions and RNAi Agent Sense
Strand Sequences

| Mouse | | |
|---|---|---|
| sequences | (Genbank accession number: AY009154.1) | |
| TLR1-468 | UUUGGAUUUGUCCCACAAU | (SEQ ID NO: 181) |
| TLR1-1698 | GGAUUUCUUCCAGAGCUGU | (SEQ ID NO: 182) |
| TLR1-2246 | GUUACAAGUCCAUCUUUGU | (SEQ ID NO: 183) |
| Human | (Genbank accession number: | |
| sequences | NM_003263.2) | |
| HTLR1-565 | CUUGGAUUUGUCCCACAAC | (SEQ ID NO: 184) |
| HTLR1-1795 | UGAUUUCUUCCAGAGCUGC | (SEQ ID NO: 185) |
| HTLR1-2343 | GUUACAAGUCCAUCUUUGU | (SEQ ID NO: 186) |

TABLE 15
TLR2 Target Portions and RNAi Agent Sense
Strand Sequences

| Mouse | | |
|---|---|---|
| sequences | (Genbank accession number: AF216289.1) | |
| TLR2-477 | GAAAAGCCUUGACCUGUCU | (SEQ ID NO: 187) |
| TLR2-2460 | CGAACUGGACUUCUCCCAC | (SEQ ID NO: 188) |
| Human | (Genbank accession number: | |
| sequences | NM_003264.2) | |
| HTLR2-312 | AAAAAGCCUUGACCUGUCC | (SEQ ID NO: 189) |
| HTLR2-2295 | UGAACUGGACUUCUCCCAU | (SEQ ID NO: 190) |

TABLE 16
TLR3 Target Portions and RNAi Agent Sense
Strand Sequences

| Mouse | (Genbank accession number: | |
|---|---|---|
| sequences | NM_126166.2) | |
| TLR3-1178 | GAAGUGGACAAAUCUCACC | (SEQ ID NO: 191) |
| TLR3-1711 | GCCAGGAAUGGAGAGGUCU | (SEQ ID NO: 192) |
| TLR3-1876 | CUCUUCGUAACUUGACCAU | (SEQ ID NO: 193) |
| TLR3-1904 | AAGCAACAACAACAUAGCC | (SEQ ID NO: 194) |
| TLR3-2046 | CUGUCUCACCUCCACAUCU | (SEQ ID NO: 195) |
| TLR3-2309 | CUGCACGUGUGAAAGUAUU | (SEQ ID NO: 196) |
| TLR3-2848 | GAAGAUUCAAGGUACAUCA | (SEQ ID NO: 197) |
| Human | (Genbank accession number: | |
| sequences | NM_003265.2) | |
| HTLR3-914 | AAAGUGGACAAAUCUCACU | (SEQ ID NO: 198) |
| HTLR3-1447 | GCCAGGAAUGGAGAGGUCU | (SEQ ID NO: 199) |
| HTLR3-1612 | CUCUUCGUAACUUGACCAU | (SEQ ID NO: 200) |
| HTLR3-1640 | AAGCAACAACAACAUAGCC | (SEQ ID NO: 201) |
| HTLR3-1782 | CUGUCUCACCUCCACAUCC | (SEQ ID NO: 202) |
| HTLR3-2045 | UUGCACGUGUGAAAGUAUU | (SEQ ID NO: 203) |
| HTLR3-2584 | AAAGAUUCAAGGUACAUCA | (SEQ ID NO: 204) |
| HTLR3-2682 | AACCAUGCACUCUGUUUGC | (SEQ ID NO: 205) |

TABLE 17
TLR4 Target Portions and RNAi Agent Sense
Strand Sequences

| Mouse | (Genbank accession number: | |
|---|---|---|
| sequences | NM_021297.1) | |
| TLR4-266 | UGGAUUUAUCCAGGUGUGA | (SEQ ID NO: 206) |
| TLR4-410 | UGGUGGCUGUGGAGACAAA | (SEQ ID NO: 207) |
| TLR4-2138 | ACUACAGAGACUUUAUUCC | (SEQ ID NO: 208) |
| TLR4-2169 | UUGCUGCCAACAUCAUCCA | (SEQ ID NO: 209) |
| Human | | |
| sequences | (Genbank accession number: U88880.1) | |
| HTLR4-412 | UGGAUUUAUCCAGGUGUGA | (SEQ ID NO: 210) |
| HTLR4-556 | UGGUGGCUGUGGAGACAAA | (SEQ ID NO: 211) |
| HTLR4-2287 | ACUACAGAGACUUUAUUCC | (SEQ ID NO: 212) |
| HTLR4-2317 | UUGCUGCCAACAUCAUCCA | (SEQ ID NO: 213) |

TABLE 18
TLR5 Target Portions and RNAi Agent Sense
Strand Sequences

| Mouse | (Genbank accession number: | |
|---|---|---|
| sequences | NM_016928.1) | |
| TLR5-1160 | CAGCUUCAACUAUAUCAGU | (SEQ ID NO: 214) |
| TLR5-3130 | CUUUGCUCAAACACCUGGA | (SEQ ID NO: 215) |
| Human | (Genbank accession number: | |
| sequences | NM_003268.3) | |
| HTLR5-800 | GAGCUUCAACUAUAUCAGG | (SEQ ID NO: 216) |
| HTLR5-2770 | CUUUGCUCAAACACCUGGA | (SEQ ID NO: 217) |

TABLE 19
TLR6 Target Portions and RNAi Agent Sense
Strand Sequences

| Mouse | (Genbank accession number: | |
|---|---|---|
| sequences | NM_011604.1) | |
| TLR6-550 | UUGCUCACUUGCAUCUAAG | (SEQ ID NO: 218) |
| TLR6-2005 | AUGAUUCUGCCUGGGUGAA | (SEQ ID NO: 219) |
| TLR6-2073 | CAUGAGAGGAACUUUGUCC | (SEQ ID NO: 220) |
| Human | (Genbank accession number: | |
| sequences | NM_006068.2) | |
| HTLR6-563 | UUGCUCACUUGCAUCUAAG | (SEQ ID NO: 221) |
| HTLR6-2018 | AUGAUUCUGCCUGGGUGAA | (SEQ ID NO: 222) |
| HTLR6-2086 | CAUGAGAGGAACUUUGUCC | (SEQ ID NO: 223) |

TABLE 20
TLR7 Target Portions and RNAi Agent Sense
Strand Sequence

| Mouse | (Genbank accession number: | |
|---|---|---|
| sequences | NM_133211.2) | |
| TLR7-272 | GAUGGUUUCCUAAAACUCU | (SEQ ID NO: 224) |
| TLR7-584 | UUUACCUGGAUGGAAACCA | (SEQ ID NO: 225) |
| TLR7-736 | UGUUAUUAUCGAAAUCCUU | (SEQ ID NO: 226) |
| TLR7-824 | AAGAUAACAAUGUCACAGC | (SEQ ID NO: 227) |
| TLR7-947 | UUCUUGACCUAAGUGGAAA | (SEQ ID NO: 228) |
| TLR7-1782 | CCAAACUCUUAAUGGCAGU | (SEQ ID NO: 229) |
| TLR7-2556 | CUGUGAUGCUGUGUGGUUU | (SEQ ID NO: 230) |
| TLR7-2706 | AAACCUGAUUCUGUUCUCA | (SEQ ID NO: 231) |
| Human | (Genbank accession number: | |
| sequences | NM_016562.2) | |
| HTLR7-218 | GAUGGUUUCCUAAAACUCU | (SEQ ID NO: 232) |
| HTLR7-530 | UUUACCUGGAUGGAAACCA | (SEQ ID NO: 233) |
| HTLR7-682 | UGUUAUUAUCGAAAUCCUU | (SEQ ID NO: 234) |
| HTLR7-770 | AAGAUAACAAUGUCACAGC | (SEQ ID NO: 235) |
| HTLR7-893 | UUCUUGACCUAAGUGGAAA | (SEQ ID NO: 236) |
| HTLR7-1728 | CCAAACUCUUAAUGGCAGU | (SEQ ID NO: 237) |
| HTLR7-2502 | CUGUGAUGCUGUGUGGUUU | (SEQ ID NO: 238) |
| HTLR7-2652 | AAACCUGAUUCUGUUCUCA | (SEQ ID NO: 239) |

TABLE 21
TLR8 Target Portions and RNAi Agent Sense
Strand Sequences

| Mouse | (Genbank accession number: | |
|---|---|---|
| sequences | NM_133212.1) | |
| TLR8-2589 | ACUGGGAUGUUUGGUUUAU | (SEQ ID NO: 240) |
| TLR8-2821 | UAACCUCAUGCAGAGCAUA | (SEQ ID NO: 241) |
| TLR8-2921 | UUGCAGAGGCUAAUGGAUG | (SEQ ID NO: 242) |
| TLR8-2939 | GAGAACAUGGAUGUGAUUA | (SEQ ID NO: 243) |
| Human | (Genbank accession number: | |
| sequences | NM_016610.2) | |
| HTLR8-2763 | ACUGGGAUGUUUGGUUUAU | (SEQ ID NO: 244) |
| HTLR8-2995 | CAACCUCAUGCAGAGCAUC | (SEQ ID NO: 245) |
| HTLR8-3095 | UUGCAGAGGCUAAUGGAUG | (SEQ ID NO: 246) |
| HTLR8-3113 | GAGAACAUGGAUGUGAUUA | (SEQ ID NO: 247) |

TABLE 22
TLR9 Target Portions and RNAi Agent Sense
Strand Sequences

| Mouse | | |
|---|---|---|
| sequences | (Genbank accession number: AF314224.1) | |
| TLR9-1103 | AACCUGUCCUUCAAUUACC | (SEQ ID NO: 248) |
| TLR9-1200 | ACGGCAUCUUCUUCCGCUC | (SEQ ID NO: 249) |
| TLR9-1283 | AUGAACUUCAUCAACCAGG | (SEQ ID NO: 250) |
| TLR9-2156 | AAGGCCCUGACCAAUGGCA | (SEQ ID NO: 251) |
| Human | (Genbank accession number: | |
| sequences | NM_017442.2) | |
| HTLR9-1652 | AACCUGUCCUUCAAUUACC | (SEQ ID NO: 252) |
| HTLR9-1749 | ACGGCAUCUUCUUCCGCUC | (SEQ ID NO: 253) |
| HTLR9-1832 | AUGAACUUCAUCAACCAGG | (SEQ ID NO: 254) |
| HTLR9-2702 | AAGGCCCUGACCAAUGGCA | (SEQ ID NO: 255) |

TABLE 23
FcεRα Target Portions and RNAi Agent Sense
Strand Sequences

| Mouse | (Genbank accession number: | |
|---|---|---|
| sequences | AJ245551.1) | |
| CD83-454 | GACACUCAUCAUUUUCACC | (SEQ ID NO: 256) |
| CD83-841 | GCUAUCUGGUCAACCUCGU | (SEQ ID NO: 257) |
| CD83-881 | AAGCUAUGGUGAGAUGCAG | (SEQ ID NO: 258) |
| CD83-955 | CUGAGGACAGCUGUCCUCU | (SEQ ID NO: 259) |
| CD83-1071 | CAGUGGGAAAUAUUUAGCA | (SEQ ID NO: 260) |
| CD83-1251 | CAUGUACUUGUCAAAGAAG | (SEQ ID NO: 261) |
| Human | (Genbank accession number: | |
| sequences | NM_004233.2) | |
| HCD83-779 | AACACUCAUCAUUUUCACU | (SEQ ID NO: 262) |
| HCD83-1176 | GCUAUCUGGUCAACCUCCU | (SEQ ID NO: 263) |
| HCD83-1217 | AAGCUAUGGUGAGAUGCAG | (SEQ ID NO: 264) |
| HCD83-1290 | CUGAGGACAGCUGUCCUCU | (SEQ ID NO: 265) |
| HCD83-1406 | CAGUGGGAAAUAUUUAGCA | (SEQ ID NO: 266) |
| HCD83-1584 | UAUGUACUUGUCAAAGAAG | (SEQ ID NO: 267) |

TABLE 24
SLAM Target Portions and RNAi Agent Sense
Strand Sequences

| Mouse | (Genbank accession number: | |
|---|---|---|
| sequences | NM_013730.1) | |
| SLAM-100 | UUUCUCUCCCUGGCUUUUG | (SEQ ID NO: 268) |
| SLAM-123 | GAGCUACGGAACAGGUGGA | (SEQ ID NO: 269) |
| Human | (Genbank accession number: | |
| sequences | AY040554.1) | |
| HSLAM-40 | UUUCUCUCCCUGGCUUUUG | (SEQ ID NO: 270) |
| HSLAM-63 | AAGCUACGGAACAGGUGGG | (SEQ ID NO: 271) |

TABLE 25
Common γ Chain Target Portions and RNAi Agent
Sense Strand Seqences

| Mouse | (Genbank accession number: | |
|---|---|---|
| sequences | NM-013563.1) | |
| IL-2Rγ-209 | GAGUACAUGAAUUGCACUU | (SEQ ID NO: 272) |
| IL-2Rγ-501 | UGAGUGAAUCCCAGCUAGA | (SEQ ID NO: 273) |
| IL-2Rγ-933 | CCUGGAGUGGUGUGUCUAA | (SEQ ID NO: 274) |
| IL-2Rγ-972 | AGCCAGACUACAGUGAACG | (SEQ ID NO: 275) |
| Human | (Genbank accession number: | |
| sequences | NM-000206.1) | |
| HIL-2Rγ-216 | GAGUACAUGAAUUGCACUU | (SEQ ID NO: 276) |
| HIL-2Rγ-508 | UGAGUGAAUCCCAGCUAGA | (SEQ ID NO: 277) |
| HIL-2Rγ-940 | CCUGGAGUGGUGUGUCUAA | (SEQ ID NO: 278) |
| HIL-2Rγ-979 | AGCCAGACUACAGUGAACG | (SEQ ID NO: 279) |

TABLE 26
FcεRα Target Portions and RNAi Agent Sense
Strand Sequences

| Mouse | | |
|---|---|---|
| sequences | (Genbank accession number: M94967.1) | |
| COX2-175 | CAGCAAAUCCUUGCUGUUC | (SEQ ID NO: 280) |
| COX2-232 | GAUUUGACCAGUAUAAGUG | (SEQ ID NO: 281) |
| COX2-337 | CAAACACAGUGCACUACAU | (SEQ ID NO: 282) |
| COX2-448 | AUUUGAUUGACAGUCCACC | (SEQ ID NO: 283) |
| COX2-489 | UACAAAAGCUGGGAAGCCU | (SEQ ID NO: 284) |
| COX2-681 | UUCUUUGCCCAGCACUUCA | (SEQ ID NO: 285) |
| COX2-714 | AAGACAGAUCAUAAGCGAG | (SEQ ID NO: 286) |
| COX2-809 | UAAACUGCGCCUUUUCAAG | (SEQ ID NO: 287) |
| COX2-818 | CCUUUUCAAGGAUGGAAAA | (SEQ ID NO: 288) |
| COX2-896 | AGAGAUGAUCUACCCUCCU | (SEQ ID NO: 289) |
| COX2-954 | GUCUUUGGUCUGGUGCCUG | (SEQ ID NO: 290) |
| COX2-973 | GUCUGAUGAUGUAUGCCAC | (SEQ ID NO: 291) |
| COX2-1433 | CUCCAUUGACCAGAGCAGA | (SEQ ID NO: 292) |
| COX2-1452 | GAGAUGAAAUACCAGUCUC | (SEQ ID NO: 293) |
| COX2-1473 | AAUGAGUACCGCAAACGCU | (SEQ ID NO: 294) |
| COX2-1516 | UUGAAGAACUUACAGGAGA | (SEQ ID NO: 295) |
| COX2-1657 | UUGGAGCACCAUUCUCCUU | (SEQ ID NO: 296) |
| COX2-1764 | ACUGCCUCAAUUCAGUCUC | (SEQ ID NO: 297) |
| Human | | |
| sequences | (Genbank accession number: M90100.1) | |
| HCOX2-147 | CAGCAAAUCCUUGCUGUUC | (SEQ ID NO: 298) |
| HCOX2-204 | GAUUUGACCAGUAUAAGUG | (SEQ ID NO: 299) |
| HCOX2-309 | CAAACACAGUGCACUACAU | (SEQ ID NO: 300) |
| HCOX2-420 | AUUUGAUUGACAGUCCACC | (SEQ ID NO: 301) |
| HCOX2-461 | UACAAAAGCUGGGAAGCCU | (SEQ ID NO: 302) |
| HCOX2-653 | UUCUUUGCCCAGCACUUCA | (SEQ ID NO: 303) |
| HCOX2-686 | AAGACAGAUCAUAAGCGAG | (SEQ ID NO: 304) |
| HCOX2-781 | UAAACUGCGCCUUUUCAAG | (SEQ ID NO: 305) |
| HCOX2-790 | CCUUUUCAAGGAUGGAAAA | (SEQ ID NO: 306) |
| HCOX2-868 | AGAGAUGAUCUACCCUCCU | (SEQ ID NO: 307) |
| HCOX2-926 | GUCUUUGGUCUGGUGCCUG | (SEQ ID NO: 308) |
| HCOX2-945 | GUCUGAUGAUGUAUGCCAC | (SEQ ID NO: 309) |
| HCOX2-1405 | UUCCAUUGACCAGAGCAGG | (SEQ ID NO: 310) |
| HCOX2-1424 | CAGAUGAAAUACCAGUCUU | (SEQ ID NO: 311) |
| HCOX2-1445 | AAUGAGUACCGCAAACGCU | (SEQ ID NO: 312) |
| HCOX2-1488 | UUGAAGAACUUACAGGAGA | (SEQ ID NO: 313) |
| HCOX2-1629 | UUGGAGCACCAUUCUCCUU | (SEQ ID NO: 314) |
| HCOX2-1736 | ACUGCCUCAAUUCAGUCUC | (SEQ ID NO: 315) |

### VII. Methods for Identification, Testing, and Selection of RNAi Agents that Reduce or Eliminate IgE-Mediated Hypersensitivity

The techniques and reagents described herein can readily be applied to design additional new RNAi agents, targeted to other genes or gene regions. These agents can be tested for their activity in inhibiting Ig-E mediated responses, diseases, and conditions as discussed herein. In various embodiments of the invention RNAi agents such as siRNAs or shRNAs are tested by first introducing candidate RNAi agents into cells (e.g., by exogenous administration or by introducing into the cell a vector or construct that directs endogenous synthesis of siRNA or shRNA). The ability of a candidate RNAi agent to reduce the level of the target transcript is then assessed by measuring the amount of the target transcript using, for example, Northern blots, nuclease protection assays, reverse transcription (RT)-PCR, real-time RT-PCR, microarray analysis, etc. The ability of a candidate RNAi agent to inhibit production of a polypeptide encoded by the target transcript (either at the transcriptional or post-transcriptional level) may be measured using a variety of antibody-based approaches including, but not limited to, Western blots, immunoassays, flow cytometry, protein microarrays, etc. In general, any method of measuring the amount of either the target transcript or a polypeptide encoded by the target transcript may be used. In general, certain preferred inhibitors reduce the target transcript level at least about 2 fold, preferably at least about 4 fold, more preferably at least about 8 fold, at least about 16 fold, at least about 64 fold or to an even greater degree relative to the level that would be present in the absence of the inhibitor (e.g., in a comparable control cell lacking the inhibitor).

The invention provides various additional methods of identifying RNAi agents such as siRNAs and shRNAs and testing their efficacy. For example, inventive RNAi agents may be tested to assess their effect in vitro on cellular responses such as mast cell degranulation in response to various stimuli such as IgE, Th2 cell response (e.g., proliferation, release of cytokines such as IL-3, IL-4, IL-5, 11-6, IL-10, and IL-13) in response to stimulation by DC, macrophages, B cells, or microglial cells, etc. Methods for performing such assays are well known in the art. In general, for any of the above tests, cells to which inventive RNAi agents have been delivered (test cells) may be compared with similar or comparable cells that have not received the inventive composition (control cells). Thus the invention provides a method of identifying an RNAi agent comprising a suitable sequence for treatment of a condition characterized by IgE-mediated hypersensitivity or excessive or inappropriate mast cell activity comprising: (i) delivering a candidate RNAi agent to mast cells either prior to, at the same time as, or after exposure to an appropriate stimulus; (ii) assessing the production or secretion of a mediator; (iii) comparing the amount of the mediator produced or secreted in the presence of the RNAi agent with the amount produced or secreted in the absence of the RNAi agent; and (iv) identifiying an RNAi agent as comprising a suitable sequence if the amount of the mediator produced or secreted in the presence of the RNAi agent is less than the amount of the mediator produced or secreted in the absense of the RNAi agent. The RNAi agent can be an siRNA, shRNA, or RNAi vector in various embodiments of the invention. See Example 2, which describes testing the ability of a candidate siRNA to inhibit mast cell degranulation.

Inventive RNAi agents can be administered to subjects, e.g., rodents, non-human primates, or humans, and cells such as mast cells, Th2 cells, DC, B cells, etc., can be harvested from the subject. The ability of inventive RNAi agents to inhibit expression of the target trancript and/or its encoded protein is measured as above. As mentioned above, certain RNAi agents are targeted to transcripts that encode proteins that contribute to and/or are necessary for mast cell survival and/or proliferation (e.g., c-Kit). The effect of the inventive RNAi agents on the number of mast cells can also be measured, where a decrease in the number of mast cells following administration of an inventive RNAi agent is an indication of its efficacy.

The invention further provides a method of identifying an RNAi agent comprising a sequence suitable for treatment of a condition characterized by IgE-mediated hypersensitivity or inappropriate or excessive Th2 helper cell activity comprising: (i) delivering the candidate RNAi agent to a culture comprising T cells and APCs; (ii) assessing T cell proliferation and/or assessing the production or secretion of a cytokine characteristic of Th2 cells; (iii) comparing the extent of T cell proliferation or the production or secretion of the cytokine in the presence of the RNAi agent with the extent of T cell proliferation or the production or secretion of the cytokine in the absence of the RNAi agent; and (iv) identifiying an RNAi agent as comprising a suitable sequence if the extent of T cell proliferation or the production or secretion of the cytokine in the presence of the RNAi agent is less than the extent of T cell proliferation or the production or secretion of the cytokine in the absence of the RNAi agent. (The tests may include a control in which the RNAi agent is absent but may also make use of previous information regarding the amount of mediator produced or secreted in the absence of inhibition or the amount of T cell proliferation or cytokine production or release in the absence of inhibition.) These assays may be used to test RNAi agents that target any transcript and is not limited to agents that target the transcripts described herein.

Certain of the inventive RNAi agents are targeted to transcripts that encode proteins that contribute to or are necessary for IgE production. The effect of the inventive RNAi agent on IgE production by B cells in cell culture or on IgE levels in a subject can be measured. A decrease in the level of IgE following administration of an inventive RNAi agent, or a reduced IgE response following administration of an antigen in the presence of the RNAi agent versus the expected IgE response in the absence of the RNAi agent is one indication of the efficacy of the RNAi agent. The invention provides a method of identifying an RNAi agent comprising a sequence suitable for treatment of a condition characterized by IgE-mediated hypersensitivity comprising: (i) delivering a candidate RNAi agent to a culture comprising B cells; (ii) assessing the production or secretion of IgE; (iii) comparing the amount of IgE produced or secreted in the presence of the RNAi agent with the amount produced or secreted in the absence of the RNAi agent; and (iv) identifiying an RNAi agent as comprising a suitable sequence if the amount of IgE produced or secreted in the presence of the RNAi agent is less than the amount of IgE produced or secreted in the absense of the RNAi agent. The invention further provides another method of identifying an RNAi agent comprising a sequence suitable for treatment of a condition characterized by IgE-mediated hypersensitivity comprising: (i) delivering a candidate RNAi agent to a subject; (ii) obtaining a value for an indicator of IgE-mediated hypersensitivity selected from the group consisting of: the level of serum IgE, proliferation of T cells, production of a cytokine characteristic of Th2 cells, airway inflammation, airway reactivity, airway wall remodeling, and pulmonary function; (iii) comparing the value obtained in the presence of the RNAi agent with the value obtained in the absence of the RNAi agent; and (iv) identifying an RNAi agent as comprising a suitable sequence if the value obtained in the presence of the RNAi agent is less than the value obtained in the absense of the RNAi agent.

It is noted that if the efficacy of an RNAi agent whose duplex portion comprises a particular sequence as a resulting in RNAi is established using one type of RNAi agent (e.g., an RNAi vector), the sequence will, in general, also be useful in the context of other types of RNAi agents, e.g., siRNAs or shRNAs. Thus, for example, if an RNAi vector such as a lentiviral vector reduces symptoms of asthma or allergic rhinitis in an animal model of such a condition, then an siRNA or shRNA having the same duplex portion as that for which the RNAi vector provides a template will, in general, also be useful for reducing symptoms of asthma or allergic rhinitis. Therefore, the methods above are described in terms of identifying an RNAi agent comprising a suitable sequence (i.e., duplex portion sequence) rather than in terms of identifying the effective RNAi agent itself. However, it is to be understood that identification of an RNAi agent comprising a suitable sequence essentially results in identification of the RNAi agent itself and also of RNAi agents having a duplex portion with the same sequence.

Potential inhibitory RNAi agents can be tested using any of variety of animal models for allergy and/or asthma that have been developed, e.g., rodent, sheep, or non-human primate models. See Isenberg-Feig, H., et al., “Animal models of allergic asthma”, Curr Allergy Asthma Rep., 3(1):70-8, 2003, and references therein, for examples of suitable animal models that are useful for testing the therapeutics of the present invention. See also Wegner C D, Gundel R G, Abraham W M, et al., J Allergy Clin Immunol, 91:917-29, 1993; Temelkovski, J., et al., Thorax, Volume 53(10): 849-856, 1998. Many such models are based upon sensitization by systemic administration of protein antigens such as ovalbumin and subsequent inhalational challenge followed by evaluation of various responses. These include indicators such as the level of serum IgE specific for the antigen, proliferation of antigen-specific T cells, airway inflammation (e.g, accumulation of inflammatory cells such as lymphocytes, neutrophils, and eosophinils in the airways), airway reactivity (e.g., in response to methacholine challenge), airway wall remodeling (e.g., airway thickening), and pulmonary function. RNAi agents targeted to TLR, e.g., TLR4, can be tested in any of a variety of animal models for sepsis, shock, or burn-related injury.

Compositions comprising candidate siRNA(s), shRNA(s) constructs or vectors capable of directing synthesis of such siRNAs or shRNAs within a host cell (i.e., comprising templates for transcription of the siRNA or shRNA, operably linked to appropriate expression signals), or cells engineered or manipulated to contain candidate RNAi agents may be administered to a human or animal subject prior to, simultaneously with, or following exposure to an antigen or in the absence of known exposure. The ability of the composition to prevent or reduce IgE-mediated hypersensitivity and/or to delay or prevent appearance of symptoms related to conditions and diseases associated with such hypersensitivity (e.g., allergic rhinitis and asthma) and/or lessen their severity relative to comparably exposed subjects that have not received the potential inhibitor is assessed.

As described above, a number of RNAi agents targeted to a variety of proteins important in IgE-mediated responses have been designed. The availability of a few potent inhibitory agents will facilitate their optimal use in combinations. For example, RNAi agents targeted to different transcripts may have a synergistic effect, i.e., an effect greater than the sum of the individual effects, e.g., by inhibiting multiple pathways leading to IgE production and/or response to IgE or by inhibiting pathways in multiple cell types involved in IgE-mediated responses. Thus, RNAi agents can be tested in combinations of two or more so as to find the most effective combinations.

On the other hand, in order to avoid unwanted side effects, it may be desirable to utilize RNAi agents that produce less than maximum inhibition of expression of their target transcript. Therefore, the invention encompasses the systematic testing of RNAi agents targeted to the transcripts described above, alone or in combination. According to one approach, nonoverlapping siRNAs or shRNAs whose sequences span the entire transcript are synthesized and tested in vitro in cells or cell lines as described above and/or in vivo in animal models such as the allergic mouse. In addition, the potencies of siRNAs or shRNAs can be compared by titrating the amount of RNA used for transfection. For example, different amounts of inventive RNAs (such as 0.025, 0.05, 0.1, and 0.25 nmol), either individually or in combinatins, can be transfected or electroporated into mast cells, and degranulation (release of mediators such as histamine, prostaglandins, arachidonic acid, etc.) in response to stimuli can be measured (see Example 2 for details). The ability of inventive RNAi agents to reduce or eliminate Th2 response may also be measured either in vitro, e.g., in a mixed culture of T cells and DCs, or in vivo. The efficacy of the RNAi agents may also be assessed using a murine model of allergic airway inflammation. Varying amounts of RNAi agents may also be administered to sensitized mice. The response to antigen challenge in these mice may be assessed in a variety of ways, including by measuring expression of inflammatory cytokines (e.g., MIP-1α, MIP-1β, IL-4, IL-5, IL-13, etc.), by measuring the numbers of eosinophils and neutrophils in the lungs, and by performing pulmonary function tests (see Example 3 for details). Results from such experiments will help to determine not only the relative potencies of each RNAi agent but also the minimal amount necessary for maximal inhibition. The latter is useful for determining how much of each should be used in combinations.

### VIII. RNAi Compositions Comprising Delivery Agents

The inventors have recognized that effective RNAi therapy in general, including prevention and therapy of conditions and diseases associated with IgE-mediated reactions will be enhanced by efficient introduction of RNAi agents such as siRNAs, shRNAs, and RNAi vectors into cells. According to certain embodiments of the invention, RNAi agents are administered to cells in the respiratory tract or to cells lining other mucosal surfaces. In general, RNAi agents may be delivered to any site in or on the body, including but not limited to, locations where mast cell degranulation or cellular interaction between APCs and IgE-producing B cells may occur, or in any location in which mast cells, basophils, APCs, IgE-producing B cells, and/or Th2 cells may occur. The invention therefore provides compositions comprising any of a variety of non-viral delivery agents for enhanced delivery of siRNA, shRNA, and/or RNAi vectors to cells in intact organisms, e.g., mammals. As used herein, the concept of “delivery” includes transport of an siRNA, shRNA, or RNAi vector from its site of entry into the body to the location of the cells in which it is to function, in addition to cellular uptake of the siRNA, shRNA, or vector and any subsequent steps involved in making siRNA or shRNA available to the intracellular RNAi machinery (e.g., release or siRNA or shRNA from endosomes).

The invention therefore encompasses compositions comprising (i) an RNAi agent such as an siRNA or shRNA targeted to any of the transcripts discussed above, and/or an RNAi vector whose presence within a cell results in production of an RNAi agent such as an siRNA or shRNA that is targeted to any of the transcripts discussed above; and (ii) any of a variety of delivery agents including, but not limited to, cationic polymers, modified cationic polymers, peptide molecular transporters (including arginine or histidine-rich peptides), carbohydrates, lipids (including cationic lipids, neutral lipids, and combinations thereof), liposomes, lipopolyplexes, non-cationic polymers, surfactants suitable for introduction into the lung, or mixtures of any of the foregoing, etc. Certain of the delivery agents incorporate a moiety that increases delivery or increases the selective delivery of the RNAi agent or vector to cells in which it is desired to inhibit the transcript. In certain embodiments of the invention the delivery agent is biodegradable. Certain of the delivery agents suitable for use in the present invention are described below and in co-pending U.S. patent application Ser. No. 10/674,087, entitled “Compositions and Methods for Delivery of Short Interfering RNA and Short Hairpin RNA to Mammals”. The delivery agents may be used in combination.

Cationic polymer-based systems have been investigated as carriers for DNA transfection (Han, S.-0., et al., Mol. Therapy. 2:302-317, 2000). The ability of cationic polymers to promote intracellular uptake of DNA is thought to arise partly from their ability to bind to DNA and condense large plasmid DNA molecules into smaller DNA/polymer complexes for more efficient endocytosis. The DNA/cationic polymer complexes also act as bioadhesives because of their electrostatic interaction with negatively charged sialic acid residues of cell surface glycoproteins (Soane, R. J., et al., Int. J. Pharm. 178:55-65, 1999).

In addition, some polymers, such as imidazole group-modified polylysine (PLL), apparently promote disruption of the endosomal membrane and therefore release of DNA into the cytosol (Putnam, D., et al., Proc. Natl. Acad. Sci. USA 98:1200-1205, 2000). The invention therefore provides compositions comprising at least one RNAi agent and a cationic polymer and methods of inhibiting target gene expression by administering such compositions. The RNAi agent is targeted to a transcript that encodes a protein or peptide whose inhibition results in a decrease in IgE-mediated hypersensitivity, e.g., a transcript that encodes a protein or peptide whose inhibition results in any of the following: (1) a decrease in IgE production by B cells; (2) a decrease in mast cell number; (3) a decrease in mast cell activation; (4) a decrease in Th2 cell number, e.g., a decrease in allergen-specific Th2 cell number where the allergen is one that triggers hypersensitivity in a subject; (5) a decrease in Th2 cell activation, e.g., a decrease in activation of allergen-specific Th2 cells where the allergen is one that triggers hypersensitivity in a subject. According to certain embodiments of the invention administration of the RNAi agent results in decreased expression of a protein selected from the group consisting of: the FCεRIα chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2. According to certain embodiments of the invention expression is inhibited in DC and/or macrophages. According to certain embodiments of the invention the RNAi agent is targeted to a transcript encoding one of the foregoing proteins, resulting in a decrease in expression of the protein. However, according to other embodiments of the invention the RNAi agent is targeted to some other transcript whose encoded product is needed for or contributes to expression or activity of any of the proteins mentioned above. Such products include, e.g., transcription factors or RNA processing factors involved in transcription or processing of a transcript that encodes one of the foregoing proteins. Inventive RNAi agents may be administered individually or in combination with one another and/or in combination with other therapies for the treatment of diseases or conditions associated with IgE-mediated hypersensitivity.

The invention provides a variety of RNAi agents as described above and compositions comprising them. In particular, the invention provides methods of treating and/or preventing conditions and diseases associated with IgE-mediated hypersensitivity comprising administering a composition comprising (i) an RNAi agents that targets a transcript that encodes a protein selected from the group consisting of: the FCεRIα chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2 and (ii) a cationic polymer. The invention provides a variety of such RNAi agents and compositions comprising them.

In general, a cationic polymer is a polymer that is positively charged at approximately physiological pH, e.g., a pH ranging from approximately 7.0 to 7.6, preferably approximately 7.2 to 7.6, more preferably approximately 7.4. Such cationic polymers include, but are not limited to, imidazole group-modified PLL (Putnam, et al.), polyethyleneimine (PEI) (Boussif, O., et al., Proc. Natl. Acad. Sci. USA 92:7297-7301, 1995), polyvinylpyrrolidone (PVP) (Astafieva, I., et al., FEBS Lett. 389:278-280, 1996), and chitosan (Davis, S. S., Pharm. Sci. Technol. Today 2:450-457, 1999; Roy, K., et al., Nat. Med. 5:387-391, 1999).

It will be appreciated that certain of these polymers comprise primary amine groups, imine groups, guanidine groups, and/or imidazole groups. Preferred cationic polymers have relatively low toxicity and high DNA transfection efficiency. Preferred cationic polymers have relatively low toxicity and high DNA transfection efficiency.

Suitable cationic polymers also include copolymers comprising subunits of any of the foregoing polymers, e.g., lysine-histidine copolymers, etc. The percentage of the various subunits need not be equal in the copolymers but may be selected, e.g., to optimize such properties as ability to form complexes with nucleic acids while minimizing cytotoxicity. Furthermore, the subunits need not alternate in a regular fashion. Appropriate assays to evaluate various polymers with respect to desirable properties are described in the Examples. Preferred cationic polymers also include polymers such as the foregoing, further incorporating any of various modifications. Appropriate modifications include, but are not limited to, modification with acetyl, succinyl, acyl, or imidazole groups. In general, certain preferred modifications result in a reduction in the positive charge of the cationic polymer. Certain preferred modifications convert a primary amine into a secondary amine. Methods for modifying cationic polymers to incorporate such additional groups are well known in the art. (See, e.g., reference 32). For example, the c-amino group of various residues may be substituted, e.g., by conjugation with a desired modifying group after synthesis of the polymer. In general, it is desirable to select a % substitution sufficient to achieve an appropriate reduction in cytotoxicity relative to the unsubstituted polymer while not causing too great a reduction in the ability of the polymer to enhance delivery of the RNAi agent. Accordingly, in certain embodiments of the invention between 25% and 75% of the residues in the polymer are substituted. In certain embodiments of the invention approximately 50% of the residues in the polymer are substituted. It is noted that similar effects may be achieved by initially forming copolymers of appropriately selected monomeric subunits, i.e., subunits some of which already incorporate the desired modification.

While not wishing to be bound by any theory, it is believed that cationic polymers such as PEI compact or condense DNA into positively charged particles capable of interacting with anionic proteoglycans at the cell surface and entering cells by endocytosis. Such polymers may possess the property of acting as a “proton sponge” that buffers the endosomal pH and protects DNA from degradation. Continuous proton influx also induces endosome osmotic swelling and rupture, which provides an escape mechanism for DNA particles to the cytoplasm. (See, e.g., references 85-87; U.S. Pat. No. 6,013,240; WO9602655 for further information on PEI and other cationic polymers useful in the practice of the invention) According to certain embodiments of the invention the commercially available PEI reagent known as jetPEI™ (Qbiogene, Carlsbad, Calif.), a linear form of PEI (U.S. Pat. No. 6,013,240) is used.

The inventors have shown that compositions comprising PEI, PLL, or PLA and an siRNA that targets an influenza virus RNA significantly inhibit production of influenza virus in mice when administered intravenously either before or after influenza virus infection. The inhibition is dose-dependent and exhibits additive effects when two siRNAs targeted to different influenza virus RNAs were used. Thus siRNA, when combined with a cationic polymer such as PEI, PLL, or PLA, is able to reach the lung, to enter cells, and to effectively inhibit the viral replication cycle. These findings suggest that similar compositions containing siRNAs targeted against other transcripts expressed in the lung will be efficiently delivered to cells in the lung and inhibit expression of their target genes.

A variety of additional cationic polymers may also be used. Large libraries of novel cationic polymers and oligomers from diacrylate and amine monomers have been developed and tested in DNA transfection. These polymers are referred to herein as poly(β-amino ester) (PAE) polymers. For example, a library of 140 polymers from 7 diacrylate monomers and 20 amine monomers has been described (Lynn, D. M., et al., J. Am. Chem. Soc. 123:8155-8156, 2001) and larger libraries can be produced using similar or identical methodology. Of the 140 members of this library, 70 were found sufficiently water-soluble (2 mg/ml, 25 mM acetate buffer, pH=5.0). Fifty-six of the 70 water-soluble polymers interacted with DNA as shown by electrophoretic mobility shift. Most importantly, two of the 56 polymers mediated DNA transfection into COS-7 cells. Transfection efficiencies of the novel polymers were 4-8 times higher than PEI and equal or better than Lipofectamine 2000. The invention therefore provides compositions comprising at least one siRNA molecule and a cationic polymer, wherein the cationic polymer is a poly(β-amino ester), and methods of inhibiting target gene expression by administering such compositions.

Studies have shown that transcription factors, including HIV Tat protein (27, 28), VP22 protein of herpes simplex virus (29), and antennapedia protein of Drosophila (30), can penetrate the plasma membrane from the cell surface. The peptide segments responsible for membrane penetration consist of 11-34 amino acid residues and are highly enriched for arginine, referred to as arginine rich peptides (ARPs). When covalently linked with much larger polypeptides, the ARPs are capable of transporting the fused polypeptide across the plasma membrane (31-33). Similarly, when oligonucleotides were covalently linked to ARPs, they were much more rapidly taken up by cells (34, 35). Recent studies have shown that a polymer of eight arginines is sufficient for this transmembrane transport (36). Like cationic polymers, ARPs and polyarginine (PLA) are also positively charged and likely capable of binding siRNA, suggesting that it is probably not necessary to covalently link siRNA to ARPs or PLAs.

The invention therefore provides compositions comprising at least one RNAi agent and an arginine-rich peptide and methods of inhibiting target gene expression by administering such compositions. In particular, the invention provides methods of treating and/or preventing conditions or disorders associated with IgE-mediated hypersensitivity comprising administering a composition comprising (i) an RNAi agent that targets a transcript that encodes any encodes a protein or peptide whose inhibition results in a decrease in IgE-mediated hypersensitivity; and (ii) an arginine-rich peptide. According to certain embodiments of the invention administration of the RNAi agent is targeted to a transcript that encodes a protein selected from the group consisting of: the FCεR1α chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2. According to certain embodiments of the invention expression is inhibited in DC and/or macrophages. Arginine-rich peptides include, but are not limited to, those described in references 46-51 and variations thereof evident to one of ordinary skill in the art. Arginine-rich peptides include polyarginine (i.e., a peptide consisting of arginine residues only).

Generally, preferred arginine-rich peptides are less than approximately 50 amino acids in length. According to certain embodiments of the invention the arginine-rich peptide is a peptide having length between approximately 7 and 34 amino acids. According to certain embodiments of the invention a peptide is arginine-rich if it includes at least 20%, or at least 30%, or at least 40%, or at least 50%, or at least 60% or at least 70%, or at least 80%, or at least 90% arginine. According to certain embodiments of the invention the arginine-rich peptide includes between 6 and 20 arginine residues (i.e., the arginine-rich peptide includes 6 arginines or includes 7 arginines, or includes 8 arginines, etc.). According to certain embodiments of the invention the arginine-rich peptide (polyarginine) consists of between 6 and 20 arginine residues (i.e., the arginine-rich peptide includes 6 arginines or includes 7 arginines, or includes 8 arginines, etc.). According to certain embodiments of the invention the siRNA and the arginine-rich peptide are covalently bound, whereas in other embodiments of the invention the RNAi agent and the arginine-rich peptide are mixed together but are not covalently bound to one another.

A variety of other delivery agents may be used in various embodiments of the invention. For example, as described in more detail in U.S. patent application Ser. No. 10/674,087, surfactants suitable for introduction into the lung, delivery agents incorporating delivery-enhancing moieties such as antibodies or ligands that bind to molecules present on the surface of target cells, or any of a variety of polymers and polymer matrices distinct from the cationic polymers described above may also be used. Such polymers include a number of non-cationic polymers, i.e., polymers not having positive charge at physiological pH. Such polymers may have certain advantages, e.g., reduced cytotoxicity and, in some cases, FDA approval. A number of suitable polymers have been shown to enhance drug and gene delivery in other contexts. Such polymers include, for example, poly(lactide) (PLA), poly(glycolide) (PLG), and poly(DL-lactide-co-glycolide) (PLGA), which can be formulated into nanoparticles for delivery of inventive RNAi agents. Copolymers and combinations of the foregoing may also be used. In certain embodiments of the invention a cationic polymer is used to condense the siRNA, shRNA, or vector, and the condensed complex is protected by PLGA or another non-cationic polymer. Other polymers that may be used include noncondensing polymers such as polyvinyl alcohol, or poly(N-ethyl-4-vinylpyridium bromide, which may be complexed with Pluronic 85. Other polymers of use in the invention include combinations between cationic and non-cationic polymers. For example, poly(lactic-co-glycolic acid) (PLGA)-grafted poly(L-lysine) and other combinations including PLA, PLG, or PLGA and any of the cationic polymers or modified cationic polymers such as those discussed above, may be used.

### IX. Therapeutic Applications

Compositions containing inventive RNAi agents of the present invention may be used to prevent or treat any disease or condition mediated by IgE, e.g., any disease or condition associated with IgE-mediated hypersensitivity including, but not limited to, allergic rhinitis and asthma. Preferably, the amount of RNAi agent is sufficient to reduce or prevent one or more symptoms of IgE-mediated hypersensitivity. The invention therefore provides a method of treating or preventing a disease or condition characterized by IgE-mediated hypersensitivity, the method comprising steps of: (i) providing a subject at risk of or suffering from a disease or condition characterized by IgE-mediated hypersensitivity; and (ii) administering to the subject a composition comprising an RNAi agent targeted to a transcript encoding a protein selected from the group consisting of the FCεR1a chain, the FCεRIβ chain, c-Kit, Lyn, Syk, ICOS, OX40L, CD40, CD80, CD86, RelA, Re1B, 4-1BB ligand, TLR1, TLR2, TLR3, TLR4, TLR5, TLR6, TLR7, TLR8, TLR9, CD83, SLAM, common γ chain, and COX-2. The invention further provides a method of treating or preventing a disease or condition characterized by inappropriate or excessive mast cell activity or IgE-mediated hypersensitivity comprising steps of: providing a subject at risk of or suffering from a disease or condition characterized by inappropriate or excessive mast cell activity; and administering to the subject an RNAi agent composition that reduces mast cell activity or mast cell survival. In addition, the invention provides a method of treating or preventing a disease or condition characterized by inappropriate or excessive Th2 helper cell responses or IgE-mediated hypersensitivity comprising steps of: providing a subject at risk of or suffering from a disease or condition characterized by inappropriate or excessive Th2 helper cell responses; and administering to the subject an composition comprising an RNAi agent that reduces or eliminates Th2 cell response.

Inventive compositions containing an RNAi agent may contain a single species, targeted to a single site in a single target transcript, or alternatively may contain a plurality of different species, targeted to one or more sites in one or more target transcripts.

In some embodiments of the invention, it will be desirable to utilize compositions containing collections of different RNAi agents targeted to different transcripts. For example, it may be desirable to use a variety of RNAi agents targeted to transcripts expressed in different cell types, e.g., DC, macrophages, B cells, Th2 cells. Alternately, it may be desirable to inhibit a number of different transcripts in a single cell type. Either of these strategies may provide a therapeutic benefit while allowing a reduced level of inhibition of any single transcript relative to the degree of inhibition that would be needed to achieve an equivalent therapeutic effect if only a single transcript were inhibited.

According to certain embodiments of the invention, inventive compositions may contain more than one RNAi agent targeted to a single transcript. To give but one example, it may be desirable to include at least one siRNA or shRNA targeted to coding regions of a target transcript and at least one siRNA or shRNA targeted to the 3′ UTR. This strategy may provide extra assurance that products encoded by the relevant transcript will not be generated because at least one siRNA or shRNA in the composition may target the transcript for degradation while at least one other inhibits the translation of any transcripts that avoid degradation.

The invention encompasses “therapeutic cocktails”, including, but not limited to, approaches in which multiple siRNA or shRNAs are administered and approaches in which a single vector directs synthesis of siRNAs or shRNAs that inhibit multiple targets or of RNAs that may be processed to yield a plurality of siRNAs or shRNAs.

It will often be desirable to combine the administration of inventive RNAi agents with one or more other therapeutic agents in order to inhibit, reduce, or prevent one or more symptoms or characteristics of IgE-mediated hypersensitivity. In certain preferred embodiments of the invention, the inventive RNAi agents are combined with one or more other agents including, for example, antihistamines, including H1 receptor antagonists such as fexofenadine, loratadine, cetirizine, etc.; corticosteroids such as prednisone, beclamethasone, triamcinolone, fluticasone, etc.; bronchodilators including β-adrenergic agonists such as epinephrine, epinephrine analogs, and isoproterenol and 132-selective adrenergic agonists such as albuterol, metaproteronol, salmeterol, etc.; cromolyn sodium, nedocromil, or related compounds; methylxanthines such as theophylline or related compounds, etc. It is noted that the foregoing list is intended to be representative only rather than inclusive. See, e.g., Goodman and Gilman's Pharmacological Basis of Therapeutics, referenced above, for additional information and other suitable agents. In different embodiments of the invention the terms “combined with” or “in combination with” may mean either that the RNAi agent is present in the same mixture as the other agent(s) or that the treatment regimen for an individual includes both one or more RNAi agents and the other agent(s), not necessarily delivered in the same mixture or at the same time. According to certain embodiments of the invention the agent is approved by the U.S. Food and Drug Administration for the treatment of a condition associated with IgE-mediated hypersensitivity such as asthma or allergic rhinitis.

In some embodiments of the invention it may be desirable to target administration of inventive compositions to particular cells and/or cell types, e.g., mast cells, DC, macrophages, Th2 cells. In some embodiments of the invention it may be desirable to target administration of inventive compositions to particular regions of the body, e.g., the upper and/or lower airways, etc. In other embodiments of the invention it will be desirable to have available the greatest breadth of delivery options.

As noted above, inventive therapeutic protocols may involve administering an effective amount of an RNAi agent prior to, simultaneously with, or after exposure to an allergen to which the subject is hypersensitive. For example, individuals may receive an siRNA prior to an anticipated exposure or can be treated substantially contemporaneously with a suspected or known exposure (e.g., within seconds, minutes, or hours). Of course individuals may receive inventive treatment at any time including on an ongoing or routine basis.

Gene therapy protocols may involve administering an effective amount of a gene therapy vector comprising a template for transcription of an inhibitory siRNA or shRNA, operably linked to appropriate expression signals, to a subject. Another approach that may be used alternatively or in combination with the foregoing is to isolate a population of cells, e.g., stem cells or immune system cells from a subject, optionally expand the cells in tissue culture, and administer such a gene therapy vector to the cells in vitro. The cells may then be returned to the subject. Optionally, cells expressing the siRNA or shRNA can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. they can cross the epithelial barrier and enter into general circulation, thereby transfecting cells at other locations.

The present invention includes the use of inventive compositions for the treatment of nonhuman species including, but not limited to, dogs, cats, bovines, ovines, swine, and horses.

In preferred embodiments he gene therapy compositions and methods of the invention do not encompass claims to human beings or cells that form part of a human being.

### X. Pharmaceutical Formulations

Inventive compositions may be formulated for delivery by any available route including, but not limited to parenteral (e.g., intravenous), intramuscular, intradermal, subcutaneous, oral, nasal, bronchial, opthalmic, transdermal (topical), transmucosal, rectal, and vaginal routes. Preferred routes of delivery include parenteral, transmucosal, nasal, bronchial, and oral. Inventive pharmaceutical compositions typically include an siRNA or shRNA or vector that will result in production of an siRNA or shRNA after delivery, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to adjust isotonicity, e.g., by including agents such as, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive RNAi agents are preferably delivered in the form of an aerosol spray from a pressured container or dispenser which contains a suitable propellant, e.g., a gas such as carbon dioxide, or a nebulizer. The present invention particularly contemplates delivery of inventive compositions using a nasal spray, inhaler, or other direct delivery to the upper and/or lower airway. In addition, according to certain embodiments of the invention carriers to facilitate nucleic acid uptake by cells in the airway are included in the pharmaceutical composition. (See, e.g., S.-O. Han, R. I. Mahato, Y. K. Sung, S. W. Kim, “Development of biomaterials for gene therapy”, Molecular Therapy 2:302317, 2000.) According to certain embodiments of the invention the siRNAs or siRNA/carrier compositions are formulated as large porous particles for aerosol administration as described in more detail in Example 3.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, with the aid of high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an RNAi agent as described herein, can include a single treatment or, in many cases, can include a series of treatments.

Exemplary doses include milligram or microgram amounts of the inventive siRNA per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses of an RNAi agent depend upon its potency and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

As mentioned above, the present invention includes the use of inventive compositions for treatment of nonhuman animals. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

Plasmids or gene therapy vectors can be delivered to a subject by, for example, intravenous injection, local administration, or by stereotactic injection (see e.g., Chen et al. (1994) Proc. Natl. Acad. Sci. USA 91:3054-3057). In certain embodiments of the invention plasmids or gene therapy vectors may be delivered orally or inhalationally and may be encapsulated or otherwise manipulated to protect them from degradation, enhance uptake into tissues or cells, etc. Note that plasmids can be used as gene therapy vectors, and the term “gene therapy vector” can therefore encompass plasmids. However, in general, the term “gene therapy vector” is often used to refer to vectors that are able to provide more sustained expression of a therapeutic agent than typically provided when a naked DNA vector is introduced into mammalian cells, e.g., by replicating within cells and/or by causing integration of a nucleic acid sequence into the cellular genome. The pharmaceutical preparation of the plasmid or gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral or lentiviral vectors, the pharmaceutical preparation can include one or more cells which produce the gene delivery system.

Inventive pharmaceutical compositions can be included in a container, pack, or dispenser together with instructions for administration.

### EXAMPLES

### Example 1

### Design of siRNAs

The sequences listed in Tables 1-26 were selected as targets and as sense strands. For each sequence, a sequence perfectly complementary to the listed sequence was chosen as the corresponding antisense strand. A two nt 3′ overhang consisting of dTdT was added to each strand. For example, to design an siRNA based on the cDNA sequence FCεRα-268 (TTGGTCATTGTGAGTGCCA=SEQ ID NO: 316), the sequence 5′-UUGGUCAUUGUGAGUGCCA-3′ (SEQ ID NO: 1) is selected as the core region of the sense strand, and a complementary sequence, 5′-UGGCACUCACAAUGACCAA-3′ (SEQ ID NO: 317), is selected as the core region of the antisense strand. A two nt 3′ overhang consisting of dTdT is added to each strand, resulting in the sequences 5′-UUGGUCAUUGUGAGUGCCAdTdT-3′ (SEQ ID NO: 318) (sense strand) and 5′-UGGCACUCACAAUGACCAAdTdT-3′ (SEQ ID NO: 319) (antisense strand).

Hybridization of the sense and antisense strands results in an siRNA having a 19 base pair core duplex region, with each strand having a 2 nucleotide 3′ OH overhang.

### Example 2

### Effect of Inventive siRNAs on Antigen-Induced Mast Cell Responses

This example describes an experiment to determine the effect of administration of inventive siRNA compositions on release of various mediators of inflammation by basophils and mast cells in response to antigen.

Reagents. Unless otherwise stated, reagents are obtained from the sources described in Moriya, K., et al., Proc. Natl. Acad. Sci. USA, 94: 12539-12544, 1997.

Cells, cell culture, and cell preparation. RBL-2H3 is a basophilic leukemia cell line possessing high affinity IgE receptors. These cells can be activated to secrete histamine and other mediators by aggregation of these receptors or with calcium ionophores Barsumian E L, et al., Eur. J. Immunol. 11: 317-323, 1981. They have been used extensively to study FcERI and the biochemical pathways for secretion in mast cells. RBL-2H3 cells (line CRL-2256) are obtained from the American Type Culture Collection (Manassas, Va., http://www.atcc.org) and maintained in culture as described in Moriya, K., et al. RBL-2H3 cells are incubated overnight with DNP-specific IgE and radiolabelled myo-inositol, arachidonic acid, and 5-hydroxytryptamine as described in Moriya, et al. Cells are stimulated with antigen (DNP-BSA, 10 ng/ml) or the secretion-stimulating agents A23187 and phorbol 12-myristate β-acetate (100 nm and 20 M, respectively) for 15 min. at 37 degrees C.

Rat peritoneal mast cells are obtained as described in Holgate, S. T., et al., J. Immunol., 124: 2093-2099, 1980. Cells are stimulated by incubation with DNP-BSA (0.3 μg/ml) for 15 min. at 37 degrees C. Cultured human mast cells are obtained by the method described in Saito, et al., Int. Arch. Allergy Immunol., 107: 63-65, 1995. Cells are maintained in culture as described in Moriya, et al. For sensitization, human mast cells are incubated overnight with human IgE and radiolabelled arachidonic acid and are then stimulated with anti-human IgE as described (Moriya, et al.).

siRNAs. siRNAs are designed as described above. In addition to conforming to the selection criteria described in the Detailed Description with respect to GC content and the exclusion of strings of consecutive identical nucleotides, the siRNAs were generally designed in accordance with principles described in Technical Bulletin # 003-Revision B, “siRNA Oligonucleotides for RNAi Applications”, Dharmacon Research, Inc., Lafayette, Colo. 80026, a commercial supplier of RNA reagents. Technical Bulletins #003. Selected siRNAs correspond to portions of sequence that are identical in multiple species, e.g., humans and one or more rodents (e.g., mouse, rat) in order to facilitate testing efficacy in rodent cell lines and animal models.

All siRNAs are synthesized by Dharmacon Research (Lafayette, Colo.) using 2′ACE protection chemistry. The siRNA strands are deprotected according to the manufacturer's instructions, mixed in equimolar ratios and annealed by heating to 95° C. and slowly reducing the temperature by 1° C. every 30 s until 35° C. and 1° C. every min until 5° C.

siRNA administration. siRNA compositions comprising siRNAs targeted to the FCεRIα chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2, either alone or in combination are introduced into RBL-2H3 cells, rat peritoneal mast cells, and human mast cells using a liposome transfection approach as described in Li, L., et al., J. Biol. Chem., 278(7): 4725-4729, 2003. Alternately, cells are electroporated with siRNAs as described in U.S. Ser. No. 60/446,377.

Measurement of mediator release. The accumulation of total labeled inositol phosphates and arachidonic acid and 5-HT is determined as described in Cunha-Melho, J. R., et al., J. Immunol., 143:2617-2625, 1989, and Collado-Escobar, et al., J. Immunol., 144: 3449-3457, 1990. The release of histamine is determined by enzyme immunoassay. Measurements of release of peptide leukotrienes, prostaglandins, and TNFα are performed using enzyme immunoassays as described in Moriya, et al. A reduction in release of one or more of these mediators in cells treated with an inventive siRNA relative to the level of release in cells not treated with the siRNA indicates that the siRNA is effective in inhibiting mast cell responses and in reducing IgE-mediated responses and signs and symptoms of diseases or conditions associated with IgE-mediated hypersensitivity. Similar methods may be used to test shRNAs or RNAi vectors. The siRNAs, shRNAs, or RNAi vectors may be administered in combination with any of the delivery agents described above.

### Example 3

### Effect of Inventive siRNAs in a Murine Model

This example describes evaluation of the effect of administration of certain of the inventive siRNAs on various inflammatory responses in the lung in a typical murine model of allergic airway inflammation and hyperresponsiveness (Poynter, M., et al., Am. J. Path. 160(4): 1325-1334, 2002)

Six week old female BALB/c mice are purchased from the Jackson Laboratories (Bar Harbor, Me.) and are housed and maintained under standard conditions. Mice are divided into a number of groups, each of which is given an siRNA composition according to a different protocol as described below. Mice in each group are administered OVA (20 μg, grade V ovalbumin, Sigma, St. Louis, Mo.) with Alum (2.25 mg, Imject Alum, Pierce, Rockford, Ill.) via intraperitoneal injection on days 0 and 14 and are challenged with aerosolized OVA at days 21, 22, and 23, as previously described (Cieslewicz, G., et al., J. Clin. Invest., 104:301-308, 1999; Takeda, T., et al., J. Exp. Med., 186:449-454, 1997). Mice are euthanized by a lethal dose of pentobarbital via intraperitoneal injection.

siRNA compositions comprising siRNAs targeted to the FCεRIα chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2 either alone or in combination are administered to separate groups of mice at a variety of different times in relation to antigen challenge. For example, some groups are administered siRNA compositions weeks, days, or hours prior to antigen challenge. Some groups are administered siRNA compositions on days 21, 22, and/or 23. Some groups are administered siRNA compositions following antigen challenge. Certain groups are given a single dose of siRNA by any of a variety of different routes (inhalational, intravenous, etc.) while others are given a series of treatments separated by various time intervals. A range of doses is used. Comparison of the efficacy of these various treatment schemes allows selection of the optimum regimen.

In general, siRNAs may be delivered using any available route including oral or intravenous. However, because allergic rhinitis and asthma involve responses by cells in the nasal passages, upper airways, and the lung, a focus is on methods that deliver siRNAs into cells in the respiratory tract. Many different methods have been used to deliver small molecule drugs, proteins, and DNA/polymer complexes into the upper airways and/or lungs of mice, including instillation, aerosol (both liquid and dry-powder) inhalation, intratracheal administration, and intravenous injection. By instillation, mice are usually lightly anesthetized and held vertically upright. Therapeutics (i.e., siRNAs or siRNA/polymer complexes as described below) in a small volume (e.g., 30-50 μl) are applied slowly to one nostril where the fluid is inhaled (Densmore, C. L., et al., Mol. Therapy. 1:180-188, 1999). The animals are maintained in the upright position for a short period of time to allow instilled fluid to reach the lungs (Arppe, J., et al., Intl. J. Pharm. 161:205-214, 1998). Instillation is effective to deliver therapeutics to both the upper airways and the lungs and can be repeated multiple times on the same mouse.

By aerosol, liquid and dry-powder are usually applied differently. Liquid aerosols are produced by a nebulizer into a sealed plastic cage, where the mice are placed (Densmore, et al.). Because aerosols are inhaled as animals breathe, the method can be inefficient and imprecise. Dry-powder aerosols are usually administered by forced ventilation on anesthetized mice. This method can be very effective as long as the aerosol particles are large and porous (see below) (Edwards, D. A., et al., Science 276:1868-1871, 1997). For intratracheal administration, a solution containing therapeutics is injected via a tube into the lungs of anesthetized mice (Griesenbach, U., et al., Gene Ther. 5:181-188, 1998). Although it is quite efficient for delivery into the lungs, it misses the upper airways. Intravenous injection of a small amount of DNA (˜1 μg) in complexes with protein and polyethyleneimine has been shown to transfect endothelial cells and cells in interstitial tissues of the lung (Orson, F. M., et al., Gene Therapy 9:463-471, 2002).

Airway inflammation is assessed by performing broncheoalveolar lavage (BAL) 48 hours following antigen challenge and determining the number of inflammatory cells present in the lavage. Briefly, BAL is collected immediately on euthanization by instillation and recovery of 800 μl of 0.9% NaCl. Total cells in BAL are counted and 2×10⁴ cells are centrifuged onto glass slides at 800 rpm. Cytospins are stained using the Hema32 kit (Biochemical Sciences, Inc., Swedesboro, N.J.), and differential cell counts are performed on 500 cells. The number of macrophages, eosinophils, neutrophils, and lymphocytes in BAL from mice treated with the different siRNAs and according to the various treatment protocols are compared both between different groups and with controls that received either no siRNA (vehicle only) or an unrelated siRNA. In some animals, rather than performing BAL, lungs are removed, washed with PBS, fixed in 10% formalin, and stained with H&E. Cell counts are performed visually. A lower number of macrophages, eosinophils, neutrophils, and/or lymphocytes in BAL or in lung sections from mice that are treated with an inventive siRNA relative to the number in mice that are not treated indicates that the siRNA is effective. A lesser accumulation of mucus and the presence of low cuboidal cells in mice that are treated with an inventive siRNA rather than abundant intracytoplasmic accumulation of mucus and the presence of hyperplastic columnar epithelial cells as seen in mice that receive no siRNA or an unrelated siRNA indicates that the siRNA is effective in reducing IgE-mediated responses and signs and symptoms of diseases or conditions associated with IgE-mediated hypersensitivity.

More chronic responses are assessed using an improved murine model of chronic asthmatic inflammation described in Temelkovski, et al., referenced above, and Foster, P. S., et al., Lab Invest., 82(4): 455-462, 2002, in which sensitized mice are subjected to chronic inhalational challenge with low levels of OVA. In some groups of mice subjected to this protocol siRNA treatment is performed at intervals during the period of chronic inhalational challenge while in other groups siRNAs treatment is only performed prior to or up to several days following the initial antigen challenge. Indicators of chronic inflammation such as subepithelial fibrosis, hypertrophy of the tracheal epithelium, and mucus cell hyperplasia/metaplasia in the pulmonary airways are assessed as described in Foster, et al. A lesser degree of subepithelial fibrosis, hypertrophy of the tracheal epithelium, and mucus cell hyperplasia/metaplasia in the pulmonary airways in mice treated with an inventive siRNA relative to the level observed in mice that receive no siRNA or an unrelated siRNA indicates that the siRNA is effective in reducing IgE-mediated responses and signs and symptoms of diseases or conditions associated with IgE-mediated hypersensitivity.

Serum IgE specific for OVA is measured using standard techniques. A lower level of OVA-specific serum IgE in mice that are treated with an inventive siRNA relative to the level of OVA-specific serum IgE in mice that receive no siRNA or an unrelated siRNA indicates that the siRNA is effective in reducing IgE-mediated responses and signs and symptoms of diseases or conditions associated with IgE-mediated hypersensitivity.

Pulmonary function is assessed as follows. Mice are anesthetized with pentobarbital. Tracheotomized mice from each group are mechanically ventilated for the assessment of pulmonary function as described in Irvin, C. G., et al., Am. J. Physiol., 272:L1053-1058, 1997. Pressure, flow, and volume are used to calculate pulmonary resistance after challenge with inhaled doses of aerosolized methacholine as previously described (Takeda, et al.). A lower value for pulmonary resistance in mice that are treated with an inventive siRNA relative to pulmonary resistance in mice that are not treated indicates that the siRNA is effective in reducing IgE-mediated responses and signs and symptoms of diseases or conditions associated with IgE-mediated hypersensitivity. Alternatively, airway response is assessed by measuring methacholine-induced airflow obstruction in awake mice placed in a whole-body plethysmograph as described in Hansen, G., et al., J. Clin. Invest., 103: 175-183, 1999. Similar methods may be used to test shRNAs or RNAi vectors. The siRNAs, shRNAs, or RNAi vectors may be administered in combination with any of the delivery agents described above.

### Example 4

### Evaluation of Delivery Agents that Facilitate Cellular Uptake of RNAi Agents

This example describes testing a variety of delivery agents for their ability to enhance cellular uptake of RNAi agents.

Cationic polymers. The ability of cationic polymers to promote intracellular uptake of DNA is believed to result partly from their ability to bind to DNA and condense large plasmid DNA molecules into smaller DNA/polymer complexes for more efficient endocytosis. siRNA duplexes are only approximately 21 nucleotides in length, suggesting that they probably cannot be condensed much further. However, the ability of cationic polymers to bind negatively charge siRNA and interact with the negatively charged cell surface may facilitate intracellular uptake of siRNAs. Thus, the capacity of known cationic polymers in siRNA transfection including, but not limited to, imidazole group-modified PLL (17), polyethyleneimine (PEI) (22), Polyvinylpyrrolidone (PVP) (23), and chitosan (24, 25). will be investigated.

In addition, novel cationic polymers and oligomers developed in Robert Langer's laboratory will be investigated. Efficient strategies to synthesize and test large libraries of novel cationic polymers and oligomers from diacrylate and amine monomers in DNA transfection have been developed by Langer and coworkers. These polymers are referred to herein as poly(β-amino ester) (PAE) polymers. In their first “proof-of-principle” study, they synthesized a library of 140 polymers from 7 diacrylate monomers and 20 amine monomers (19). Of the 140 members, 70 were found sufficiently water-soluble (2 mg/ml, 25 mM acetate buffer, pH=5.0). Fifty-six of the 70 water-soluble polymers interacted with DNA as shown by electrophoretic mobility shift. Most importantly, they found two of the 56 polymers mediated DNA transfection into COS-7 cells. Transfection efficiencies of the novel polymers were 4-8 times higher than PEI and equal or better than Lipofectamine 2000.

Since the initial study, the Langer group has constructed and screened a library of 2,400 cationic polymers, and obtained another 40 or so polymers that promote efficient DNA transfection (D. Anderson and R. Langer, personal communication). Because structural variations could have a significant impact on DNA binding and transfection efficacies (18), it is preferable to test many polymers for their ability to promote intracellular uptake of siRNA. Furthermore, it is possible that in the transition to an in vivo system, certain polymers will likely be excluded as a result of studies of their in vivo performance, absorption, distribution, metabolism, and excretion (ADME). Thus in vivo testing is important.

Together, at least approximately 50 cationic polymers will be tested in siRNA transfection experiments. Most of them will be PAE and imidazole group-modified PLL as described above. PEI, PVP, and chitosan will be purchased from commercial sources. To screen these polymers rapidly and efficiently, the library of PAE polymers that successfully transfects cells has already been moved into solution into a 96-well plate. Storage of the polymers in this standard 96 well format allows for the straightforward development of a semi-automated screen, using a sterile Labcyte EDR 384S/96S micropipettor robot. The amount of polymer will be titrated (using a predetermined amount of siRNA) to define proper polymer siRNA ratios and the most efficient delivery conditions. Depending on the specific assay, the semi-automated screen will be slightly different as described below.

Characterization of siRNA/polymer complexes. For various cationic polymers to facilitate intracellular uptake of siRNA, they should be able to form complexes with siRNA. This issue will be examined this by electrophoretic mobility shift assay (EMSA) following a similar protocol to that described in (19). Briefly, siRNAs targeted to transcripts encoding any of the proteins discuss above (the FCcRIα chain, the FCεRIε chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2) will be mixed with each of the approximately 50 or polymers at the ratios of 1:0.1, 1:0.3, 1:0.9, 1:2.7, 1:8.1, and 1:24.3 (siRNA/polymer, w/w) in 96-well plates using micropipettor robot. The mixtures will be loaded into 4% agarose gel slab capable of assaying up to 500 samples using a multichannel pipettor. Migration patterns of siRNA will be visualized by ethidium bromide staining. If the mobility of an siRNA is reduced in the presence of a polymer, the siRNA forms complexes with that polymer. Based on the ratios of siRNA to polymer, it may be possible to identify the neutralizing ratio. It is expected that the various cationic polymers will form complexes with siRNA because they have been shown to form complexes with DNA. Those polymers that do not bind siRNA will be less preferred and further examination will focus on those polymers that do bind siRNA.

Cytotoxicity of imidazole group-modified PLL, PEI, PVP, chitosan, and some PAE polymers has been measured alone or in complexes with DNA in cell lines. Because cytotoxicity changes depending on bound molecules, the cytotoxicity of various polymers in complexes with siRNA will be measured in a variety of cells, e.g., epithelial cell lines, mast cell lines, T cell lines, DC cell lines. Suitable cell lines include, e.g., MDCK cells. Briefly, siRNAs will be mixed with different amounts of polymers as above, using the sterile Labcyte micropipettor robot. The complexes will be applied to epithelial cells in 96-well plates for 4 hrs. Then, the polymer-containing medium will be replaced with normal growth medium. 24 hrs later, the metabolic activity of the cells will be measured in the 96-well format using the MTT assay (26). It is expected that cytotoxicity of siRNA/polymer complexes will be similar to that of DNA/polymer complexes. Those polymers that kill 90% or more cells at the lowest amount used will be less preferred, and the focus of further investigation will be polymers that do not kill more than 90% of the cells at the lowest amount used.

siRNA uptake by cultured cells. Once siRNA/polymer complexes have been characterized, their ability to promote cellular uptake of siRNA will be tested, starting with cultured cells using two different assay systems. In the first approach, the effect of a GFP-specific siRNA referred to herein as GFP-949 (sense: 5′-GGCUACGUCCAGGAGCGCAUU-3′ (SEQ ID NO: 320); antisense: 5′-UGCGCUCCUGGACGUAGCCUU-3′ (SEQ ID NO: 321) on GFP expression in GFP-expressing cells is measured, because a decrease in GFP expression is easily quantified by measuring fluorescent intensity. Briefly, GFP-949/polymer at the same ratios as above will be applied to cells in 96-well plates. A variety of different cell types may be used. For convenience, a well characterized cell line such as MDCK cells may be used. Other suitable cells include mast cell lines, dendritic cell lines, etc. As negative controls, either no siRNA or an siRNA unrelated in sequence to any of the test siRNAs will be used. As a positive control, GFP-949 will be introduced into cells by electroporation. Thirty-six hrs later, cells will be lysed in 96-well plates and fluorescent intensity of the lysates measured by a fluorescent plate reader. The capacities of various polymers to promote cellular uptake of siRNA will be indicated by the overall decrease in GFP intensity. Alternatively, cells will be analyzed for GFP expression using a flow cytometer that is equipped to handle samples in the 96-well format. The capacities of various polymers to promote cellular uptake of siRNA will be indicated by the percentage of cells with reduced GFP intensity and the extent of decrease in GFP intensity. Results from these assays will also shed light on the optimal siRNA:polymer ratio for most efficient transfection.

In the second approach, inhibition of mast cell activity will be measured directly. As described above, siRNA/polymer (e.g., siRNAs targteted to the FCεRIα chain, the FCεRIβ chain, c-Kit, Lyn, Syk, etc.) at various ratios will be applied to mast cells in 96-well plates. As a positive control, siRNA will be introduced into mast cells by transfection or electroporation. As negative controls, an unrelated siRNA such as GFP-949 or no siRNA will be used.

If the release of mediators is substantially lower in mast cell cultures that are treated with siRNA/polymer than those that are not treated, it will be concluded that the polymer promotes siRNA transfection. By comparing mediator release in cultures in which siRNA is introduced by transfection or electroporation, the relative transfection efficiency of siRNAs and siRNA/polymer compositions will be estimated.

The most effective cationic polymers from the initial two screens will be verified in the virus infection assay in 96-well plates by titrating both siRNA and polymers. Based on the results obtained, the capacity of a number of the most effective polymers at the most effective siRNA:polymer ratios will be further analyzed in MDCK cells and/or mast cells in 24-well plates and 6-well plates. A number of the most effective polymers will be selected for further studies in mice as described in Example 5.

Other delivery agents. As an alternative cationic polymers for efficient promotion of intracellular uptake of siRNA in cultured cells, arginine-rich peptides will be investigated in siRNA transfection experiments. Because ARPs are thought to directly penetrate the plasma membrane by interacting with the negatively charged phospholipids (33), whereas most currently used cationic polymers are thought to promote cellular uptake of DNA by endocytosis, the efficacy of ARPs in promoting intracellular uptake of siRNA will be investigated. Like cationic polymers, ARPs and polyarginine (PLA) are also positively charged and likely capable of binding siRNA, suggesting that it is probably not necessary to covalently link siRNA to ARPs or PLAs. Therefore, ARPs or PLAs will be treated similarly to other cationic polymers. The ability of the ARP from Tat and different length of PLAs (available from Sigma) to promote cellular uptake of siRNA will be determined as described above.

### Example 5

### Testing of siRNAs and siRNA/Carrier Compositions in Mice

The ability of identified polymers to promote siRNA uptake by cells in the respiratory tract in mice is evaluated as described in U.S. Ser. No. 10/674,159, and the efficacies of siRNA/carrier compositions (siRNA/polymer compositions, siRNA/cationic polymer compositions, siRNA/arginine-rich peptide compositions, etc.) in preventing and treating allergic and asthmatic signs and symptoms in mice is examined as described in Example 3. Demonstration of siRNA inhibition of such signs and/or symptoms in mice will provide evidence for their potential use in humans to prevent or treat allergic rhinitis and/or asthma, e.g., by intranasal or pulmonary administration of siRNAs. Similar methods may be used to test shRNAs or RNAi vectors. The siRNAs, shRNAs, or RNAi vectors may be administered in combination with any of the delivery agents described above.

### Example 6

### Effect of Inventive shRNAs Transcribed from DNA Vectors

Effective siRNA therapy depends on the ability to deliver a sufficient amount of siRNA into appropriate cells in vivo. As an alternative to the approaches described above, DNA vectors from which siRNA precursors can be transcribed and processed into effective siRNAs may be used.

We have previously shown that RNAi agents transcribed from a DNA vector can inhibit CD8α expression to the same extent as synthetic siRNA introduced into the same cells. Specifically, we found that one of the five siRNAs designed to target the CD8a gene, referred to as CD8-61, inhibited CD8 but not CD4 expression in a mouse CD8⁺CD4⁺T cell line (12). By testing various hairpin derivatives of CD8-61 siRNA, we found that CD8-61F had a similar inhibitory activity as CD8-61 (44). CD8-61F was constructed into pSLOOP III, a DNA vector in which transcription is driven by the H1 RNA promoter, resulting in the plasmid pSLOOP III-CD8-61F. The H1 RNA promoter is compact (45) and transcribed by polymerase III (pol III). The Pol III promoter was used because it normally transcribes small RNA and has been used to generate siRNA-type silencing previously (46). To test the DNA vector, we used HeLa cells that had been transfected with a CD8a expressing vector. Transient transfection of the pSLOOP III-CD8-61F plasmid into CD8α-expressing HeLa cells resulted in reduction of CD8a expression to the same extent as HeLa cells that were transfected with synthetic CD8-61 siRNA. In contrast, transfection of a promoter-less vector did not significantly reduce CD8α expression. These results show that an RNA hairpin can be transcribed from a DNA vector and then processed into siRNA for RNA silencing. A similar approach may be used to design DNA vectors that express siRNA precursors specific for the transcripts described herein, e.g., transcripts encoding the FCεRIα chain, the FCεRIβ chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2.

Studies of siRNA generated from shRNA transcribed from DNA vectors in cultured cells and animal models. To express siRNA precursors from a DNA vector, hairpin derivatives of siRNA (specific for a target transcript) that can be processed into siRNA duplexes will be identified. For example, FIG. 7A shows schematic diagrams of HFcεRα-338 and GFP-949 siRNA and their hairpin derivatives/precursors. Similar hairpin derivatives/precursors may be constructed for any of the inventive siRNAs described herein. (Note that an shRNA may be referred to as a “derivative” of the corresponding siRNA because the design of an shRNA may be based upon, i.e., derived from, that of a corresponding siRNA, i.e., an siRNA with the same or a substantially identical duplex portion. However, within the cell an shRNA serves as a “precursor” of the corresponding siRNA, i.e., the hairpin is processed to generate the corresponding siRNA. Thus as will be evident to one of ordinary skill in the art, the terms may be used interchangeably or alternately, depending upon the context.) FIG. 7B shows hairpin tandem arrays of HFcεRα-338 and GFP-949H in two different orders. Similar hairpin tandem arrays may be constructed for any of the inventive siRNAs described herein, i.e., any two of the inventive siRNAs may be incorporated into a single hairpin tandem array.

FIG. 7C shows pSLOOP III expression vectors. Hairpin derivatives/precursors of siRNA are cloned into pSLOOP III vector alone (top), in tandem arrays (middle), or simultaneously with independent promoter and termination sequence (bottom). In addition, vectors from which two or more siRNA precursors can be transcribed will be produced. The same general approach described in U.S. patent application Ser. No. 10/674,159 will be employed, except that rather than testing siRNA hairpin derivatives for their ability to inhibit influenza virus production, siRNA hairpin derivatives will be tested for their ability to inhibit mast cell response (e.g., mediator release), T cell response, IgE production, and/or signs and symptoms of IgE-mediated hypersensitivity in mice or other animal models.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

### REFERENCES

1. Vaucheret, H., C. Beclin, and M. Fagard. 2001. Post-transcriptional gene silencing in plants. J. Cell Sci. 114:3083-3091.
2. Sharp, P. A. 2001. RNA interference-2001. Genes Dev. 15:485-490.
3. Brantl, S. 2002. Antisense-RNA regulation and RNA interference. Biochem. Biophy. Acta 1575:15-25.
4. Baulcombe, D. 2002. RNA silencing. Curr. Biol. 12:R82-R84.
5. Fire, A., S. Xu, M. K. Montgomery, S. A. Kostas, S. E. Driver, and M. C. C. 1998. Potent and specific genetic interference by double-stranded RNA in Caenorhabditis elegans. Nature 391:806-811.
6. Elbashir, S., J. Harborth, W. Lendeckel, A. Yalcin, K. Weber, and T. Tuschl. 2001. Duplexes of 21-nucleotide RNAs mediate RNA interference in cultured mammalian cells. Nature 411:494-498.
7. McManus, M. T., and P. A. Sharp. 2002. Gene silencing in mammals by short interfering RNAs. Nature Rev. Gene. 3:737-747.
8. Kumar, M., and G. G. Carmichael. 1998. Antisense RNA: function and fate of duplex RNA in cells of higher eukaryotes. Microbiol. Mol. Biol. Rev. 62:1415-1434.
9. Gitlin, L., S. Karelsky, and R. Andino. 2002. Short interfering RNA confers intracellular antiviral immunity in human cells. Nature 418:430-434.
10. Pderoso de Lima, M. C., S. Simoes, P. Pires, H. Faneca, and N. Duzgunes. 2001. Cationic lipid-DNA complexes in gene delivery: from biophysics to biological applications. Adv. Drug Deliv. Rev. 47:277-294.
11. Holen, T., M. Amarzguioui, M. T. Wiiger, E. Babaie, and H. Prydz. 2002. Positional effects of short interfering RNAs targeting the human coagulation trigger tissue factor. Nucleic Acids Res. 30:1757-1766.
12. McManus, M. T., B. B. Haines, C. P. Dillon, C. E. Whitehurst, L. Van Parijs, J. Chen, and P. A. Sharp. 2002. siRNA-mediated gene silencing in T-lymphocytes. J. Immunol. Nov. 15; 169(10):5754-60.
13. Elbashir, S. M., J. Martinez, A. Patkaniowska, W. Lendeckel, and T. Tuschl. 2001. Functional anatomy of siRNAs for mediating efficient RNAi in Drosophila melanogaster embryo lysate. EMBO J. 20:6877-6888.
14. Yang, D., H. Lu, and J. W. Erickson. 2000. Evidence that processed small dsRNAs may mediate sequence-specific mRNA degradation during RNAi in Drosophila embryos. Curr. Biol. 10:1191-1200.
15. Caplen, N. J., J. Fleenor, A. Fire, and R. A. Morgan. 2000. dsRNA-mediated gene silencing in cultured Drosophila cells: a tissue culture model for the analysis of RAN interference. Gene 252:95-105.
16. Edwards, D. A., J. Hanes, G. Caponetti, J. Hrkach, A. Ben-Jebria, M. L. Eskew, J. Mintzes, D. Deaver, N. Lotan, and R. Langer. 1997. Large porous particles for pulmonary drug delivery. Science 276:1868-1871.
17. Putnam, D., C. A. Gentry, D. W. Pack, and R. Langer. 2000. Polymer-based gene delivery with low cytotoxicity by a unique balance of side-chain termini. Proc. Natl. Acad. Sci. USA 98:1200-1205.
18. Lynn, D. M., and R. Langer. 2000. Degradable Poly(-amino esters): Synthesis, Characterization, and Self-Assembly with Plasmid DNA. J. Am. Chem. Soc. 122:10761-10768.
19. Lynn, D. M., D. G. Anderson, D. Putnam, and R. Langer. 2001. Accelerated discovery of synthetic transfection vectors: parallel synthesis and screening of a degrable polymer library. J. Am. Chem. Soc. 123:8155-8156.
20. Han, S.-O., R. I. Mahato, Y. K. Sung, and S. W. Kim. 2000. Development of Biomaterials for gene therapy. Mol. Therapy. 2:302-317.
21. Soane, R. J., M. Frier, A. C. Perkins, N. S. Jones, S. S. Davis, and L. Illum. 1999. Evaluation of the clearance characteristics of bioadhesive systems in humans. Int. J. Pharm. 178:55-65.
22. Boussif, O., F. Lezoualc'h, M. A. Zanta, M. D. Mergny, D. Scherman, B. Demeneix, and J. P. Behr. 1995. A versatile vector for gene and oligonucleotide transfer into cells in culture and in vivo: polyethylenimine. Proc. Natl. Acad. Sci. USA 92:7297-7301.
23. Astafieva, I., I. Maksimova, E. Lukanidin, V. Alakhov, and A. Kabanov. 1996. Enhancement of the polycation-mediated DNA uptake and cell transfection with Pluronic P85 block copolymer. FESB Lett. 389:278-280.
24. Davis, S. S. 1999. Delivery of peptide and non-peptide drugs through the respiratory tract. Pharm. Sci. Technol. Today 2:450-457.
25. Roy, K., H.-Q. Mao, S.-K. Huang, and K. W. Leong. 1999. Oral delivery with chitosan/DNA nanoparticles generates immunologic protection in murine model of peanut allergic rhinitis. Nat. Med. 5:387-391.
26. Hansen, M. B., S. E. Nielsen, and K. Berg. 1989. Re-examination and further development of a precise and rapid dye method for measuring cell growth/cell kill. J. Immunol. Methods 119:203-210.
27. Green, M., and P. M. Loewenstein. 1988. Autonomous functional domains of chemically synthesized human immunodeficiency virus tat trans-activator protein. Cell 55:1179-1188.
28. Frankel, A. D., and C. O. Pabo. 1988. Cellular uptake of the tat protein from human immunodeficiency virus. Cell 55:1189-1193.
29. Elliott, G., and P. O'Hare. 1997. Intercellular trafficking and protein delivery by a herpesvirus structural protein. Cell 88:223-233.
30. Joliot, A., C. Pernelle, H. Deagostini-Bazin, and A. Prochiantz. 1991. Antennapedia homeobox peptide regulates neural morphogenesis. Proc. Natl. Acad. Sci. USA 88:1864-1868.
31. Fawell, S., J. Seery, Y. Daikh, C. Moore, L. L. Chen, B. Pepinsky, and J. Barsoum. 1994. Tat-mediated delivery of heterologous proteins into cells. Proc. Natl. Acad. Sci. USA 91:664-668.
32. Schwarze, S. R., A. Ho, A. Vocero-Akbani, and S. F. Dowdy. 1999. In vivo protein transduction:delivery of a biologically active protein. Science 285:1569-1572.
33. Derossi, D., G. Chassaing, and A. Prochiantz. 1998. Trojan peptides: the penetratin system for intracellular delivery. Trends Cell Biol. 8:84-87.
34. Troy, C. M., D. Derossi, A. Prochiantz, L. A. Greene, and M. L. Shelanski. 1996. Downregulation of Cu/Zn superoxide dismutase leads to cell death via the nitric oxide-peroxynitrite pathway. J. Neurosci. 16:253-261.
35. Allinquant, B., P. Hantraye, P. Mailleux, K. Moya, C. Bouillot, and A. Prochiantz. 1995. Downregulation of amyloid precursor protein inhibits neurite outgrowth in vitro. J. Cell Biol. 128:919-927.
36. Futaki, S., T. Suzuki, W. Ohashi, T. Yagami, S. Tanaka, K. Ueda, and Y. Sugiura. 2001. Arginine-rich peptides. An abundant source of membrane-permeable peptides having potential as carriers for intracellular protein delivery. J. Biol. Chem. 276:5836-5840.
37. Densmore, C. L., F. M. Orson, B. Xu, B. M. Kinsey, J. C. Waldrep, P. Hua, B. Bhogal, and V. Knight. 1999. Aerosol delivery of robust polyethyleneimine-DNA complexes for gene therapy and genetic immunization. Mol. Therapy. 1:180-188.
38. Arppe, J., M. Widgred, and J. C. Waldrep. 1998. Pulmonary pharmacokinetics of cyclosporin A liposomes. Intl. J. Pharm. 161:205-214.
39. Griesenbach, U., A. chonn, R. Cassady, V. Hannam, C. Ackerley, M. Post, A. K. Transwell, K. Olek, H. O'Brodovich, and L.-C. Tsui. 1998. Comparison between intratracheal and intravenous administration of liposome-DNA complexes for cystic fibrosis lung gene therapy. Gene Ther. 5:181-188.
40. Orson, F. M., L. Song, A. Gautam, C. L. DEnsmore, B. Bhogal, and B. M. Kinsey. 2002. Gene delivery to the lung using protein/polyethyleneimine/plasmid complexes. Gene Therapy 9:463-471.
41. Gautam, A., C. L. DEnsmore, E. Golunski, B. Xu, and J. C. Waldrep. 2001. Transgene expression in mouse airway epithelium by aerosol gene therapy with PEI-DNA complexes. Mol. Therapy. 3:551-556.
42. Tabata, Y., and Y. Ikada. 1988. Effect of size and surface charge of polymer microspheres on their phagocytosis by macrophage. J. Biomed. Mater. Res. 22:837-842.
43. Vanbever, R., J. D. Mintzes, J. Wang, J. Nice, D. chen, R. Batycky, L. R., and D. A. Edwards. 1999. Formulation and physiclal characterization of large porous particles for inhalation. Pharmaceutical Res. 16:1735-1742.
44. McManus, M. T., C. P. Peterson, B. B. Haines, J. Chen, and P. A. Sharp. 2002. Gene silencing using micro-RNA designed hairpins. RNA 8:842-850.
45. Myslinski, E., J. C. Ame, A. Krol, and P. Carbon. 2001. An unusually compact external promoter for RNA polymerase III transcription of the human H1 RNA gene. Nucleic Acids Res. 29:2502-2509.
46. Brummelkamp, T. R., R. Bernards, and R. Agami. 2002. A system for stable expression of short interfering RNAs in mammalian cells. Science 296:550-553.
47. Paddison, P. J., A. A. Caudy, E. Bernstein, G. J. Hannon, and D. S. Conklin. 2002. Short hairpin RNAs (shRNAs) induce sequence-specific silencing in mammalian cells. Genes Dev. 16:948-958.
48. Gil, J., and M. Esteban. 2000. Induction of apoptosis by the dsRNA-dependent protein kinase (PKR): mechanism of action. Apoptosis 5:107-114.
49. Bitko, V., and S. Batik. 2001. Phenotypic silencing of cytoplasmic genes using sequence-specific double-stranded short interfering RNA and its application in the reverse genetic of wild type negative-strand RNA viruses. BMC Microbiol. 1:34-43.
50. Garcia-Sastre, A. (2002) Microbes & Inf. 4, 647-655.
51. Katze, M. G., He, Y. & Gale Jr., M. (2002) Nature Rev. Immunol. 2, 675-687.
52. Diaz, M. O., Ziemin, S., Le Beau, M. M., Pitha, P., Smith, S. D., Chilcote, R. R. & Rowley, J. D. (1988) Proc. Natl. Acad. Sci. USA 85, 5259-5263.
53. Diaz, M. O., Pomykala, H. M., Bohlander, S. K., Maltepe, E., Malik, K., Brownstein, B. & Olopade, O. I. (1994) Genomics 22, 540-552.
54. Kim, M.-J., Latham, A. G. & Krug, R. M. (2002) Proc. Natl. Acad. Sci. USA 99, 10096-10101.
55. Medcalf, L., Poole, E., Elton, D. & Digard, P. (1999) J. Virol. 73, 7349-7356.
56. Shapiro, G. I. & Krug, R. M. (1988) J. Virol. 62, 2285-2290.
57. Beaton, A. R. & Krug, R. M. (1986) Proc. Natl. Acad. Sci. USA 83, 6282-6286.

1. An RNAi agent targeted to a target transcript that encodes a protein selected from the group consisting of: FCεR α chain, FCεR β chain, c-Kit, Lyn, Syk, ICOS, OX40L, CD40, CD80, CD86, Re1A, Re1B, 4-1BB ligand, TLR1, TLR2, TLR3, TLR4, TLR5, TLR6, TLR7, TLR8, TLR9, CD83, SLAM, common γ chain, and COX-2 involved in development, pathogenesis, or symptoms of an IgE-mediated disease or condition.

2. The RNAi agent of claim 1, wherein the disease is allergic rhinitis or asthma.

3. (canceled)

4. The RNAi agent of claim 1, wherein the RNAi agent is an RNAi vector.

5. The RNAi agent of claim 1, wherein the RNAi agent is an siRNA or shRNA.

6. The RNAi agent of claim 5, wherein the siRNA or shRNA comprises a duplex portion at least 15 nucleotides long.

7. The RNAi agent of claim 5, wherein the siRNA or shRNA comprises a duplex portion approximately 19 nucleotides long.

8. The RNAi agent of claim 5, wherein the siRNA or shRNA comprises an antisense strand comprising a portion whose sequence is substantially complementary to a sequence listed in Tables 1-26 over at least 15 continuous nucleotides.

9. The RNAi agent of claim 5, wherein the siRNA or shRNA comprises an antisense strand comprising a portion whose sequence is 100% complementary to a sequence listed in Tables 1-26 over at least 15 continuous nucleotides.

10. The RNAi agent of claim 5, wherein the siRNA or shRNA comprises a sense strand comprising a portion whose sequence is substantially identical to a sequence listed in any of SEQ ID NOs: 1-315 over at least 15 continuous nucleotides.

11. The RNAi agent of claim 5, wherein the siRNA or shRNA comprises a sense strand comprising a portion whose sequence is 100% identical to a sequence listed in any of SEQ ID NOs: 1-315 over at least 15 continuous nucleotides.

12. A composition comprising the RNAi agent of claim 1.

13. The composition of claim 12, formulated as a dry powder.

14. A method of treating or preventing a disease or condition comprising the steps of:
(a) providing a subject at risk of or suffering from a disease or condition; and
(b) administering the RNAi agent of claim 1, to the subject.

15. The method of claim 14, wherein the IgE-mediated condition is allergic rhinitis or asthma.

16. (canceled)

17. The method of claim 14, wherein the composition is administered directly to the respiratory system of a subject by inhalational delivery.

18. The composition of claim 12, further comprising a pharmaceutically acceptable carrier.

19. The composition of claim 12, further comprising a delivery agent.

20. The method of claim 14, wherein the step of providing comprises providing a subject at risk of or suffering from sepsis, shock, or a burn-related injury, and wherein the step of administering comprises administering to the subject a composition comprising an RNAi agent targeted to a Toll-like receptor.

21-116. (canceled)

117. The composition of claim 12, formulated for aerosol delivery.

118. The method of claim 14, wherein the step of providing comprises providing a subject at risk of or suffering from an IgE-mediated condition.


SEQUENCE LISTING (326 sequences)

SEQ ID NO: 1 - RNA, 19 nt, Artificial
  Mouse sequences
uuggucauugugagugcca

SEQ ID NO: 2 - RNA, 19 nt, Artificial
  Mouse sequences
caagacaguggaaaauaca

SEQ ID NO: 3 - RNA, 19 nt, Artificial
  Mouse sequences
augucagaagcaaggauug

SEQ ID NO: 4 - RNA, 19 nt, Artificial
  Mouse sequences
uccuuugacaucagaugcc

SEQ ID NO: 5 - RNA, 19 nt, Artificial
  Mouse sequences
gcaaggugaucuacuacag

SEQ ID NO: 6 - RNA, 19 nt, Artificial
  Mouse sequences
gauucuguuugcuguggac

SEQ ID NO: 7 - RNA, 19 nt, Artificial
  Mouse sequences
gagauucagaagacuggaa

SEQ ID NO: 8 - RNA, 19 nt, Artificial
  Mouse sequences
caggaauugcauaaaugcu

SEQ ID NO: 9 - RNA, 19 nt, Artificial
  Human sequences
gaauauugugaaugccaaa

SEQ ID NO: 10 - RNA, 19 nt, Artificial
  Human sequences
gaagacaguggagaauaca

SEQ ID NO: 11 - RNA, 19 nt, Artificial
  Human Sequences
augucagcaccaacaaguu

SEQ ID NO: 12 - RNA, 19 nt, Artificial
  Human Sequences
ucuuccucaggugccaugg

SEQ ID NO: 13 - RNA, 19 nt, Artificial
  Human Sequences
cugggauguguacaaggug

SEQ ID NO: 14 - RNA, 19 nt, Artificial
  Human Sequences
gauucuguuugcuguggac

SEQ ID NO: 15 - RNA, 19 nt, Artificial
  Human Sequences
aaccaggaaaggcuucaga

SEQ ID NO: 16 - RNA, 19 nt, Artificial
  Human
cgucugugcucaaggauuu

SEQ ID NO: 17 - RNA, 19 nt, Artificial
  Mouse sequences
gauaugccuuuguuuugga

SEQ ID NO: 18 - RNA, 19 nt, Artificial
  Mouse sequences
uuacagugaguuggaagac

SEQ ID NO: 19 - RNA, 19 nt, Artificial
  Human sequences
gauaugccuuuguuuugga

SEQ ID NO: 20 - RNA, 19 nt, Artificial
  Human sequences
uuacagugaguuggaagac

SEQ ID NO: 21 - RNA, 19 nt, Artificial
  Mouse sequences
guuuguuagagauccugcc

SEQ ID NO: 22 - RNA, 19 nt, Artificial
  Mouse sequences
gauucuggaguguucaugu

SEQ ID NO: 23 - RNA, 19 nt, Artificial
  Mouse sequences
ggaucagcaaaugucacaa

SEQ ID NO: 24 - RNA, 19 nt, Artificial
  Mouse sequences
caaaaccagaaauccugac

SEQ ID NO: 25 - RNA, 19 nt, Artificial
  Mouse sequences
caacgaugugggcaagagu

SEQ ID NO: 26 - RNA, 19 nt, Artificial
  Mouse sequences
cgaggagauaaauggaaac

SEQ ID NO: 27 - RNA, 19 nt, Artificial
  Mouse sequences
caacuuccuuaugaucaca

SEQ ID NO: 28 - RNA, 19 nt, Artificial
  Mouse sequences
ugggaguuucccagaaaca

SEQ ID NO: 29 - RNA, 19 nt, Artificial
  Mouse sequences
cccuggucauuacagaaua

SEQ ID NO: 30 - RNA, 19 nt, Artificial
  Mouse sequences
guugcuauggugaucuuuu

SEQ ID NO: 31 - RNA, 19 nt, Artificial
  Mouse sequences
uccucgccuccaagaauug

SEQ ID NO: 32 - RNA, 19 nt, Artificial
  Mouse sequences
uucacagagacuuggcagc

SEQ ID NO: 33 - RNA, 19 nt, Artificial
  Mouse sequences
cggaucacaaagauuugcg

SEQ ID NO: 34 - RNA, 19 nt, Artificial
  Mouse sequences
cuagccagagacaucagga

SEQ ID NO: 35 - RNA, 19 nt, Artificial
  Mouse sequences
gugaaguggauggcaccag

SEQ ID NO: 36 - RNA, 19 nt, Artificial
  Mouse sequences
gucugguccuaugggauuu

SEQ ID NO: 37 - RNA, 19 nt, Artificial
  Mouse sequences
ucaaggaaggcuuccggau

SEQ ID NO: 38 - RNA, 19 nt, Artificial
  Mouse sequences
ucaugaagacuugcuggga

SEQ ID NO: 39 - RNA, 19 nt, Artificial
  Mouse sequences
uucagguauguugccuuua

SEQ ID NO: 40 - RNA, 19 nt, Artificial
  Mouse sequences
acuguugacaguucugaag

SEQ ID NO: 41 - RNA, 19 nt, Artificial
  Human sequences
guuuguuagagauccugcc

SEQ ID NO: 42 - RNA, 19 nt, Artificial
  Human sequences
ggugacuucaauuaugaac

SEQ ID NO: 43 - RNA, 19 nt, Artificial
  Human sequences
gauucuggaguguucaugu

SEQ ID NO: 44 - RNA, 19 nt, Artificial
  Human sequences
ggaucagcaaaugucacaa

SEQ ID NO: 45 - RNA, 19 nt, Artificial
  Human sequences
caaaaccagaaauccugac

SEQ ID NO: 46 - RNA, 19 nt, Artificial
  Human sequences
uacaacgaugugggcaaga

SEQ ID NO: 47 - RNA, 19 nt, Artificial
  Human sequences
gaggagauaaauggaaaca

SEQ ID NO: 48 - RNA, 19 nt, Artificial
  Human sequences
caacuuccuuaugaucaca

SEQ ID NO: 49 - RNA, 19 nt, Artificial
  Human sequences
aaugggaguuucccagaaa

SEQ ID NO: 50 - RNA, 19 nt, Artificial
  Human sequences
cccuggucauuacagaaua

SEQ ID NO: 51 - RNA, 19 nt, Artificial
  Human sequences
uuguugcuauggugaucuu

SEQ ID NO: 52 - RNA, 19 nt, Artificial
  Human sequences
uuccucgccuccaagaauu

SEQ ID NO: 53 - RNA, 19 nt, Artificial
  Human sequences
uucacagagacuuggcagc

SEQ ID NO: 54 - RNA, 19 nt, Artificial
  Human sequences
cggaucacaaagauuugug

SEQ ID NO: 55 - RNA, 19 nt, Artificial
  Human sequences
cuagccagagacaucaaga

SEQ ID NO: 56 - RNA, 19 nt, Artificial
  Human sequences
ugugaaguggauggcaccu

SEQ ID NO: 57 - RNA, 19 nt, Artificial
  Human sequences
gucugguccuaugggauuu

SEQ ID NO: 58 - RNA, 19 nt, Artificial
  Human sequences
aucaaggaaggcuuccgga

SEQ ID NO: 59 - RNA, 19 nt, Artificial
  Human sequences
uaaugaagacuugcuggga

SEQ ID NO: 60 - RNA, 19 nt, Artificial
  Human sequences
gauucagguauguugccuu

SEQ ID NO: 61 - RNA, 19 nt, Artificial
  Human sequences
uguugacaguucugaagaa

SEQ ID NO: 62 - RNA, 19 nt, Artificial
  Mouse sequences
gaagacucaaccaguacgu

SEQ ID NO: 63 - RNA, 19 nt, Artificial
  Mouse sequences
cuauuuaugugagagaucc

SEQ ID NO: 64 - RNA, 19 nt, Artificial
  Mouse sequences
guccaauaaacagcaaagg

SEQ ID NO: 65 - RNA, 19 nt, Artificial
  Mouse sequences
aagauccagaggaacaagg

SEQ ID NO: 66 - RNA, 19 nt, Artificial
  Mouse sequences
gcacuacaaaauuagaagu

SEQ ID NO: 67 - RNA, 19 nt, Artificial
  Mouse sequences
cagaagccaugggauaaag

SEQ ID NO: 68 - RNA, 19 nt, Artificial
  Mouse sequence
gucuggauggguuacuaua

SEQ ID NO: 69 - RNA, 19 nt, Artificial
  Mouse sequences
gccaaccucaugaagaccu

SEQ ID NO: 70 - RNA, 19 nt, Artificial
  Mouse sequences
uaguuugcuggauuuccuc

SEQ ID NO: 71 - RNA, 19 nt, Artificial
  Mouse sequences
uacaucgagcggaagaacu

SEQ ID NO: 72 - RNA, 19 nt, Artificial
  Mouse sequences
guacacagcaagggaaggu

SEQ ID NO: 73 - RNA, 19 nt, Artificial
  Mouse sequences
gauugucaccuaugggaag

SEQ ID NO: 74 - RNA, 19 nt, Artificial
  Mouse sequence
uucccuacccagggagaac

SEQ ID NO: 75 - RNA, 19 nt, Artificial
  Mouse sequences
uggagaacugcccagauga

SEQ ID NO: 76 - RNA, 19 nt, Artificial
  Human sequences
gaagacucaaccaguacgu

SEQ ID NO: 77 - RNA, 19 nt, Artificial
  Human sequences
cuauuuaugugagagaucc

SEQ ID NO: 78 - RNA, 19 nt, Artificial
  Human sequences
uccaauaaacagcaaaggc

SEQ ID NO: 79 - RNA, 19 nt, Artificial
  Human sequences
aagauccagaggaacaagg

SEQ ID NO: 80 - RNA, 19 nt, Artificial
  Human sequences
gcacuacaaaauuagaagu

SEQ ID NO: 81 - RNA, 19 nt, Artificial
  Human sequences
cagaagccaugggauaaag

SEQ ID NO: 82 - RNA, 19 nt, Artificial
  Human sequences
gucuggauggguuacuaua

SEQ ID NO: 83 - RNA, 19 nt, Artificial
  Human sequences
aagccaaccucaugaagac

SEQ ID NO: 84 - RNA, 19 nt, Artificial
  Human sequences
caguuugcuggauuuccug

SEQ ID NO: 85 - RNA, 19 nt, Artificial
  Human sequences
uacaucgagcggaagaacu

SEQ ID NO: 86 - RNA, 19 nt, Artificial
  Human sequences
guacacagcaagggaaggu

SEQ ID NO: 87 - RNA, 19 nt, Artificial
  Human sequences
aauugucaccuaugggaaa

SEQ ID NO: 88 - RNA, 19 nt, Artificial
  Human sequences
aauucccuacccagggaga

SEQ ID NO: 89 - RNA, 19 nt, Artificial
  Human sequences
uggagaacugcccagauga

SEQ ID NO: 90 - RNA, 19 nt, Artificial
  Mouse sequences
aggaaggcacaccacuaca

SEQ ID NO: 91 - RNA, 19 nt, Artificial
  Mouse sequences
aagaagcccuucaaccggc

SEQ ID NO: 92 - RNA, 19 nt, Artificial
  Mouse sequences
aaccucaucagggaauaug

SEQ ID NO: 93 - RNA, 19 nt, Artificial
  Mouse sequences
auggacacagagguguacg

SEQ ID NO: 94 - RNA, 19 nt, Artificial
  Mouse sequences
ugaaaaccguggcugugaa

SEQ ID NO: 95 - RNA, 19 nt, Artificial
  Mouse sequences
uuugugcacagagaucugg

SEQ ID NO: 96 - RNA, 19 nt, Artificial
  Mouse sequences
cugcgugcugaugaaaacu

SEQ ID NO: 97 - RNA, 19 nt, Artificial
  Mouse sequences
gaaugcaucaacuacuaca

SEQ ID NO: 98 - RNA, 19 nt, Artificial
  Mouse sequences
aggaaggcacaccacuaca

SEQ ID NO: 99 - RNA, 19 nt, Artificial
  Human sequences
aagaagcccuucaaccggc

SEQ ID NO: 100 - RNA, 19 nt, Artificial
  Human sequences
aaccucaucagggaauaug

SEQ ID NO: 101 - RNA, 19 nt, Artificial
  Human sequences
auggacacagagguguacg

SEQ ID NO: 102 - RNA, 19 nt, Artificial
  Human sequences
ugaaaaccguggcugugaa

SEQ ID NO: 103 - RNA, 19 nt, Artificial
  Human sequences
uuugugcacagagaucugg

SEQ ID NO: 104 - RNA, 19 nt, Artificial
  Human sequences
cugcgugcugaugaaaacu

SEQ ID NO: 105 - RNA, 19 nt, Artificial
  Human sequences
gaaugcaucaacuacuaca

SEQ ID NO: 106 - RNA, 19 nt, Artificial
  Mouse sequences
uaacaggagaaaucaaugg

SEQ ID NO: 107 - RNA, 19 nt, Artificial
  Mouse sequences
uaacaggagaaaucaaugg

SEQ ID NO: 108 - RNA, 19 nt, Artificial
  Mouse sequences
agugaauacauguucaugg

SEQ ID NO: 109 - RNA, 19 nt, Artificial
  Mouse sequences
auucugcugguguuuuguu

SEQ ID NO: 110 - RNA, 19 nt, Artificial
  Mouse sequences
uauuuagccugaaagcugc

SEQ ID NO: 111 - RNA, 19 nt, Artificial
  Mouse sequences
uaacaggagaaaucaaugg

SEQ ID NO: 112 - RNA, 19 nt, Artificial
  Mouse sequences
ggugaauacauguucauga

SEQ ID NO: 113 - RNA, 19 nt, Artificial
  Mouse sequences
cuucugcugguguuuuguu

SEQ ID NO: 114 - RNA, 19 nt, Artificial
  Human sequence
cauuuagccugaaagcugc

SEQ ID NO: 115 - RNA, 19 nt, Artificial
  Mouse sequences
gaugagaaucuggaaaacg

SEQ ID NO: 116 - RNA, 19 nt, Artificial
  Mouse sequences
aaguggaagaagacgcuaa

SEQ ID NO: 117 - RNA, 19 nt, Artificial
  Mouse sequences
cuuccuucaaagaacuacc

SEQ ID NO: 118 - RNA, 19 nt, Artificial
  Mouse sequences
ugcaaagaaaaccaggaga

SEQ ID NO: 119 - RNA, 19 nt, Artificial
  Human sequences
aagauucgagaggaacaag

SEQ ID NO: 120 - RNA, 19 nt, Artificial
  Human sequences
guauccucgaauucaaagu

SEQ ID NO: 121 - RNA, 19 nt, Artificial
  Human sequences
uggugaauucuguguccuu

SEQ ID NO: 122 - RNA, 19 nt, Artificial
  Human sequences
uacuaggcaccuuugugag

SEQ ID NO: 123 - RNA, 19 nt, Artificial
  Mouse sequence
agaaucagacacugucugu

SEQ ID NO: 124 - RNA, 19 nt, Artificial
  Mouse sequences
aacagguaguggaaugaug

SEQ ID NO: 125 - RNA, 19 nt, Artificial
  Mouse sequences
auuccaaggcagguaagau

SEQ ID NO: 126 - RNA, 19 nt, Artificial
  Mouse sequences
uugucauuugaccuccaug

SEQ ID NO: 127 - RNA, 19 nt, Artificial
  Mouse sequences
ugugcucugugguaaugua

SEQ ID NO: 128 - RNA, 19 nt, Artificial
  Mouse sequences
cacaugugcacauauccua

SEQ ID NO: 129 - RNA, 19 nt, Artificial
  Human sequences
gcuguguaucuucauagaa

SEQ ID NO: 130 - RNA, 19 nt, Artificial
  Human sequences
acgauacagagaugcaaca

SEQ ID NO: 131 - RNA, 19 nt, Artificial
  Human sequences
uacucagagcugcaaauac

SEQ ID NO: 132 - RNA, 19 nt, Artificial
  Human sequences
uugaauugcaaccaggugc

SEQ ID NO: 133 - RNA, 19 nt, Artificial
  Human sequences
uugucaaugugacugaucc

SEQ ID NO: 134 - RNA, 19 nt, Artificial
  Mouse sequences
cuacaucucuguuucucga

SEQ ID NO: 135 - RNA, 19 nt, Artificial
  Mouse sequences
caaagcaucugaagcuaug

SEQ ID NO: 136 - RNA, 19 nt, Artificial
  Mouse sequences
cuugaugacugaaguggaa

SEQ ID NO: 137 - RNA, 19 nt, Artificial
  Mouse sequence
gcaacuugauaugucaugu

SEQ ID NO: 138 - RNA, 19 nt, Artificial
  Human sequences
ucuucuacgugagcaauug

SEQ ID NO: 139 - RNA, 19 nt, Artificial
  Human sequences
caaguguccauaccucaau

SEQ ID NO: 140 - RNA, 19 nt, Artificial
  Human sequence
ucagguguuauccacguga

SEQ ID NO: 141 - RNA, 19 nt, Artificial
  Human sequences
uggcugaagugacguuauc

SEQ ID NO: 142 - RNA, 19 nt, Artificial
  Human sequences
aggaaugagagauugagaa

SEQ ID NO: 143 - RNA, 19 nt, Artificial
  Human sequences
aagaucugaagguagccuc

SEQ ID NO: 144 - RNA, 19 nt, Artificial
  Human sequences
auguuuccauucugccauc

SEQ ID NO: 145 - RNA, 19 nt, Artificial
  Mouse sequences
aguauuuuggcaggaccag

SEQ ID NO: 146 - RNA, 19 nt, Artificial
  Mouse sequences
cauaaauuugaccugcacg

SEQ ID NO: 147 - RNA, 19 nt, Artificial
  Mouse sequence
caagauaaugucacagaac

SEQ ID NO: 148 - RNA, 19 nt, Artificial
  Human sequence
aguauuuuggcaggaccag

SEQ ID NO: 149 - RNA, 19 nt, Artificial
  Human sequence
cauaaauuugaccugcuca

SEQ ID NO: 150 - RNA, 19 nt, Artificial
  Human sequence
caagauaaugucacagaac

SEQ ID NO: 151 - RNA, 19 nt, Artificial
  Mouse sequence
agcacagauaccaccaaga

SEQ ID NO: 152 - RNA, 19 nt, Artificial
  Mouse
accaucaagaucaauggcu

SEQ ID NO: 153 - RNA, 19 nt, Artificial
  Mouse sequence
aacacugccgagcucaaga

SEQ ID NO: 154 - RNA, 19 nt, Artificial
  Mouse sequence
agcucaagaucugccgagu

SEQ ID NO: 155 - RNA, 19 nt, Artificial
  Mouse sequence
auugcggacauggacuucu

SEQ ID NO: 156 - RNA, 19 nt, Artificial
  Mouse sequences
ugagucagaucagcuccua

SEQ ID NO: 157 - RNA, 19 nt, Artificial
  Human sequences
agcacagauaccaccaaga

SEQ ID NO: 158 - RNA, 19 nt, Artificial
  Human sequences
accaucaagaucaauggcu

SEQ ID NO: 159 - RNA, 19 nt, Artificial
  Human sequence
aacacugccgagcucaaga

SEQ ID NO: 160 - RNA, 19 nt, Artificial
  Human sequences
agcucaagaucugccgagu

SEQ ID NO: 161 - RNA, 19 nt, Artificial
  Human sequence
auugcggacauggacuucu

SEQ ID NO: 162 - RNA, 19 nt, Artificial
  Human sequence
ugagucagaucagcuccua

SEQ ID NO: 163 - RNA, 19 nt, Artificial
  Mouse sequence
uuuaacaaccugggcaucc

SEQ ID NO: 164 - RNA, 19 nt, Artificial
  Mouse sequence
cugccauugagcggaagau

SEQ ID NO: 165 - RNA, 19 nt, Artificial
  Mouse sequence
cuccuggacgauggcuuug

SEQ ID NO: 166 - RNA, 19 nt, Artificial
  Mouse sequence
uugugggcagcaacauguu

SEQ ID NO: 167 - RNA, 19 nt, Artificial
  Human sequence
uuuaacaaccugggcaucc

SEQ ID NO: 168 - RNA, 19 nt, Artificial
  Human sequence
cugccauugagcggaagau

SEQ ID NO: 169 - RNA, 19 nt, Artificial
  Human sequence
cuccuggacgauggcuuug

SEQ ID NO: 170 - RNA, 19 nt, Artificial
  Human sequence
uugugggcagcaacauguu

SEQ ID NO: 171 - RNA, 19 nt, Artificial
  Mouse sequence
uagucgcuuugguuuugcu

SEQ ID NO: 172 - RNA, 19 nt, Artificial
  Mouse sequence
agagaauaaugcagaccag

SEQ ID NO: 173 - RNA, 19 nt, Artificial
  Mouse sequence
uauccuucuugugacuccu

SEQ ID NO: 174 - RNA, 19 nt, Artificial
  Mouse sequence
uccucaagcugcuauguuu

SEQ ID NO: 175 - RNA, 19 nt, Artificial
  Human sequence
aauguucugcugaucgaug

SEQ ID NO: 176 - RNA, 19 nt, Artificial
  Human sequence
ugagcuacaaagaggacac

SEQ ID NO: 177 - RNA, 19 nt, Artificial
  Human sequence
aggauccugaguuugugaa

SEQ ID NO: 178 - RNA, 19 nt, Artificial
  Human sequence
cuguaaugugccagcauug

SEQ ID NO: 179 - RNA, 19 nt, Artificial
  Human sequence
cuguaaugugccagcauug

SEQ ID NO: 180 - RNA, 19 nt, Artificial
  Human sequence
uaugguaauacgugaggaa

SEQ ID NO: 181 - RNA, 19 nt, Artificial
  Mouse sequence
uaugguaauacgugaggaa

SEQ ID NO: 182 - RNA, 19 nt, Artificial
  Mouse sequence
ggauuucuuccagagcugu

SEQ ID NO: 183 - RNA, 19 nt, Artificial
  Mouse sequence
guuacaaguccaucuuugu

SEQ ID NO: 184 - RNA, 19 nt, Artificial
  Human sequence
cuuggauuugucccacaac

SEQ ID NO: 185 - RNA, 19 nt, Artificial
  Human sequence
ugauuucuuccagagcugc

SEQ ID NO: 186 - RNA, 19 nt, Artificial
  Human sequence
guuacaaguccaucuuugu

SEQ ID NO: 187 - RNA, 19 nt, Artificial
  Mouse sequence
gaaaagccuugaccugucu

SEQ ID NO: 188 - RNA, 19 nt, Artificial
  Mouse sequence
cgaacuggacuucucccac

SEQ ID NO: 189 - RNA, 19 nt, Artificial
  Human sequence
aaaaagccuugaccugucc

SEQ ID NO: 190 - RNA, 19 nt, Artificial
  Mouse sequence
ugaacuggacuucucccau

SEQ ID NO: 191 - RNA, 19 nt, Artificial
  Mouse sequence
gaaguggacaaaucucacc

SEQ ID NO: 192 - RNA, 19 nt, Artificial
  Mouse sequence
gccaggaauggagaggucu

SEQ ID NO: 193 - RNA, 19 nt, Artificial
  Mouse sequence
cucuucguaacuugaccau

SEQ ID NO: 194 - RNA, 19 nt, Artificial
  Mouse sequence
aagcaacaacaacauagcc

SEQ ID NO: 195 - RNA, 19 nt, Artificial
  Mouse sequence
cugucucaccuccacaucu

SEQ ID NO: 196 - RNA, 19 nt, Artificial
  Mouse sequence
cugcacgugugaaaguauu

SEQ ID NO: 197 - RNA, 19 nt, Artificial
  Mouse sequence
gaagauucaagguacauca

SEQ ID NO: 198 - RNA, 19 nt, Artificial
  Human sequence
aaaguggacaaaucucacu

SEQ ID NO: 199 - RNA, 19 nt, Artificial
  Human sequence
gccaggaauggagaggucu

SEQ ID NO: 200 - RNA, 19 nt, Artificial
  Human sequence
cucuucguaacuugaccau

SEQ ID NO: 201 - RNA, 19 nt, Artificial
  Human sequence
aagcaacaacaacauagcc

SEQ ID NO: 202 - RNA, 19 nt, Artificial
  Human sequence
cugucucaccuccacaucc

SEQ ID NO: 203 - RNA, 19 nt, Artificial
  Human sequence
uugcacgugugaaaguauu

SEQ ID NO: 204 - RNA, 19 nt, Artificial
  Human sequence
aaagauucaagguacauca

SEQ ID NO: 205 - RNA, 19 nt, Artificial
  Human sequence
aaccaugcacucuguuugc

SEQ ID NO: 206 - RNA, 19 nt, Artificial
  Mouse sequence
uggauuuauccagguguga

SEQ ID NO: 207 - RNA, 19 nt, Artificial
  Mouse sequence
ugguggcuguggagacaaa

SEQ ID NO: 208 - RNA, 19 nt, Artificial
  Mouse sequence
acuacagagacuuuauucc

SEQ ID NO: 209 - RNA, 19 nt, Artificial
  Mouse sequence
uugcugccaacaucaucca

SEQ ID NO: 210 - RNA, 19 nt, Artificial
  Human sequence
uggauuuauccagguguga

SEQ ID NO: 211 - RNA, 19 nt, Artificial
  Human sequence
ugguggcuguggagacaaa

SEQ ID NO: 212 - RNA, 19 nt, Artificial
  Human sequence
acuacagagacuuuauucc

SEQ ID NO: 213 - RNA, 19 nt, Artificial
  Human sequence
acuacagagacuuuauucc

SEQ ID NO: 214 - RNA, 19 nt, Artificial
  Mouse sequence
cagcuucaacuauaucagu

SEQ ID NO: 215 - RNA, 19 nt, Artificial
  Mouse sequence
cuuugcucaaacaccugga

SEQ ID NO: 216 - RNA, 19 nt, Artificial
  Human sequence
gagcuucaacuauaucagg

SEQ ID NO: 217 - RNA, 19 nt, Artificial
  Human sequence
cuuugcucaaacaccugga

SEQ ID NO: 218 - RNA, 19 nt, Artificial
  Mouse sequence
uugcucacuugcaucuaag

SEQ ID NO: 219 - RNA, 19 nt, Artificial
  Mouse sequence
augauucugccugggugaa

SEQ ID NO: 220 - RNA, 19 nt, Artificial
  Human sequence
caugagaggaacuuugucc

SEQ ID NO: 221 - RNA, 19 nt, Artificial
  Human sequence
uugcucacuugcaucuaag

SEQ ID NO: 222 - RNA, 19 nt, Artificial
  Human sequence
augauucugccugggugaa

SEQ ID NO: 223 - RNA, 19 nt, Artificial
  Human sequence
caugagaggaacuuugucc

SEQ ID NO: 224 - RNA, 19 nt, Artificial
  Mouse sequence
gaugguuuccuaaaacucu

SEQ ID NO: 225 - RNA, 19 nt, Artificial
  Mouse sequence
uuuaccuggauggaaacca

SEQ ID NO: 226 - RNA, 19 nt, Artificial
  Mouse sequence
uguuauuaucgaaauccuu

SEQ ID NO: 227 - RNA, 19 nt, Artificial
  Mouse sequence
aagauaacaaugucacagc

SEQ ID NO: 228 - RNA, 19 nt, Artificial
  Mouse sequence
uucuugaccuaaguggaaa

SEQ ID NO: 229 - RNA, 19 nt, Artificial
  Mouse sequence
ccaaacucuuaauggcagu

SEQ ID NO: 230 - RNA, 19 nt, Artificial
  Mouse sequence
cugugaugcugugugguuu

SEQ ID NO: 231 - RNA, 19 nt, Artificial
  Mouse sequence
aaaccugauucuguucuca

SEQ ID NO: 232 - RNA, 19 nt, Artificial
  Human sequence
gaugguuuccuaaaacucu

SEQ ID NO: 233 - RNA, 19 nt, Artificial
  Human sequence
uuuaccuggauggaaacca

SEQ ID NO: 234 - RNA, 19 nt, Artificial
  Human sequence
uuuaccuggauggaaacca

SEQ ID NO: 235 - RNA, 19 nt, Artificial
  Human sequence
aagauaacaaugucacagc

SEQ ID NO: 236 - RNA, 19 nt, Artificial
  Human sequence
uucuugaccuaaguggaaa

SEQ ID NO: 237 - RNA, 19 nt, Artificial
  Human sequence
ccaaacucuuaauggcagu

SEQ ID NO: 238 - RNA, 19 nt, Artificial
  Human sequence
cugugaugcugugugguuu

SEQ ID NO: 239 - RNA, 19 nt, Artificial
  Human sequence
aaaccugauucuguucuca

SEQ ID NO: 240 - RNA, 19 nt, Artificial
  Mouse sequence
acugggauguuugguuuau

SEQ ID NO: 241 - RNA, 19 nt, Artificial
  Mouse sequence
uaaccucaugcagagcaua

SEQ ID NO: 242 - RNA, 19 nt, Artificial
  Mouse sequence
uaaccucaugcagagcaua

SEQ ID NO: 243 - RNA, 19 nt, Artificial
  Mouse sequence
gagaacauggaugugauua

SEQ ID NO: 244 - RNA, 19 nt, Artificial
  Human sequence
acugggauguuugguuuau

SEQ ID NO: 245 - RNA, 19 nt, Artificial
  Human sequence
caaccucaugcagagcauc

SEQ ID NO: 246 - RNA, 19 nt, Artificial
  Human sequence
uugcagaggcuaauggaug

SEQ ID NO: 247 - RNA, 19 nt, Artificial
  Human sequence
gagaacauggaugugauua

SEQ ID NO: 248 - RNA, 19 nt, Artificial
  Mouse sequence
aaccuguccuucaauuacc

SEQ ID NO: 249 - RNA, 19 nt, Artificial
  Mouse sequence
acggcaucuucuuccgcuc

SEQ ID NO: 250 - RNA, 19 nt, Artificial
  Mouse sequence
augaacuucaucaaccagg

SEQ ID NO: 251 - RNA, 19 nt, Artificial
  Mouse sequence
aaggcccugaccaauggca

SEQ ID NO: 252 - RNA, 19 nt, Artificial
  Human sequence
aaccuguccuucaauuacc

SEQ ID NO: 253 - RNA, 19 nt, Artificial
  Human sequence
acggcaucuucuuccgcuc

SEQ ID NO: 254 - RNA, 19 nt, Artificial
  Human sequence
augaacuucaucaaccagg

SEQ ID NO: 255 - RNA, 19 nt, Artificial
  Human sequence
aaggcccugaccaauggca

SEQ ID NO: 256 - RNA, 19 nt, Artificial
  Mouse sequence
gacacucaucauuuucacc

SEQ ID NO: 257 - RNA, 19 nt, Artificial
  Mouse sequence′
gcuaucuggucaaccucgu

SEQ ID NO: 258 - RNA, 19 nt, Artificial
  Mouse sequence
aagcuauggugagaugcag

SEQ ID NO: 259 - RNA, 19 nt, Artificial
  Mouse sequence
cugaggacagcuguccucu

SEQ ID NO: 260 - RNA, 19 nt, Artificial
  Mouse sequence
cagugggaaauauuuagca

SEQ ID NO: 261 - RNA, 19 nt, Artificial
  Mouse sequence
cauguacuugucaaagaag

SEQ ID NO: 262 - RNA, 19 nt, Artificial
  Human sequence
aacacucaucauuuucacu

SEQ ID NO: 263 - RNA, 19 nt, Artificial
  Human sequence
gcuaucuggucaaccuccu

SEQ ID NO: 264 - RNA, 19 nt, Artificial
  Human sequence
aagcuauggugagaugcag

SEQ ID NO: 265 - RNA, 19 nt, Artificial
  Human sequence
cugaggacagcuguccucu

SEQ ID NO: 266 - RNA, 19 nt, Artificial
  Human sequence
cagugggaaauauuuagca

SEQ ID NO: 267 - RNA, 19 nt, Artificial
  Human sequence
uauguacuugucaaagaag

SEQ ID NO: 268 - RNA, 19 nt, Artificial
  Mouse sequence
uuucucucccuggcuuuug

SEQ ID NO: 269 - RNA, 19 nt, Artificial
  Mouse sequence
gagcuacggaacaggugga

SEQ ID NO: 270 - RNA, 19 nt, Artificial
  Human sequence
uuucucucccuggcuuuug

SEQ ID NO: 271 - RNA, 19 nt, Artificial
  Human sequence
aagcuacggaacagguggg

SEQ ID NO: 272 - RNA, 19 nt, Artificial
  Mouse sequence
gaguacaugaauugcacuu

SEQ ID NO: 273 - RNA, 19 nt, Artificial
  Mouse sequence
ugagugaaucccagcuaga

SEQ ID NO: 274 - RNA, 19 nt, Artificial
  Mouse sequence
ccuggaguggugugucuaa

SEQ ID NO: 275 - RNA, 19 nt, Artificial
  Mouse sequence
agccagacuacagugaacg

SEQ ID NO: 276 - RNA, 19 nt, Artificial
  Human sequence
gaguacaugaauugcacuu

SEQ ID NO: 277 - RNA, 19 nt, Artificial
  Human sequence
ugagugaaucccagcuaga

SEQ ID NO: 278 - RNA, 19 nt, Artificial
  Human sequence
ccuggaguggugugucuaa

SEQ ID NO: 279 - RNA, 19 nt, Artificial
  Human sequence
agccagacuacagugaacg

SEQ ID NO: 280 - RNA, 19 nt, Artificial
  Mouse sequence
cagcaaauccuugcuguuc

SEQ ID NO: 281 - RNA, 19 nt, Artificial
  Mouse sequence′
gauuugaccaguauaagug

SEQ ID NO: 282 - RNA, 19 nt, Artificial
  Mouse sequence
caaacacagugcacuacau

SEQ ID NO: 283 - RNA, 19 nt, Artificial
  Mouse sequence
auuugauugacaguccacc

SEQ ID NO: 284 - RNA, 19 nt, Artificial
  Mouse sequence
uacaaaagcugggaagccu

SEQ ID NO: 285 - RNA, 19 nt, Artificial
  Mouse sequence
uucuuugcccagcacuuca

SEQ ID NO: 286 - RNA, 19 nt, Artificial
  Mouse sequence
aagacagaucauaagcgag

SEQ ID NO: 287 - RNA, 19 nt, Artificial
  Mouse sequence
uaaacugcgccuuuucaag

SEQ ID NO: 288 - RNA, 19 nt, Artificial
  Mouse sequence
ccuuuucaaggauggaaaa

SEQ ID NO: 289 - RNA, 19 nt, Artificial
  Mouse sequence
agagaugaucuacccuccu

SEQ ID NO: 290 - RNA, 19 nt, Artificial
  Mouse sequence
gucuuuggucuggugccug

SEQ ID NO: 291 - RNA, 19 nt, Artificial
  Mouse sequence
gucugaugauguaugccac

SEQ ID NO: 292 - RNA, 19 nt, Artificial
  Mouse sequence
cuccauugaccagagcaga

SEQ ID NO: 293 - RNA, 19 nt, Artificial
  Mouse sequence
cuccauugaccagagcaga

SEQ ID NO: 294 - RNA, 19 nt, Artificial
  Mouse sequence
aaugaguaccgcaaacgcu

SEQ ID NO: 295 - RNA, 19 nt, Artificial
  Mouse sequence
uugaagaacuuacaggaga

SEQ ID NO: 296 - RNA, 19 nt, Artificial
  Mouse sequence
uuggagcaccauucuccuu

SEQ ID NO: 297 - RNA, 19 nt, Artificial
  Mouse sequence
acugccucaauucagucuc

SEQ ID NO: 298 - RNA, 19 nt, Artificial
  Human sequence
cagcaaauccuugcuguuc

SEQ ID NO: 299 - RNA, 19 nt, Artificial
  Human sequence
gauuugaccaguauaagug

SEQ ID NO: 300 - RNA, 19 nt, Artificial
  Human sequence
caaacacagugcacuacau

SEQ ID NO: 301 - RNA, 19 nt, Artificial
  Human sequence
auuugauugacaguccacc

SEQ ID NO: 302 - RNA, 19 nt, Artificial
  Human sequence
uacaaaagcugggaagccu

SEQ ID NO: 303 - RNA, 19 nt, Artificial
  Human sequence
uucuuugcccagcacuuca

SEQ ID NO: 304 - RNA, 19 nt, Artificial
  Human sequence
aagacagaucauaagcgag

SEQ ID NO: 305 - RNA, 19 nt, Artificial
  Human sequence
uaaacugcgccuuuucaag

SEQ ID NO: 306 - RNA, 19 nt, Artificial
  Human sequence
ccuuuucaaggauggaaaa

SEQ ID NO: 307 - RNA, 19 nt, Artificial
  Human sequence
agagaugaucuacccuccu

SEQ ID NO: 308 - RNA, 19 nt, Artificial
  Human sequence
gucuuuggucuggugccug

SEQ ID NO: 309 - RNA, 19 nt, Artificial
  Human sequence
gucugaugauguaugccac

SEQ ID NO: 310 - RNA, 19 nt, Artificial
  Human sequence
uuccauugaccagagcagg

SEQ ID NO: 311 - RNA, 19 nt, Artificial
  Human sequence
cagaugaaauaccagucuu

SEQ ID NO: 312 - RNA, 19 nt, Artificial
  Human sequence
aaugaguaccgcaaacgcu

SEQ ID NO: 313 - RNA, 19 nt, Artificial
  Human sequence
uugaagaacuuacaggaga

SEQ ID NO: 314 - RNA, 19 nt, Artificial
  Human sequence
uuggagcaccauucuccuu

SEQ ID NO: 315 - RNA, 19 nt, Artificial
  Human sequence
acugccucaauucagucuc

SEQ ID NO: 316 - DNA, 19 bp, Artificial
  Antisense sequence
ttggtcattgtgagtgcca

SEQ ID NO: 317 - RNA, 19 nt, Artificial
  Antisense sequence
uggcacucacaaugaccaa

SEQ ID NO: 318 - DNA, 23 bp, Artificial
  Sense strand
uuggucauugugagugccadtdt

SEQ ID NO: 319 - DNA, 23 bp, Artificial
  Antisense sequence
uggcacucacaaugaccaadtdt

SEQ ID NO: 320 - RNA, 21 nt, Artificial
  Antisense sequence
ggcuacguccaggagcgcauu

SEQ ID NO: 321 - RNA, 21 nt, Artificial
  Antisense sequence
ugcgcuccuggacguagccuu

SEQ ID NO: 322 - DNA, 46 bp, Artificial
  Shows schematic diagrams of HFceRa-338 and GFP-949 siRNA and their hairpin derivatives/precursors.
gaauauugugaaugccaaadtdtdtdtcuuguaacacuuacgguuu

SEQ ID NO: 323 - RNA, 60 nt, Artificial
  Shows schematic diagrams of HFceRa-338 and GFP-949 siRNA and their hairpin derivatives/precursors.
cggggugaauauugugaaugccaaagcagguccauuuggcauucacaauguucuucccug

SEQ ID NO: 324 - RNA, 60 nt, Artificial
  Shows schematic diagrams of HFceRa-338 and GFP-949 siRNA and their hairpin derivatives/precursors.
cgggguugcgcuccuggacguagccgcagguccaggcuacguccaggagcgcauucccug

SEQ ID NO: 325 - RNA, 119 nt, Artificial
  Shows schematic diagrams of HFceRa-338 and GFP-949 siRNA and their hairpin derivatives/precursors.
cggggugaauauugugaaugccaaagcagcgggguugcgcuccuggacguagccgcagguccaggcuacguccaggagcgcauucccuguccauuuggcauucacaauguucuucccug

SEQ ID NO: 326 - RNA, 119 nt, Artificial
  Shows schematic diagrams of HFceRa-338 and GFP-949 siRNA and their hairpin derivatives/precursors.
cgggguugcgcuccuggacguagccgcagcggggugaauauugugaaugccaaagcagguccauuuggcauucacaauguucuucccuguccaggcuacguccaggagcgcauucccug